SEQUENCE LISTING (41 sequences)

SEQ ID NO: 1 - DNA, 918 bp, Artificial Sequence
  Human Notch1 polynucleotide encoding amino acids 1427-1732
cacatcctggactacagcttcgggggtggggccgggcgcgacatccccccgccgctgatcgaggaggcgtgcgagctgcccgagtgccaggaggacgcgggcaacaaggtctgcagcctgcagtgcaacaaccacgcgtgcggctgggacggcggtgactgctccctcaacttcaatgacccctggaagaactgcacgcagtctctgcagtgctggaagtacttcagtgacggccactgtgacagccagtgcaactcagccggctgcctcttcgacggctttgactgccagcgtgcggaaggccagtgcaaccccctgtacgaccagtactgcaaggaccacttcagcgacgggcactgcgaccagggctgcaacagcgcggagtgcgagtgggacgggctggactgtgcggagcatgtacccgagaggctggcggccggcacgctggtggtggtggtgctgatgccgccggagcagctgcgcaacagctccttccacttcctgcgggagctcagccgcgtgctgcacaccaacgtggtcttcaagcgtgacgcacacggccagcagatgatcttcccctactacggccgcgaggaggagctgcgcaagcaccccatcaagcgtgccgccgagggctgggccgcacctgacgccctgctgggccaggtgaaggcctcgctgctccctggtggcagcgagggtgggcggcggcggagggagctggaccccatggacgtccgcggctccatcgtctacctggagattgacaaccggcagtgtgtgcaggcctcctcgcagtgcttccagagtgccaccgacgtggccgcattcctgggagcgctcgcctcgctgggcagcctcaacatcccctacaagatcgaggccgtgcagagtgagaccgtggagccgcccccgccg

SEQ ID NO: 2 - protein, 306 aa, Artificial Sequence
  Human Notch1 amino acids 1427-1732
HILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPP

SEQ ID NO: 3 - DNA, 1395 bp, Artificial Sequence
  52M51 Heavy chain polynucleotide sequence
atggaatggacctgggtctttctcttcctcctgtcagtaactgcaggtgtccactcccaggttcagctgcagcagtctggagctgagctgatgaagcctggggcctcagtgaagatatcctgcaaggctgctggctacacaatgagaggctactggatagagtggataaagcagaggcctggacatggccttgagtggattggacagattttacctggaactgggagaactaactacaatgagaagttcaagggcaaggccacattcactgcagatacatcctccaacacagccaacatgcaactcagcagcctgacatctgaggactctgccgtctattactgtgcaagatttgatggtaactacggttactatgctatggactactggggtcaaggatcctcagtcaccgtctcctcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagccctcggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtcctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatatccaaaaccaaaggcagaccgaaggctccacaggtgtacaccattccacctcccaaggagcagatggccaaggataaagtcagtctgacctgcatgataacagacttcttccctgaagacataacagtggagtggcagtggaatgggcagccagcggagaactacaagaacactcagcccatcatgaacacgaatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 4 - protein, 464 aa, Artificial Sequence
  52M51 Heavy chain amino acid sequence
MEWTWVFLFLLSVTAGVHSQVQLQQSGAELMKPGASVKISCKAAGYTMRGYWIEWIKQRPGHGLEWIGQILPGTGRTNYNEKFKGKATFTADTSSNTANMQLSSLTSEDSAVYYCARFDGNYGYYAMDYWGQGSSVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 5 - DNA, 420 bp, Artificial Sequence
  52M51 Heavy chain variable region polynucleotide sequence
atggaatggacctgggtctttctcttcctcctgtcagtaactgcaggtgtccactcccaggttcagctgcagcagtctggagctgagctgatgaagcctggggcctcagtgaagatatcctgcaaggctgctggctacacaatgagaggctactggatagagtggataaagcagaggcctggacatggccttgagtggattggacagattttacctggaactgggagaactaactacaatgagaagttcaagggcaaggccacattcactgcagatacatcctccaacacagccaacatgcaactcagcagcctgacatctgaggactctgccgtctattactgtgcaagatttgatggtaactacggttactatgctatggactactggggtcaaggatcctcagtcaccgtctcctca

SEQ ID NO: 6 - protein, 180 aa, Artificial Sequence
  52M51 Heavy chain variable region amino acid sequence
MEWTWVFLFLLSVTAGVHSQVQLQQSGAELMKPGASVKISCKAAGYTMRGYWIEWIKQRPGHGLEWIGQILPGTGRTNYNEKFKGKATFTADTSSNTANMQLSSLTSEDSAVYYCARFDGNYGYYAMDYWGQGSSVTVSSAKTTPPSVYPLAPGSAAQTNSMVTLGCLVKGYFPEPVTVT

SEQ ID NO: 7 - DNA, 363 bp, Artificial Sequence
  52M51 Heavy chain variable region polynucleotide sequence without predicted signal sequence
caggttcagctgcagcagtctggagctgagctgatgaagcctggggcctcagtgaagatatcctgcaaggctgctggctacacaatgagaggctactggatagagtggataaagcagaggcctggacatggccttgagtggattggacagattttacctggaactgggagaactaactacaatgagaagttcaagggcaaggccacattcactgcagatacatcctccaacacagccaacatgcaactcagcagcctgacatctgaggactctgccgtctattactgtgcaagatttgatggtaactacggttactatgctatggactactggggtcaaggatcctcagtcaccgtctcctca

SEQ ID NO: 8 - protein, 122 aa, Artificial Sequence
  52M51 Heavy chain variable region amino acid sequence without predicted signal sequence
QVQLQQSGAELMKPGASVKISCKAAGYTMRGYWIEWIKQRPGHGLEWIGQILPGTGRTNYNEKFKGKATFTADTSSNTANMQLSSLTSEDSAVYYCARFDGNYGYYAMDYWGQGSSVTVSSA

SEQ ID NO: 9 - DNA, 705 bp, Artificial Sequence
  52M51 Light chain polynucleotide sequence
atggcctggatttcacttatactctctctcctggctctcagctcaggggccatttcccaggctgttgtgactcaggaatctgcactcaccacatcacctggtgaaacagtcacactcacttgtcgctcaagtactggggctgttacaactagtaactacgccaactgggtccaagaaaaacctgatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccactgggtgttcggtggaggaaccaaactgactgtcctaggccagcccaagtcttcgccatcagtcaccctgtttccaccttcctctgaagagctcgagactaacaaggccacactggtgtgtacgatcactgatttctacccaggtgtggtgacagtggactggaaggtagatggtacccctgtcactcagggtatggagacaacccagccttccaaacagagcaacaacaagtacatggctagcagctacctgaccctgacagcaagagcatgggaaaggcatagcagttacagctgccaggtcactcatgaaggtcacactgtggagaagagtttgtcccgtgctgactgttcctag

SEQ ID NO: 10 - protein, 234 aa, Artificial Sequence
  52M51 Light chain amino acid sequence
MAWISLILSLLALSSGAISQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 11 - DNA, 387 bp, Artificial Sequence
  52M51 Light chain variable region polynucleotide sequence
atggcctggatttcacttatactctctctcctggctctcagctcaggggccatttcccaggctgttgtgactcaggaatctgcactcaccacatcacctggtgaaacagtcacactcacttgtcgctcaagtactggggctgttacaactagtaactacgccaactgggtccaagaaaaacctgatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccactgggtgttcggtggaggaaccaaactgactgtcctaggc

SEQ ID NO: 12 - protein, 180 aa, Artificial Sequence
  52M51 Light chain variable region amino acid sequence
MAWISLILSLLALSSGAISQAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGQPKSSPSVTLFPPSSEELETNKATLVCTITDFYPGVVTVDWKVDGTPVTQG

SEQ ID NO: 13 - DNA, 330 bp, Artificial Sequence
  52M51 Light chain variable region polynucleotide sequence without predicted signal sequence
caggctgttgtgactcaggaatctgcactcaccacatcacctggtgaaacagtcacactcacttgtcgctcaagtactggggctgttacaactagtaactacgccaactgggtccaagaaaaacctgatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccactgggtgttcggtggaggaaccaaactgactgtcctaggc

SEQ ID NO: 14 - protein, 110 aa, Artificial Sequence
  52M51 Light chain variable region amino acid sequence without predicted signal sequence
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLG

SEQ ID NO: 15 - protein, 6 aa, Artificial Sequence
  52M51 Heavy chain CDR1
RGYWIE

SEQ ID NO: 16 - protein, 17 aa, Artificial Sequence
  52M51 Heavy chain CDR2
QILPGTGRTNYNEKFKG

SEQ ID NO: 17 - protein, 12 aa, Artificial Sequence
  52M51 Heavy chain CDR3
FDGNYGYYAMDY

SEQ ID NO: 18 - protein, 14 aa, Artificial Sequence
  52M51 Light chain CDR1
RSSTGAVTTSNYAN

SEQ ID NO: 19 - protein, 7 aa, Artificial Sequence
  52M51 Light chain CDR2
GTNNRAP

SEQ ID NO: 20 - protein, 16 aa, Artificial Sequence
  52M51 Light chain CDR3
ALWYSNHWVFGGGTKL

SEQ ID NO: 21 - DNA, 1401 bp, Artificial Sequence
  52M51-H4 Heavy chain polynucleotide sequence
atggattggacatggagggtgttctgcctcctcgctgtggctcctggagtcctgagccaggtccagctcgtccagagcggggctgaagtcaagaagcctggcgctagcgtcaaaatcagctgtaaggtcagcggatacacactgaggggatactggatcgagtgggtgaggcaggctccaggaaagggcctggaatggatcggccagatcctgcctggaaccggaaggacaaattacaatgagaagtttaagggaagggtcacaatgacagcagacacaagcacagacacagcttatatggaactcagctccctcagatccgaggacaccgctgtctactattgtgccaggttcgatggaaattacggatactatgccatggattactggggacaggggacaacggtcaccgtgagctcagccagcacaaagggccctagcgtcttccctctggctccctgcagcaggagcaccagcgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 22 - protein, 466 aa, Artificial Sequence
  52M51-H4 Heavy chain amino acid sequence
MDWTWRVFCLLAVAPGVLSQVQLVQSGAEVKKPGASVKISCKVSGYTLRGYWIEWVRQAPGKGLEWIGQILPGTGRTNYNEKFKGRVTMTADTSTDTAYMELSSLRSEDTAVYYCARFDGNYGYYAMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - protein, 447 aa, Artificial Sequence
  52M51-H4 Heavy chain amino acid sequence without predicted signal sequence
QVQLVQSGAEVKKPGASVKISCKVSGYTLRGYWIEWVRQAPGKGLEWIGQILPGTGRTNYNEKFKGRVTMTADTSTDTAYMELSSLRSEDTAVYYCARFDGNYGYYAMDYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 24 - DNA, 423 bp, Artificial Sequence
  52M51-H4  Heavy chain variable region polynucleotide sequence
atggattggacatggagggtgttctgcctcctcgctgtggctcctggagtcctgagccaggtccagctcgtccagagcggggctgaagtcaagaagcctggcgctagcgtcaaaatcagctgtaaggtcagcggatacacactgaggggatactggatcgagtgggtgaggcaggctccaggaaagggcctggaatggatcggccagatcctgcctggaaccggaaggacaaattacaatgagaagtttaagggaagggtcacaatgacagcagacacaagcacagacacagcttatatggaactcagctccctcagatccgaggacaccgctgtctactattgtgccaggttcgatggaaattacggatactatgccatggattactggggacaggggacaacggtcaccgtgagctcagcc

SEQ ID NO: 25 - protein, 141 aa, Artificial Sequence
  52M51-H4  Heavy chain variable region amino acid sequence
MDWTWRVFCLLAVAPGVLSQVQLVQSGAEVKKPGASVKISCKVSGYTLRGYWIEWVRQAPGKGLEWIGQILPGTGRTNYNEKFKGRVTMTADTSTDTAYMELSSLRSEDTAVYYCARFDGNYGYYAMDYWGQGTTVTVSSA

SEQ ID NO: 26 - protein, 122 aa, Artificial Sequence
  52M51-H4  Heavy chain variable region amino acid sequence without predicted signal sequence
QVQLVQSGAEVKKPGASVKISCKVSGYTLRGYWIEWVRQAPGKGLEWIGQILPGTGRTNYNEKFKGRVTMTADTSTDTAYMELSSLRSEDTAVYYCARFDGNYGYYAMDYWGQGTTVTVSSA

SEQ ID NO: 27 - DNA, 720 bp, Artificial Sequence
  52M51-L3 Light chain polynucleotide sequence
atgagcgtccctacaatggcttggatgatgctcctgctgggactcctggcttatggaagcggagtggatagccaggccgtcgtcacacaggaacctagcctcaccgttagccctggaggaacagtcacactgacctgtaggagctccacaggagctgtgacaacaagcaattacgctaactggttccagcagaagcccggtcaagcccctagaaccctcatcggcggcaccaataacagagctcccggagtccccgccaggttctccggctccctcctgggtggcaaggctgctctgacactcagcggtgcccagccagaggatgaagcggagtactactgtgcactgtggtacagcaaccattgggttttcggaggcggaacaaagttaaccgtcctcgggcagcctaaggctgctcctagcgtcacactgttccccccatctagcgaggagctgcaggctaacaaggcaaccctcgtctgcctggttagcgacttctaccctggcgctgtcacagtggcctggaaagctgacggctcccctgtgaaagttggcgtcgaaaccacaaagccttctaagcagagcaataataaatatgccgcaagctcctacctctccctgactcctgagcagtggaaaagccataggagctactcctgccgggtcacacacgaaggaagcacagtggaaaagacagtcgcccctgctgagtgtagctga

SEQ ID NO: 28 - protein, 239 aa, Artificial Sequence
  52M51-L3 Light chain amino acid sequence
MSVPTMAWMMLLLGLLAYGSGVDSQAVVTQEPSLTVSPGGTVTLTCRSSTGAVTTSNYANWFQQKPGQAPRTLIGGTNNRAPGVPARFSGSLLGGKAALTLSGAQPEDEAEYYCALWYSNHWVFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 29 - protein, 220 aa, Artificial Sequence
  52M51-L3 Light chain amino acid sequence without predicted signal sequence
SGVDSQAVVTQEPSLTVSPGGTVTLTCRSSTGAVTTSNYANWFQQKPGQAPRTLIGGTNNRAPGVPARFSGSLLGGKAALTLSGAQPEDEAEYYCALWYSNHWVFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 30 - DNA, 402 bp, Artificial Sequence
  52M51-L3 Light chain variable region polynucleotide sequence
atgagcgtccctacaatggcttggatgatgctcctgctgggactcctggcttatggaagcggagtggatagccaggccgtcgtcacacaggaacctagcctcaccgttagccctggaggaacagtcacactgacctgtaggagctccacaggagctgtgacaacaagcaattacgctaactggttccagcagaagcccggtcaagcccctagaaccctcatcggcggcaccaataacagagctcccggagtccccgccaggttctccggctccctcctgggtggcaaggctgctctgacactcagcggtgcccagccagaggatgaagcggagtactactgtgcactgtggtacagcaaccattgggttttcggaggcggaacaaagttaaccgtcctcggg

SEQ ID NO: 31 - protein, 134 aa, Artificial Sequence
  52M51-L3 Light chain variable region amino acid sequence
MSVPTMAWMMLLLGLLAYGSGVDSQAVVTQEPSLTVSPGGTVTLTCRSSTGAVTTSNYANWFQQKPGQAPRTLIGGTNNRAPGVPARFSGSLLGGKAALTLSGAQPEDEAEYYCALWYSNHWVFGGGTKLTVLG

SEQ ID NO: 32 - protein, 115 aa, Artificial Sequence
  52M51-L3 Light chain variable region amino acid sequence without predicted signal sequence
SGVDSQAVVTQEPSLTVSPGGTVTLTCRSSTGAVTTSNYANWFQQKPGQAPRTLIGGTNNRAPGVPARFSGSLLGGKAALTLSGAQPEDEAEYYCALWYSNHWVFGGGTKLTVLG

SEQ ID NO: 33 - DNA, 720 bp, Artificial Sequence
  52M51-L4 Light chain polynucleotide sequence
atgagcgtccctacaatggcttggatgatgctcctgctgggactcctggcttatggaagcggagtggatagccagaccgtcgtcacacaggaacctagcttttccgttagccctggaggaacagtcacactgacctgtaggagctccacaggagctgtgacaacaagcaattacgctaactggtatcagcagactcccggtcaagcccctagaaccctcatcggcggcaccaataacagagctcccggagtccccgacaggttctccggctccatcctgggaaataaagctgctctgacaatcacaggtgcccaggctgacgatgaaagcgactactactgtgcactgtggtacagcaaccattgggttttcggaggcggaacaaagttaaccgtcctcgggcagcctaaggctgctcctagcgtcacactgttccccccatctagcgaggagctgcaggctaacaaggcaaccctcgtctgcctggttagcgacttctaccctggcgctgtcacagtggcctggaaagctgacggctcccctgtgaaagttggcgtcgaaaccacaaagccttctaagcagagcaataataaatatgccgcaagctcctacctctccctgactcctgagcagtggaaaagccataggagctactcctgccgggtcacacacgaaggaagcacagtggaaaagacagtcgcccctgctgagtgtagctga

SEQ ID NO: 34 - protein, 239 aa, Artificial Sequence
  52M51-L4 Light chain amino acid sequence
MSVPTMAWMMLLLGLLAYGSGVDSQTVVTQEPSFSVSPGGTVTLTCRSSTGAVTTSNYANWYQQTPGQAPRTLIGGTNNRAPGVPDRFSGSILGNKAALTITGAQADDESDYYCALWYSNHWVFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 35 - protein, 220 aa, Artificial Sequence
  52M51-L4 Light chain amino acid sequence without predicted signal sequence
SGVDSQTVVTQEPSFSVSPGGTVTLTCRSSTGAVTTSNYANWYQQTPGQAPRTLIGGTNNRAPGVPDRFSGSILGNKAALTITGAQADDESDYYCALWYSNHWVFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 36 - DNA, 402 bp, Artificial Sequence
  52M51-L4 Light chain variable region polynucleotide sequence
atgagcgtccctacaatggcttggatgatgctcctgctgggactcctggcttatggaagcggagtggatagccagaccgtcgtcacacaggaacctagcttttccgttagccctggaggaacagtcacactgacctgtaggagctccacaggagctgtgacaacaagcaattacgctaactggtatcagcagactcccggtcaagcccctagaaccctcatcggcggcaccaataacagagctcccggagtccccgacaggttctccggctccatcctgggaaataaagctgctctgacaatcacaggtgcccaggctgacgatgaaagcgactactactgtgcactgtggtacagcaaccattgggttttcggaggcggaacaaagttaaccgtcctcggg

SEQ ID NO: 37 - protein, 134 aa, Artificial Sequence
  52M51-L4  Light chain variable region amino acid sequence
MSVPTMAWMMLLLGLLAYGSGVDSQTVVTQEPSFSVSPGGTVTLTCRSSTGAVTTSNYANWYQQTPGQAPRTLIGGTNNRAPGVPDRFSGSILGNKAALTITGAQADDESDYYCALWYSNHWVFGGGTKLTVLG

SEQ ID NO: 38 - protein, 115 aa, Artificial Sequence
  52M51-L4  Light chain variable region amino acid sequence without predicted signal sequence
SGVDSQTVVTQEPSFSVSPGGTVTLTCRSSTGAVTTSNYANWYQQTPGQAPRTLIGGTNNRAPGVPDRFSGSILGNKAALTITGAQADDESDYYCALWYSNHWVFGGGTKLTVLG

SEQ ID NO: 39 - protein, 8 aa, Artificial Sequence
  FLAG-tag
DYKDDDDK

SEQ ID NO: 40 - protein, 393 aa, Artificial Sequence [sequence omitted]

SEQ ID NO: 41 - protein, 2555 aa, Artificial Sequence
  Human Notch1
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

(19) United States
(12) Patent Application Publication
(10) Pub. No.: US 2017/0022289 A1
(43) Pub. Date: Jan. 26, 2017

(54) METHODS FOR TREATING CANCER WITH NOTCH1 ANTIBOIDES

(71) Applicant: ONCOMED PHARMACEUTICALS, INC., Redwood City, CA (US)

(72) Inventors: Jakob Dupont, Hillsborough, CA (US); Ann M. Kapoun, Mountain View, CA (US); Belinda Cancilla, Redwood City, CA (US)

(73) Assignee: ONCOMED PHARMACEUTICALS, INC., Redwood City, CA (US)

(21) Appl. No.: 15/123,864

(22) PCT Filed: Mar. 4, 2015
(86) PCT No.: PCT/US15/18756

§ 371 Date: Sep. 6, 2016

Related U.S. Application Data
(60) Provisional application No. 61/949,511, filed on Mar. 7, 2014.
(60) Provisional application No. 62/013,226, filed on Jun. 17, 2014.

(51) Int. Cl. C07K 16/32; C12Q 1/68; G01N 33/574; A61K 39/395; A61K 45/06

(52) U.S. Cl. CPC C07K 16/32; A61K 39/39558; A61K 45/06; G01N 33/57407; C12Q 1/6886; C07K 2317/76; C07K 2317/565; A61K 2039/505

(57) ABSTRACT

The present invention provides methods for treating cancer. More particularly, the invention provides methods for treating cancer comprising administrating doses of an anti-Notch 1 antibody.

### FIELD OF THE INVENTION

The present invention relates to the field of treating cancer. More particularly, the invention provides methods for treating cancer comprising administering anti-Notch1 antibodies.

### BACKGROUND OF THE INVENTION

Cancer is one of the leading causes of mortality in the developed world, with over one million people diagnosed with cancer and 500,000 deaths per year in the United States alone. Overall it is estimated that more than 1 in 3 people will develop some form of cancer during their lifetime.

Adenocarcinomas are a type of cancer that begins in glandular (secretory) cells. Glandular cells are found in tissue that line certain internal organs and make and release substances in the body, such as mucus, digestive juices, or other fluids. Adenocarcinomas include, but are not limited to, cancer of the breast, pancreas, lung, prostate, and colon. Adenoid cystic carcinoma (ACC or AdCC) is a rare form of adenocarcinoma often occurring in the salivary glands, but also can be found in other locations of the body. ACC is generally characterized by an indolent clinical course with multiple local recurrences and distant metastases. The most common site of metastasis is the lung, but metastasis has been found in bone, liver, kidney, and brain. Treatment usually consists of surgical resection and/or adjuvant post-operative radiotherapy, with chemotherapy usually reserved for treatment of advanced local or metastatic disease.

Increasingly, treatment of cancer has moved from the use of systemically acting cytotoxic drugs to include more targeted therapies that hone in on the mechanisms that allow and support unregulated cell growth and survival. For example, tumor angiogenesis, the process by which a tumor establishes an independent blood supply, is a critical step for tumor growth. Efforts to target tumor angiogenesis have emerged as an important strategy for the development of novel cancer therapeutics, such as the anti-VEGF antibody AVASTIN.

Under normal conditions signaling pathways connect extracellular signals to the nucleus, leading to the expression of genes that directly or indirectly control cell growth, cell differentiation, cell survival, and cell death. In a wide variety of cancers, signaling pathways are dysregulated and may be linked to tumor initiation and/or tumor progression. Signaling pathways implicated in human oncogenesis include, but are not limited to, the Notch pathway, the Ras-Raf-MEK-ERK or MAPK pathway, the PI3K-AKT pathway, the CDKN2A/CDK4 pathway, the Bcl-2/TP53 pathway, and the Wnt pathway.

The Notch signaling pathway is a universally conserved signal transduction system. It is involved in cell fate determination during development including embryonic pattern formation and post-embryonic tissue maintenance. In addition, Notch signaling has been identified as a critical factor in the maintenance of hematopoietic stem cells.

The Notch pathway has been linked to the pathogenesis of both hematologic and solid tumors and cancers. Numerous cellular functions and microenvironmental cues associated with tumorigenesis have been shown to be modulated by Notch pathway signaling, including cell proliferation, apoptosis, adhesion, and angiogenesis (Leong et al., 2006, Blood, 107:2223-2233). In addition, Notch receptors and/or Notch ligands have been shown to play potential oncogenic roles in a number of human cancers (Leong et al., 2006, Blood, 107:2223-2233; Nickoloff et al., 2003, Oncogene, 22:6598-6608). Thus, the Notch pathway has been identified as a potential target for cancer therapy.

As drug discovery and drug development advances, especially in the cancer field, the “one drug fits all” approach is shifting to a “personalized medicine” strategy. Personalized medicine strategies may include treatment regimens that are based upon cancer biomarkers, including prognostic markers, pharmacodynamic markers, and predictive markers. In general, predictive biomarkers assess the likelihood that a tumor or cancer will be responsive to or sensitive to a specific therapeutic agent, and may allow for the identification and/or the selection of patients most likely to benefit from the use of that agent.

Therefore, there is a need for designing new and targeted therapeutic strategies that can overcome the relative ineffectiveness of current therapies for treatment of cancer. Furthermore, there is a clear need to develop assays that are capable of predicting and/or identifying whether a tumor/cancer will respond to a particular agent. This information should allow for better patient selection strategies and lead to better therapeutic efficacy.

### SUMMARY OF THE INVENTION

One aspect of the present invention provides methods of treating an adenocarcinoma in a subject comprising administering to the subject a therapeutically effective amount of a Notch1-binding agent. Methods of treating an adenoid cystic carcinoma in a subject comprising administering to the subject a therapeutically effective amount of a Notch1-binding agent are also provided. In addition, methods of inhibiting growth of an adenocarcinoma or an adenoid cystic carcinoma comprising contacting the carcinoma with an effective amount of a Notch1-binding agent are provided. Methods of decreasing the size of an adenoid cystic carcinoma comprising contacting the carcinoma with an effective amount of a Notch1-binding agent are provided. Methods of decreasing the size of an adenoid cystic carcinoma in a subject comprising administering to the subject a therapeutically effective amount of a Notch1-binding agent are provided.

Another aspect of the invention provides methods of decreasing pain in a subject with an adenocarcinoma comprising administering to the subject a therapeutically effective amount of a Notch1-binding agent. Methods of decreasing pain in a subject with an adenoid cystic carcinoma comprising administering to the subject a therapeutically effective amount of a Notch1-binding agent are also provided.

In some aspects and embodiments of the invention described herein, the adenoid cystic carcinoma is recurrent. In some embodiments, the adenoid cystic carcinoma has metastasized.

In some embodiments of the methods described herein, the adenocarcinoma or the adenoid cystic carcinoma comprises high levels of Notch1 intracellular domain (ICD). In some embodiments, the adenocarcinoma has an increased level or an elevated level of Notch1 ICD as compared to a predetermined level of Notch1 ICD. In some embodiments, the adenoid cystic carcinoma comprises high levels of Notch1 ICD as compared to a predetermined level of Notch1 ICD. In some embodiments, the adenoid cystic carcinoma has an increased level or an elevated level of Notch1 ICD as compared to a predetermined level of Notch1 ICD. Thus, the present invention also provides methods of treating an adenocarcinoma or an adenoid cystic carcinoma in a subject comprising: determining if the carcinoma has an increased level or an elevated level of Notch1 ICD as compared to a predetermined level of Notch1 ICD and administering to the subject a therapeutically effective amount of a Notch1-binding agent.

In some embodiments of the methods described herein, the adenocarcinoma comprises a mutation that affects Notch pathway signaling. In some embodiments, the adenocarcinoma comprises a Notch1 mutation. In some embodiments, the adenoid cystic carcinoma comprises a mutation that affects Notch pathway signaling. In some embodiments, the adenoid cystic carcinoma comprises a Notch1 mutation. The present invention also provides methods of treating an adenocarcinoma or an adenoid cystic carcinoma in a subject comprising: determining if the carcinoma comprises a Notch1 mutation and administering to the subject a therapeutically effective amount of a Notch1-binding agent.

In some embodiments of the methods described herein, the adenocarcinoma or the adenoid cystic carcinoma comprises a Notch1 mutation, wherein the Notch1 mutation is a missense, nonsense, or frameshift mutation. In some embodiments, the Notch1 mutation is a frameshift mutation. In some embodiments, the Notch1 mutation is in the heterodimerization (HD) domain. In some embodiments, the Notch1 mutation is in the transactivation domain (TAD). In some embodiments, the Notch1 mutation is in the PEST domain. In some embodiments, the Notch1 mutation is an activating mutation. In some embodiments, the Notch1 mutation increases Notch1 signaling. In some embodiments, the Notch1 mutation increases signaling of the Notch pathway.

In some embodiments of the methods described herein, a mutation produces ligand-independent Notch1 proteolysis and activation. In some embodiments, a mutation removes a C-terminal PEST domain and stabilizes the Notch1 ICD. In some embodiments, a mutation produces an increased level or an elevated level of Notch1 ICD. In some embodiments, the mutation is in the Notch1 gene. In some embodiments, the mutation is in a gene other than the Notch1 gene.

Another aspect of the present invention provides methods of identifying a subject or selecting a subject with an adenocarcinoma or an adenoid cystic carcinoma for treatment with a Notch1-binding agent (e.g., an anti-Notch1 antibody). In some embodiments, a method of identifying a subject or selecting a subject with an adenocarcinoma or an adenoid cystic carcinoma for treatment with an anti-Notch1 antibody comprises determining if the carcinoma has a Notch1 mutation. In some embodiments, a method comprises determining if the carcinoma has a Notch1 mutation, and selecting the subject for treatment with the anti-Notch1 antibody if the carcinoma has a Notch1 mutation. In some embodiments, a method of identifying a subject or selecting a subject with an adenocarcinoma or an adenoid cystic carcinoma for treatment with an anti-Notch1 antibody comprises determining if the carcinoma has a high level or an elevated level of Notch1 ICD. In some embodiments, a method comprises determining if the carcinoma has a high level or an elevated level of Notch1 ICD, and selecting the subject for treatment with the anti-Notch1 antibody if the carcinoma has a high level or an elevated level of Notch1 ICD. In some embodiments, the method comprises administering a therapeutically effective amount of a Notch1-binding agent to the subject.

In some embodiments of the methods described herein, a sample is obtained from the subject. In some embodiments, the sample is a fresh sample, a frozen sample, or formalin-fixed paraffin-embedded (FFPE) sample.

In some embodiments of the methods described herein, a Notch1 mutation is determined by a PCR-based assay, microarray analysis, or nucleic acid sequencing. In some embodiments, the level of Notch1 ICD is determined by an immunohistochemistry assay.

Another aspect of the present invention provides methods of monitoring a subject receiving a Notch1-binding agent (e.g., an anti-Notch1 antibody) for treatment of an adenoid cystic carcinoma. In some embodiments, a method of monitoring a subject receiving an anti-Notch1 antibody for treatment of an adenoid cystic carcinoma comprises: determining the level of lactate dehydrogenase (LDH) in a sample from the subject receiving treatment, and comparing the level of LDH in the sample to a predetermined level of LDH. In some embodiments, the predetermined level of LDH is determined from a sample obtained from the subject prior to treatment.

In some aspects and embodiments of the invention described herein, the Notch1-binding agent is an anti-Notch1 antibody. In some embodiments, the anti-Notch1 antibody specifically binds human Notch1. In some embodiments, the anti-Notch1 antibody specifically binds the extracellular domain of human Notch1. In some embodiments, the anti-Notch1 antibody specifically binds a non-ligand binding membrane proximal region of the extracellular domain of human Notch1. In some embodiments, the non-ligand binding membrane proximal region of the Notch1 receptor comprises SEQ ID NO:2. In some embodiments, the anti-Notch1 antibody specifically binds within SEQ ID NO:2.

In some embodiments of the methods described herein, the anti-Notch1 antibody comprises a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17). In some embodiments, the anti-Notch1 antibody comprises a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20). In some embodiments, the anti-Notch1 antibody comprises a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17), and a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20).

In some embodiments of the methods described herein, the anti-Notch1 antibody comprises: (a) a heavy chain variable region having at least 90% sequence identity to SEQ ID NO:8 or SEQ ID NO:26; and/or (b) a light chain variable region having at least 90% sequence identity to SEQ ID NO:14, SEQ ID NO:32, or SEQ ID NO:38. In some embodiments, the anti-Notch1 antibody comprises: (a) a heavy chain variable region of SEQ ID NO:8 and a light chain variable region of SEQ ID NO:14, (b) a heavy chain variable region of SEQ ID NO:26 and a light chain variable region of SEQ ID NO:32, or (c) a heavy chain variable region of SEQ ID NO:26 and a light chain variable region of SEQ ID NO:38. In some embodiments, the anti-Notch1 antibody comprises a heavy chain variable region of SEQ ID NO:8 and a light chain variable region of SEQ ID NO:14. In some embodiments, the anti-Notch1 antibody comprises a heavy chain variable region of SEQ ID NO:26 and a light chain variable region of SEQ ID NO:32. In some embodiments, the anti-Notch1 antibody comprises a heavy chain variable region of SEQ ID NO:26 and a light chain variable region of SEQ ID NO:38. In some embodiments, the anti-Notch1 antibody comprises the same heavy chain variable region and light chain variable region as an antibody encoded by the plasmid on deposit as ATCC Patent Deposit Designation PTA-9549. In some embodiments, the anti-Notch1 antibody is an antibody encoded by the plasmid on deposit as ATCC Patent Deposit Designation PTA-9549. In some embodiments, the anti-Notch1 antibody is an antibody that comprises the same CDRs as the antibody produced by the hybridoma on deposit as ATCC Patent Deposit Designation PTA-9405. In some embodiments, the anti-Notch1 antibody is a humanized version of the antibody produced by the hybridoma on deposit as ATCC Patent Deposit Designation PTA-9405. In some embodiments, the anti-Notch1 antibody is OMP-52M51. In some embodiments, the anti-Notch1 antibody is OMP-52M51-H4L3.

Compositions comprising a Notch1-binding agent (e.g., an anti-Notch1 antibody) as described herein are further provided for use in the methods described herein. Pharmaceutical compositions comprising a Notch1-binding agent (e.g., an anti-Notch1 antibody) as described herein and a pharmaceutically acceptable vehicle (or carrier) are further provided for use in the methods described herein.

In some embodiments of each of the aforementioned aspects, as well as other aspects and embodiments described elsewhere herein, the methods further comprise administering at least one additional therapeutic agent appropriate for combination therapy. In some embodiments, the additional therapeutic agent is a chemotherapeutic agent. In some embodiments, the additional therapeutic agent is an antibody. In some embodiments, the additional therapeutic agent is an alkylating agent, a nitrosourea, a taxane, a vinca alkaloid, a topoisomerase inhibitor, an antibiotic, a platinum-based agent, a protein kinase inhibitor, or an angiogenesis inhibitor.

Where aspects or embodiments of the invention are described in terms of a Markush group or other grouping alternatives, the present invention encompasses not only the entire group listed as a whole, but also each member of the group individually and all possible subgroups of the main group, and also the main group absent one or more of the group members. The present invention also envisages the explicit exclusion of one or more of any of the group members in the claims invention.

### DESCRIPTION OF THE FIGURES

FIG. 1. Lactate dehydrogenase assay results from a subject with adenoid cystic carcinoma treated with anti-Notch1 antibody OMP-52M51.

FIG. 2. CT scan of liver from a subject with adenoid cystic carcinoma before and after treatment with anti-Notch1 antibody OMP-52M51.

FIG. 3. Notch1 signaling assay.

FIG. 4. Notch1 ICD Immunohistochemistry Assay. FIG. 4A. Positive control tumor OMP-B40. FIG. 4B. Negative control tumor OMP-C11. FIG. 4C. ACC patient tumor.

### DETAILED DESCRIPTION OF THE INVENTION

The present invention provides novel agents, including, but not limited to polypeptides such as antibodies, that bind one or more human Notch receptors, particularly Notch1. The Notch1-binding agents include antagonists of human Notch1. The Notch1-binding agents include agents that inhibit the human Notch pathway. The Notch1-binding agents include agents that inhibit Notch signaling. Related polypeptides and polynucleotides, compositions comprising the Notch1-binding agents, and methods of making the Notch1-binding agents are also provided. Methods of using the Notch1-binding agents, such as methods of treating adenocarcinomas, e.g., adenoid cystic carcinoma, are further provided. Methods of identifying and/or selecting a subject for treatment with a Notch1-binding agent are also provided, as are methods of monitoring a subject receiving treatment with a Notch1-binding agent.

The present invention provides Notch1-binding agents (e.g., antibodies) that specifically bind the extracellular domain of human Notch1. In some embodiments, the Notch1-binding agents (e.g., antibodies) specifically bind a non-ligand binding membrane proximal region of the extracellular domain of human Notch1. The ligand binding region of Notch1, which is necessary and sufficient for ligand binding, has been identified as EGF repeats 11 and 12, suggesting this region of the Notch1 receptor is important in Notch signaling and tumorigenesis (Rebay et al., 1991, Cell, 67:687; Lei et al., 2003, Dev., 130:6411; Hambleton et al., 2004, Structure, 12:2173). Unexpectedly, antibodies that bind outside the ligand binding domain of the extracellular domain of human Notch receptors have been found to inhibit tumor cell growth in vivo (see U.S. Pat. No. 7,919,092 and International Pub. No. WO 2010/005567). Thus, antibodies that bind outside the ligand binding domain of the extracellular domain of one or more of the human Notch receptors—Notch1, Notch2, Notch3, and Notch4—have value as potential cancer therapeutics.

### I. DEFINITIONS

To facilitate an understanding of the present invention, a number of terms and phrases are defined below.

The term “antibody” means an immunoglobulin molecule that recognizes and specifically binds a target, such as a protein, polypeptide, peptide, carbohydrate, polynucleotide, lipid, or combinations of the foregoing through at least one antigen recognition site or antigen-binding site within the variable region(s) of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies, chimeric antibodies, humanized antibodies, human antibodies, fusion proteins comprising an antigen-binding site of an antibody, and any other modified immunoglobulin molecule comprising an antigen-binding site as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy chain constant domains referred to as alpha, delta, epsilon, gamma, and mu, respectively. The different classes of immunoglobulins have different and well known subunit structures and three-dimensional configurations. Antibodies can be naked or conjugated to other molecules including, but not limited to, toxins and radioisotopes.

The term “antibody fragment” refers to a portion of an intact antibody and as used herein refers to the antigenic determining variable regions or the antigen-binding site of an intact antibody. “Antibody fragment” as used herein comprises an antigen-binding site or epitope-binding site. Examples of antibody fragments include, but are not limited to Fab, Fab′, F(ab′)2, and Fv fragments, linear antibodies, single chain antibodies, and multispecific antibodies formed from antibody fragments.

The term “variable region” of an antibody refers to the variable region of the antibody light chain or the variable region of the antibody heavy chain, either alone or in combination. The variable regions of the heavy chain and light chain generally consist of four framework regions connected by three complementarity determining regions (CDRs) (also known as hypervariable regions). The CDRs in each chain are held together in close proximity by the framework regions and, with the CDRs from the other chain, contribute to the formation of the antigen-binding site of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda Md.); and (2) an approach based on crystallographic studies of antigen-antibody complexes (Al-Lazikani et al., 1997, J. Molec. Biol. 273:927-948). In addition, combinations of these two approaches are sometimes used in the art to determine CDRs.

The term “monoclonal antibody” refers to a homogeneous antibody population involved in the highly specific recognition and binding of a single antigenic determinant or epitope. This is in contrast to polyclonal antibodies that typically include a mixture of different antibodies directed against a variety of different antigenic determinants. The term “monoclonal antibody” encompasses both intact and full-length monoclonal antibodies as well as antibody fragments (such as Fab, Fab′, F(ab′)2, Fv fragments), single chain Fv (scFv) mutants, fusion proteins comprising an antibody portion, and any other modified immunoglobulin molecule comprising an antigen-binding site. Furthermore, “monoclonal antibody” refers to such antibodies made by any number of techniques, including but not limited to, hybridoma production, phage selection, recombinant expression, and transgenic animals.

The term “humanized antibody” refers to antibodies that are specific immunoglobulin chains, chimeric immunoglobulins, or fragments thereof that contain minimal non-human (e.g., murine) sequences.

The term “human antibody” means an antibody produced by a human or an antibody having an amino acid sequence corresponding to an antibody produced by a human made using any technique known in the art. This definition of a human antibody includes intact or full-length antibodies, and fragments thereof.

The term “chimeric antibodies” refers to antibodies wherein the amino acid sequence of the immunoglobulin molecule is derived from two or more species. Typically, the variable region of both light and heavy chains corresponds to the variable region of antibodies derived from one species of mammal (e.g., mouse, rat, rabbit, etc.) with the desired binding specificity, affinity, and/or capability while the constant regions are homologous to the sequences in antibodies derived from another species (usually human) to avoid eliciting an immune response in that species.

The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids (often referred to as “linear epitopes”) and noncontiguous amino acids juxtaposed by tertiary folding of a protein (often referred to as “conformation epitopes”). Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

The terms “specifically binds” or “specific binding” mean that a binding agent or an antibody reacts or associates more frequently, more rapidly, with greater duration, with greater affinity, or with some combination of the above to an epitope or protein than with alternative substances, including related and unrelated proteins. In certain embodiments, “specifically binds” means, for instance, that an antibody binds a protein with a K_{D} of about 0.1 mM or less, but more usually less than about 1 μM. In certain embodiments, “specifically binds” means that an antibody binds a protein at times with a K_{D} of at least about 0.1 μM or less, and at other times at least about 0.01 μM or less. Because of the sequence identity between homologous proteins in different species, specific binding can include an antibody that recognizes a particular protein such as Notch1 in more than one species (e.g., mouse Notch1 and human Notch1). It is understood that an antibody or binding moiety that specifically binds a first target may or may not specifically bind a second target. As such, “specific binding” does not necessarily require (although it can include) exclusive binding, i.e. binding a single target. Thus, an antibody may, in certain embodiments, specifically bind more than one target. In certain embodiments, the multiple targets may be bound by the same antigen-binding site on the antibody. For example, an antibody may, in certain instances, comprise two identical antigen-binding sites, each of which specifically binds the same epitope on two or more proteins (e.g., human Notch1 and human Notch3). In certain alternative embodiments, an antibody may be bispecific and comprise at least two antigen-binding sites with differing specificities. By way of non-limiting example, a bispecific antibody may comprise one antigen-binding site that recognizes an epitope on a Notch1 protein, and further comprises a second, different antigen-binding site that recognizes a different epitope on a second protein, such as DLL4. Generally, but not necessarily, reference to “binding” means specific binding.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains (e.g., dimers or multimers).

The terms “polynucleotide” and “nucleic acid” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

“Conditions of high stringency” may be identified by those conditions that: (1) employ low ionic strength and high temperature for washing, for example 15 mM sodium chloride/1.5 mM sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 in 5×SSC (0.75M NaCl, 75 mM sodium citrate) at 42° C.; or (3) employ during hybridization 50% formamide in 5×SSC, 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC and 50% formamide, followed by a high-stringency wash consisting of 0.1×SSC containing EDTA at 55° C.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are known in the art. These include, but are not limited to, BLAST, ALIGN, Megalign, BestFit, and variations thereof. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 residues in length or any integral value therebetween. In some embodiments, identity exists over a longer region than 60-80 residues, such as at least about 90-100 residues, and in some embodiments the sequences are substantially identical over the full length of the sequences being compared, such as the coding region of a nucleotide sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is considered to be a conservative substitution. Preferably, conservative substitutions in the sequences of the polypeptides and antibodies of the invention do not affect or abolish the binding of the polypeptide or antibody containing the amino acid sequence, to the antigen(s), i.e., the Notch protein to which the polypeptide or antibody binds. Methods of identifying nucleotide and amino acid conservative substitutions which do not eliminate antigen binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure.

As used herein, “substantially pure” refers to material which is at least 50% pure (i.e., free from contaminants). In some embodiments, the material is at least 90% pure, at least 95% pure, at least 98% pure, or at least 99% pure.

The terms “tumor” and “neoplasm” refer to any mass of tissue that results from excessive cell growth or proliferation, either benign (non-cancerous) or malignant (cancerous) including pre-cancerous lesions.

The term “metastasis” as used herein refers to the process by which a cancer spreads or transfers from the site of origin to other regions of the body with the development of a similar cancerous lesion at a new location. A “metastatic” or “metastasizing” cell is one that loses adhesive contacts with neighboring cells and migrates (e.g., via the bloodstream or lymph) from the primary site of disease to secondary sites.

The terms “cancer stem cell” and “CSC” and “tumor stem cell” and “tumor initiating cell” and “solid tumor stem cell” and “tumorigenic stem cell” are used interchangeably herein and refer to a population of cells from a tumor that: (1) have extensive proliferative capacity; 2) are capable of asymmetric cell division to generate one or more kinds of differentiated progeny with reduced proliferative or developmental potential; and (3) are capable of symmetric cell divisions for self-renewal or self-maintenance. In some embodiments, these properties confer on the “cancer stem cells” or “tumor initiating cells” the ability to form palpable tumors upon serial transplantation into an immunocompromised host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” or “tumor cell” and grammatical equivalents refer to the total population of cells derived from a tumor or a pre-cancerous lesion, including both non-tumorigenic cells, which comprise the bulk of the tumor cell population, and tumorigenic stem cells (cancer stem cells). As used herein, the term “tumor cell” will be modified by the term “non-tumorigenic” when referring solely to those tumor cells lacking the capacity to renew and differentiate to distinguish those tumor cells from cancer stem cells.

The term “tumorigenic” as used herein refers to the functional features of a cancer stem cell including the properties of self-renewal (giving rise to additional tumorigenic cancer stem cells) and proliferation to generate all other tumor cells (giving rise to differentiated and thus non-tumorigenic tumor cells).

The term “tumorigenicity” as used herein refers to the ability of a random sample of cells from the tumor to form palpable tumors upon serial transplantation into immunocompromised hosts (e.g., mice). This definition also includes enriched and/or isolated populations of cancer stem cells that form palpable tumors upon serial transplantation into immunocompromised hosts (e.g., mice).

The term “subject” refers to any animal (e.g., a mammal), including, but not limited to, humans, non-human primates, canines, felines, rodents, and the like, which is to be the recipient of a particular treatment. The terms “subject” and “patient” are used interchangeably herein. Typically, the terms “subject” and “patient” as used herein are in reference to a human subject.

The term “pharmaceutically acceptable” refers to a product or compound approved (or approvable) by a regulatory agency of the Federal government or a state government or listed in the U.S. Pharmacopeia or other generally recognized pharmacopeia for use in animals, including humans.

The terms “pharmaceutically acceptable excipient, carrier, or adjuvant” or “acceptable pharmaceutical carrier” refer to an excipient, carrier, or adjuvant that can be administered to a subject, together with at least one agent of the present disclosure, and which does not destroy the activity of the agent. The excipient, carrier, or adjuvant should be non-toxic when administered with an agent in doses sufficient to deliver a therapeutic effect. A pharmaceutically acceptable excipient, carrier or adjuvant is generally considered to be an inactive ingredient of any formulation by those of skill in the art and by the FDA.

The phrase “pharmaceutically acceptable vehicle” refers to a diluent, adjuvant, excipient, or carrier with which at least one agent of the present disclosure is administered.

The term “therapeutically effective amount” refers to an amount of a binding agent, antibody, polypeptide, polynucleotide, small organic molecule, or other drug effective to “treat” a disease or disorder in a subject or mammal. In the case of cancer, the therapeutically effective amount of the drug (e.g., an antibody) can reduce the number of cancer cells; reduce the tumor size; inhibit and/or stop cancer cell infiltration into peripheral organs including, for example, the spread of cancer into soft tissue and bone; inhibit and/or stop tumor metastasis; inhibit and/or stop tumor growth; relieve to some extent one or more of the symptoms associated with the cancer; reduce morbidity and mortality; improve quality of life; decrease tumorigenicity, tumorigenic frequency, or tumorigenic capacity of a tumor; reduce the number or frequency of cancer stem cells in a tumor; differentiate tumorigenic cells to a non-tumorigenic state; or a combination of such effects. To the extent the drug prevents growth and/or kills existing cancer cells, it can be referred to as cytostatic and/or cytotoxic.

Terms such as “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent and/or slow the development of a targeted pathologic condition or disorder. Thus, those in need of treatment include those already with the disorder; those prone to have the disorder; and those in whom the disorder is to be prevented. In certain embodiments, a subject is successfully “treated” for cancer according to the methods of the present invention if the patient shows one or more of the following: a reduction in the number of, or complete absence of, cancer or tumor cells; a reduction in the tumor size; inhibition of, or an absence of, cancer or tumor cell infiltration into peripheral organs including, for example, the spread of tumor into soft tissue and bone; inhibition of, or an absence of, tumor metastasis; inhibition of, or an absence of, tumor growth; relief of one or more symptoms associated with the specific cancer; reduced morbidity and mortality; improvement in quality of life; reduction in tumorigenicity, tumorigenic frequency, or tumorigenic capacity of a tumor; reduction in the number or frequency of cancer stem cells in a tumor; reduction in the number or frequency of tumor initiating cells in a tumor; differentiation of tumorigenic cells to a non-tumorigenic state; or some combination of these effects.

As used in the present disclosure and claims, the singular forms “a” “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B, A or B, A (alone) and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. METHODS OF USE AND PHARMACEUTICAL COMPOSITIONS

The present invention provides methods for treating cancer in a subject using a Notch1-binding agent described herein. In some embodiments, a method of treating cancer in a subject comprises administering to the subject a therapeutically effective amount of a Notch1-binding agent. In some embodiments, a method of treating cancer comprises treating an adenocarcinoma in a subject comprising administering to the subject a therapeutically effective amount of a Notch1-binding agent. In some embodiments, a method of treating an adenocarcinoma in a subject comprising administering to the subject a therapeutically effective amount of a Notch1-binding agent. In some embodiments, a method of treating cancer comprises treating an adenoid cystic carcinoma in a subject comprising administering to the subject a therapeutically effective amount of a Notch1-binding agent. In some embodiments, a method of treating an adenoid cystic carcinoma in a subject comprises administering to the subject a therapeutically effective amount of a Notch1-binding agent.

In some embodiments, a method of treating an adenoid cystic carcinoma in a subject, comprises (a) determining if the adenoid cystic carcinoma has an increased level or elevated level of Notch1 ICD as compared to a predetermined level of Notch1 ICD; and (b) administering a therapeutically effective amount of a Notch1-binding agent to the subject. In some embodiments, a method of treating an adenoid cystic carcinoma in a subject, comprises (a) obtaining a sample from the subject, (b) determining the level of Notch1 ICD in the sample; and (c) administering a therapeutically effective amount of a Notch1-binding agent to the subject if the level of Notch1 ICD is increased or elevated as compared to a predetermined level of Notch1 ICD. In some embodiments, a method of treating an adenoid cystic carcinoma in a subject (a) determining if the adenoid cystic carcinoma has a Notch1 mutation, and (b) administering a therapeutically effective amount of a Notch1-binding agent to the subject.

In some embodiments, a method of treating an adenoid cystic carcinoma in a subject, comprises (a) determining if the adenoid cystic carcinoma has an increased level or elevated level of Notch1 ICD as compared to a predetermined level of Notch1 ICD; and (b) administering a therapeutically effective amount of an antibody that specifically binds human Notch1 to the subject; wherein the antibody comprises: a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17), and a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20). In some embodiments, a method of treating an adenoid cystic carcinoma in a subject, comprises (a) determining if the adenoid cystic carcinoma has a Notch1 mutation; and (b) administering a therapeutically effective amount of an antibody that specifically binds human Notch1 to the subject; wherein the antibody comprises a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17), and a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20). In some embodiments, a method of treating an adenoid cystic carcinoma in a subject, comprises administering a therapeutically effective amount of an antibody that specifically binds human Notch1 to the subject; wherein the antibody comprises a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17), and a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20); and wherein the subject has been selected based on the adenoid cystic carcinoma having an increased level or elevated level of Notch1 ICD as compared to a predetermined level of Notch1 ICD. In some embodiments, a method of treating an adenoid cystic carcinoma in a subject, comprises administering a therapeutically effective amount of an antibody that specifically binds human Notch1 to the subject; wherein the antibody comprises a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17), and a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20); and wherein the subject has been selected based on the adenoid cystic carcinoma having a Notch1 mutation.

In some embodiments, a method of inhibiting growth of an adenocarcinoma comprises contacting the adenocarcinoma with an effective amount of a Notch1-binding agent. In some embodiments, a method of inhibiting growth of an adenoid cystic carcinoma comprises contacting the adenoid cystic carcinoma with an effective amount of a Notch1-binding agent. In some embodiments, a method of inhibiting growth of an adenocarcinoma in a subject comprises administering to the subject a therapeutically effective amount of a Notch1-binding agent. In some embodiments, a method of inhibiting growth of an adenoid cystic carcinoma in a subject comprises administering to the subject a therapeutically effective amount of a Notch1-binding agent.

In some embodiments, a method of decreasing the size of an adenoid cystic carcinoma comprises contacting the carcinoma with an effective amount of a Notch1-binding agent. In some embodiments, a method of decreasing the size of an adenoid cystic carcinoma in a subject comprises administering to the subject a therapeutically effective amount of a Notch1-binding agent.

In some embodiments of the methods described herein, the adenoid cystic carcinoma is recurrent. In some embodiments, the adenoid cystic carcinoma has metastasized. In some embodiments, the adenoid cystic carcinoma has metastasized to the lungs, liver, bones, kidney, and/or brain. In some embodiments, the adenoid cystic carcinoma is refractory to certain treatment(s). In some embodiments, the adenoid cystic carcinoma is chemorefractory.

In some embodiments of the methods described herein, the sensitivity of a tumor (e.g., an adenoid cystic carcinoma) to anti-Notch1 antibody OMP-52M51 is predicted by the level of Notch1 ICD expression. A correlation between high levels of Notch1 ICD and the responsiveness of tumors to anti-Notch1 antibody OMP-52M51 can be exploited to improve methods of treating cancer. Selecting cancer patients for treatment with anti-Notch1 antibody OMP-52M51 whose tumors are determined to likely be responsive to treatment based on the level of Notch1 ICD should increase overall therapeutic value. Therapeutic efficacy can also be improved by not selecting cancer patients for OMP-52M51 therapy whose tumors are determined to likely be non-responsive to treatment.

In some embodiments of the methods described herein, the adenocarcinoma comprises a high level of Notch1 ICD. In some embodiments, the adenoid cystic carcinoma comprises a high level of Notch1 ICD. In some embodiments, the adenocarcinoma has an increased level or an elevated level of Notch1 ICD as compared to a predetermined level of Notch1 ICD. In some embodiments, the adenoid cystic carcinoma has an increased level or an elevated level of Notch1 ICD as compared to a predetermined level of Notch1 ICD.

In some embodiments of the methods described herein, the adenocarcinoma comprises a mutation that effects Notch signaling. In some embodiments, the adenoid cystic carcinoma comprises a mutation that effects Notch signaling. In some embodiments, the adenocarcinoma comprises a Notch1 mutation. In some embodiments, the adenoid cystic carcinoma comprises a Notch1 mutation. In some embodiments, the Notch1 mutation is in the heterodimerization (HD) domain of Notch1. In some embodiments, the Notch1 mutation is in the transactivation domain (TAD) of Notch1. In some embodiments, the Notch1 mutation is in the PEST domain of Notch1. In some embodiments, the Notch1 mutation is within amino acids 1570-1736 of Notch1. In some embodiments, the Notch1 mutation is within amino acids 2090-2320 of Notch1. In some embodiments, the Notch1 mutation is within amino acids 2300-2555 of Notch1. In some embodiments, the Notch1 mutation is within amino acids 1570-1736 of SEQ ID NO:41. In some embodiments, the Notch1 mutation is within amino acids 2090-2320 of SEQ ID NO:41. In some embodiments, the Notch1 mutation is within amino acids 2300-2555 of SEQ ID NO:41. In some embodiments, the Notch1 mutation is an activating mutation. In some embodiments, the Notch1 mutation is a missense mutation. In some embodiments, the Notch1 mutation is a nonsense mutation. In some embodiments, the Notch1 mutation is a frameshift mutation. In some embodiments, the Notch1 mutation increases Notch pathway signaling. In some embodiments, the Notch1 mutation increases Notch1 signaling.

In some embodiments, a mutation produces ligand-independent Notch1 proteolysis and activation. In some embodiments, a mutation removes the C-terminal PEST domain and stabilizes the Notch1 ICD. In some embodiments, a mutation produces an increased level or an elevated level of Notch1 ICD in tumor cells. In some embodiments, the mutation is in the Notch1 gene or a Notch1 protein. In some embodiments, the mutation is in a gene or protein other than Notch1. In some embodiments, the mutation is in a gene or protein other than Notch1 wherein Notch1 signaling is increased. In some embodiments, the mutation is in FBXW7 (which is a negative regulator of Notch). In some embodiments, the mutation is a loss-of-function mutation in FBXW7.

In some embodiments, the adenocarcinoma comprises a mutation in p53. In some embodiments, the adenoid cystic carcinoma comprises a mutation in p53. The sequence of p53 is well-known in the art and can be referenced in UniProtKB No. P04637 and GenBank No. NP 000537.3. In some embodiments, the mutation in p53 is within the DNA-binding core domain (DBD). In some embodiments, the mutation in p53 is within amino acids 102-292 of SEQ ID NO:40. In some embodiments, the mutation in p53 is at residue 248 of p53. In some embodiments, the mutation in p53 is at residue 248 of SEQ ID NO:40. In some embodiments, the mutation in p53 at residue 248 of p53 is an arginine to glutamine substitution. In some embodiments, the mutation in p53 is at residue 282 of p53. In some embodiments, the mutation in p53 is at residue 282 of SEQ ID NO:40. In some embodiments, the mutation in p53 at residue 282 is an arginine to tryptophan substitution.

In some embodiments, a method of inhibiting growth of an adenocarcinoma or an adenoid cystic carcinoma comprises contacting the carcinoma with an anti-Notch1 antibody. In some embodiments, a method of treating an adenocarcinoma or an adenoid cystic carcinoma in a subject comprises administering to the subject a therapeutically effective amount of an anti-Notch1 antibody. In some embodiments, a method of treating adenoid cystic carcinoma in a subject comprises: (a) determining if the adenoid cystic carcinoma comprises a Notch1 mutation, and (b) administering to the subject a therapeutically effective amount of a Notch1-binding agent. In some embodiments, a method of treating an adenocarcinoma or an adenoid cystic carcinoma in a subject comprises: (a) determining if the carcinoma has an increased level or an elevated level of Notch1 ICD as compared to a predetermined level of Notch1 ICD, and (b) administering to the subject a therapeutically effective amount of a Notch1-binding agent.

In certain embodiments of the methods described herein, the Notch1-binding agent is an antibody that specifically binds human Notch1 (an anti-Notch1 antibody). In some embodiments, the anti-Notch1 antibody comprises a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17). In some embodiments, the anti-Notch1 antibody comprises a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20). In certain embodiments, the anti-Notch1 antibody comprises a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17), and a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20). In certain embodiments, the anti-Notch1 antibody comprises a heavy chain variable region comprising SEQ ID NO:8 or SEQ ID NO:26. In certain embodiments, the anti-Notch1 antibody further comprises a light chain variable region comprising SEQ ID NO:14, SEQ ID NO:32, or SEQ ID NO:38. In certain embodiments, the anti-Notch1 antibody comprises a heavy chain variable region comprising SEQ ID NO:8 and a light chain variable region comprising SEQ ID NO:14. In certain embodiments, the anti-Notch1 antibody comprises a heavy chain variable region comprising SEQ ID NO:26 and a light chain variable region comprising SEQ ID NO:32. In certain embodiments, the anti-Notch1 antibody comprises a heavy chain variable region comprising SEQ ID NO:26 and a light chain variable region comprising SEQ ID NO:38. In some embodiments, the anti-Notch1 antibody comprises SEQ ID NO:23. In some embodiments, the anti-Notch1 antibody further comprises SEQ ID NO:29 or SEQ ID NO:35. In some embodiments, the anti-Notch1 antibody comprises SEQ ID NO:23 and SEQ ID NO:29. In some embodiments, the anti-Notch1 antibody comprises SEQ ID NO:23 and SEQ ID NO:35. In certain embodiments, the anti-Notch1 antibody comprises the same heavy chain and light chain amino acid sequences as an antibody encoded by a plasmid deposited with ATCC having deposit no. PTA-9549. In certain embodiments, the anti-Notch1 antibody is encoded by the plasmid having ATCC deposit no. PTA-9549 which was deposited with the American Type Culture Collection (ATCC), at 10801 University Boulevard, Manassas, Va., 20110, under the conditions of the Budapest Treaty on Oct. 15, 2008. In certain embodiments, the anti-Notch1 antibody comprises the same CDR sequences as the antibody produced by the hybridoma deposited with ATCC having deposit no. PTA-9405 which was deposited with the American Type Culture Collection (ATCC), at 10801 University Boulevard, Manassas, Va., 20110, under the conditions of the Budapest Treaty on Aug. 7, 2008. In certain embodiments, the anti-Notch1 antibody is a humanized version of the antibody produced by the hybridoma deposited with ATCC having deposit no. PTA-9405. In certain embodiments, the anti-Notch1 antibody is OMP-52M51. In certain embodiments, the anti-Notch1 antibody is a humanized version of OMP-52M51. In certain embodiments, the anti-Notch1 antibody is OMP-52M51-H4L3.

In certain embodiments of the methods described herein, an anti-Notch1 antibody competes for specific binding to human Notch1 with an antibody that comprises the same heavy chain variable region and light chain variable region encoded by the plasmid deposited with ATCC having deposit no. PTA-9549. In certain embodiments, an anti-Notch1 antibody competes for specific binding to human Notch1 with an antibody encoded by the plasmid deposited with ATCC having deposit no. PTA-9549. In certain embodiments, an anti-Notch1 antibody competes for specific binding to human Notch1 with an antibody produced by the hybridoma deposited with ATCC having deposit no. PTA-9405. In certain embodiments, an anti-Notch1 antibody competes for specific binding to human Notch1 with OMP-52M51. In certain embodiments, an anti-Notch1 antibody competes for specific binding to human Notch1 with a humanized version of OMP-52M51. In certain embodiments, an anti-Notch1 antibody competes for specific binding to human Notch1 with OMP-52M51-H4L3.

In another aspect, the invention provides methods for identifying, selecting, and/or stratifying tumors and/or patients with an adenocarcinoma or an adenoid cystic carcinoma that are likely to be responsive (“sensitive”) or non-responsive (“resistant”) to treatment with a Notch1-binding agent. In addition, provided are methods for treating patients with an adenocarcinoma or an adenoid cystic carcinoma who are likely to respond to treatment, are predicted to respond to treatment, and/or have been identified to respond to treatment with a Notch1-binding agent.

In some embodiments, a method of identifying a subject or selecting a subject with an adenocarcinoma or an adenoid cystic carcinoma for treatment with a Notch1-binding agent (e.g., an anti-Notch1 antibody), including but not limited to, each of the Notch1-binding agents described herein, is provided. In some embodiments, a method of selecting a subject with an adenoid cystic carcinoma for treatment with a Notch1-binding agent, comprises (a) determining the level of Notch1 ICD in the adenoid cystic carcinoma; and (b) selecting the subject for treatment with the Notch1-binding agent if the adenoid cystic carcinoma has an increased level or an elevated level of Notch1 ICD as compared to a predetermined level of Notch ICD. In some embodiments, a method of selecting a subject with an adenoid cystic carcinoma for treatment with a Notch1-binding agent, comprises (a) obtaining a sample from the subject, (b) determining the level of Notch1 ICD in the sample; and (c) selecting the subject for treatment with the Notch1-binding agent if the sample has an increased level or an elevated level of Notch1 ICD as compared to a predetermined level of Notch1 ICD. In some embodiments, a method of selecting a subject with an adenoid cystic carcinoma for treatment with a Notch1-binding agent comprises (a) determining if the adenoid cystic carcinoma has a Notch1 mutation, and (b) selecting the subject for treatment with the Notch1-binding agent if the adenoid cystic carcinoma has a Notch1 mutation. In some embodiments, a method of selecting a subject with an adenoid cystic carcinoma for treatment with a Notch1-binding agent comprises (a) obtaining a sample from the subject; (b) determining if the sample has a Notch1 mutation, and (c) selecting the subject for treatment with the Notch1-binding agent if the sample has a Notch1 mutation. In some embodiments, the method comprises administering a therapeutically effective amount of a Notch1-binding agent described herein to the subject.

In some embodiments, a method of selecting a subject with an adenoid cystic carcinoma for treatment with an antibody that specifically binds human Notch1, comprises (a) determining the level of Notch1 ICD in the adenoid cystic carcinoma; (b) selecting the subject for treatment with the antibody if the adenoid cystic carcinoma has an increased level or elevated level of Notch1 ICD as compared to a predetermined level of Notch1 ICD; and (c) administering a therapeutically effective amount of the antibody to the subject; wherein the antibody comprises: a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17), and a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20). In some embodiments, a method of selecting a subject with an adenoid cystic carcinoma for treatment with an antibody that specifically binds human Notch1, comprises (a) determining if the adenoid cystic carcinoma has a Notch1 mutation; (b) selecting the subject for treatment with the antibody if the adenoid cystic carcinoma has a Notch1 mutation; and (c) administering a therapeutically effective amount of the antibody to the subject; wherein the antibody comprises: a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17), and a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20).

In some embodiments of the methods describe herein, the predetermined level of Notch1 ICD is the amount of Notch1 ICD in a normal tissue sample. In some embodiments, the predetermined level of Notch1 ICD is the amount of Notch1 ICD in a cancer or tumor sample without a Notch1 activating mutation. In some embodiments, the predetermined level of Notch1 ICD is the amount of Notch1 ICD in a tissue sample that has an H-score of 1 or less in an immunohistochemistry assay. In some embodiments, the predetermined level of Notch1 ICD is an H-score cut-off level determined using positive and negative controls in an IHC assay.

Methods for determining if a tumor or cancer has a mutation within a specific gene are known by those of skill in the art. Methods for determining if a tumor or cancer has a Notch1 mutation are known by those of skill in the art. For determination at a nucleic acid level, methods include, but are not limited to, PCR-based assays, microarray analyses and nucleotide sequencing (e.g., NextGen sequencing, whole-genome sequencing (WGS)).

Methods for detecting Notch1 in tumor samples are known by those of skill in the art. Methods for detecting Notch1 ICD within tumor samples are provided herein.

In some embodiments of the methods described herein, a sample is obtained from the subject. In some embodiments, the sample is processed to a cell lysate. In some embodiments, the sample is processed to DNA. In some embodiments, the sample is processed to RNA.

In some embodiments of the methods described herein, the sample includes, but is not limited to, any clinically relevant tissue sample, such as a tumor biopsy, a core biopsy tissue sample, a fine needle aspirate, a hair follicle, or a sample of bodily fluid, such as blood, plasma, serum, lymph, ascites fluid, cystic fluid, or urine. In some embodiments, the sample is taken from a patient having an adenocarcinoma or an adenoid cystic carcinoma. In some embodiments, the sample is a primary tumor. In some embodiments, the sample is a metastasis. The sample may be taken from a human, or from non-human mammals such as, mice, rats, non-human primates, canines, felines, ruminants, swine, or sheep. In some embodiments, samples are taken from a subject at multiple time points, for example, before treatment, during treatment, and/or after treatment. In some embodiments, samples are taken from different locations in the subject, for example, a sample from a primary tumor and a sample from a metastasis in a distant location.

In some embodiments of the methods described herein, the sample is a paraffin-embedded fixed tissue sample. In some embodiments, the sample is a formalin-fixed paraffin embedded (FFPE) tissue sample. In some embodiments, the sample is a fresh tissue (e.g., tumor) sample. In some embodiments, the sample is a frozen tissue sample. In some embodiments, the sample is a fresh frozen (FF) tissue (e.g., tumor) sample. In some embodiments, the sample is a cell isolated from a fluid. In some embodiments, the sample comprises circulating tumor cells (CTCs). In some embodiments, the sample is an archival tissue sample. In some embodiments, the sample is an archival tissue sample with known diagnosis, treatment, and/or outcome history. In some embodiments, the sample is a block of tissue. In some embodiments, the sample is dispersed cells. In some embodiments, the sample size is from about 1 cell to about 1×10⁶ cells or more. In some embodiments, the sample size is about 10 cells to about 1×10⁵ cells. In some embodiments, the sample size is about 10 cells to about 10,000 cells. In some embodiments, the sample size is about 10 cells to about 1,000 cells. In some embodiments, the sample size is about 10 cells to about 100 cells. In some embodiments, the sample size is about 1 cell to about 10 cells. In some embodiments, the sample size is a single cell.

In some embodiments, Notch1 expression is analyzed by assessing protein expression as compared to gene expression. Commonly used methods for the analysis of protein expression, include but are not limited to, immunohistochemistry (IHC)-based, antibody-based, and mass spectrometry-based methods. Antibodies, generally monoclonal antibodies, may be used to detect expression of a gene product (e.g., protein). In some embodiments, the antibodies can be detected by direct labeling of the antibodies themselves. In other embodiments, an unlabeled primary antibody is used in conjunction with a labeled secondary antibody.

In some embodiments, Notch1 expression is determined by an assay known to those of skill in the art, including but not limited to, multi-analyte profile test, enzyme-linked immunosorbant assay (ELISA), radioimmunoassay, Western blot assay, immunofluorescent assay, enzyme immunoassay, immunoprecipitation assay, chemiluminescent assay, immunohistochemistry (IHC) assay, dot blot assay, slot blot assay, protein arrays, and FACS. In some embodiments, the level of Notch1 ICD is determined by an IHC assay.

In some embodiments of the methods described herein, the level of Notch1 ICD is determined using an agent that specifically binds Notch1 ICD. In some embodiments, the level of Notch1 ICD is determined using an agent that specifically bind Notch1 ICD and does not bind full-length Notch1. Any molecular entity that displays specific binding to Notch1 ICD can be employed to determine the level of Notch1 ICD protein in a sample. Specific binding agents include, but are not limited to, antibodies, antibody mimetics, and polynucleotides (e.g., aptamers). One of skill understands that the degree of specificity required is determined by the particular assay used to detect Notch1 ICD. In some embodiments, the agent used to detect and/or determine the level of Notch1 ICD is an anti-Notch1 ICD antibody. In some embodiments, the anti-Notch1 ICD antibody is the antibody D3B8 (#4147 Cell Signaling Technology).

In some embodiments, wherein an antibody is used in the assay the antibody is detectably labeled. Examples of detectable substances include various enzymes, prosthetic groups, fluorescent materials, luminescent materials, bioluminescent materials, and radioactive materials. Examples of suitable enzymes include horseradish peroxidase, alkaline phosphatase, 0-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

In some embodiments of the methods described herein, the level of Notch1 ICD is determined using an IHC assay. For example, 4 μm-thick FFPE sections are cut from a tumor sample and mounted on coated glass slides. Tissues are deparaffinized and rehydrated by successively incubating them in xylene, 100% ethanol, 95% ethanol, 70% ethanol, and distilled water for antigen retrieval. Slides are placed into retrieval solution and placed in a decloaker for antigen retrieval. To block endogenous peroxidase activity slides are incubated in hydrogen peroxide and washed in PBS. To block non-specific background staining slides are incubated in a blocker. Slides are incubated with an anti-Notch1 ICD antibody for an appropriate amount of time. Specific binding is detected using a kit including diaminobenzidine (DAB). The sections are counterstained with hematoxylin. In some embodiments, the FFPE sections are mounted on coated glass slides and stained using an automated system, e.g., on a Ventana BenchMark ULTRA instrument using Ventana reagents. In some embodiments, the antibody used in an IHC assay is anti-Notch1 ICD antibody D3B8.

The IHC slides may be analyzed using an automated instrument or evaluated manually by microscope. The staining intensity of each tumor nuclei (0: no expression, 1: weak expression, 2: moderate expression, 3: strong expression) is measured and nuclei of each staining level are counted and a percentage for each type is calculated. The data is combined into a weighted H-score for each tissue section: H-score=[3×(% 3+nuclei)]+[2×(% 2+nuclei)]+[1×(% 1+nuclei)]. Using these parameters, the highest score available is H-score=300. In some embodiments, an H-score of 1 or less is considered negative. In some embodiments, the IHC assay has a cut-off value. In some embodiments, the IHC assay has a cut-off value for specificity. In some embodiments, the IHC assay has a cut-off value for efficacy. In some embodiments, the IHC assay has a cut-off value determined by screening of positive and negative tumor tissues. In some embodiments, the IHC assay has a cut-off value of about 25. In some embodiments, the IHC assay has a cut-off value of about 30, about 40, about 50, about 60, about 70, about 80, about 90, about 100, or about 110, or about 120. In some embodiments, the antibody used in an IHC assay to establish a cut-off value is anti-Notch1 ICD antibody D3B8.

In another aspect, the invention provides a method of decreasing pain in a subject with an adenocarcinoma comprising administering to the subject a therapeutically effective amount of a Notch1-binding agent. In some embodiments, the invention provides a method of decreasing pain in a subject with an adenoid cystic carcinoma comprising administering to the subject a therapeutically effective amount of a Notch1-binding agent are also provided. In some embodiments, the pain is bone pain. In some embodiments, the Notch1-binding agent is an anti-Notch1 antibody described herein.

In some embodiments, the method of treating an adenocarcinoma or an adenoid cystic carcinoma comprises administration of a dose of an anti-Notch1 antibody of about 0.25 mg/kg, about 0.5 mg/kg, about 1.0 mg/kg, about 2.5 mg/kg, about 5 mg/kg, about 10 mg/kg, about 12.5 mg/kg, about 15 mg/kg, or about 20 mg/kg. In some embodiments, the anti-Notch1 antibody is administered once a week, once every two weeks, once every three weeks, or once every four weeks. In some embodiments, the method of treating an adenocarcinoma or an adenoid cystic carcinoma comprises administration of about 1mg/kg once every four weeks. In some embodiments, the method of treating an adenocarcinoma or an adenoid cystic carcinoma comprises administration of about 2.5 mg/kg once every four weeks. In some embodiments, the method of treating an adenocarcinoma or an adenoid cystic carcinoma comprises administration of about 5 mg/kg once every four weeks. In some embodiments, the method of treating an adenocarcinoma or an adenoid cystic carcinoma comprises administration of about 2.5 mg/kg once every three weeks. In some embodiments, the method of treating an adenocarcinoma or an adenoid cystic carcinoma comprises administration of about 5 mg/kg once every three weeks.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

Side effects from therapeutic agents may include, but are not limited to, hives, skin rashes, itching, nausea, vomiting, decreased appetite, diarrhea, chills, fever, fatigue, muscle aches and pain, headaches, low blood pressure, high blood pressure, hypokalemia, low blood counts, bleeding, and cardiac problems.

Thus, one aspect of the present invention is directed to methods of treating an adenocarcinoma or an adenoid cystic carcinoma in a subject comprising administering an anti-Notch1 antibody using an intermittent dosing regimen. As used herein, “intermittent dosing” refers to a dosing regimen using a dosing interval of more than once a week, e.g., dosing once every 2 weeks, once every 3 weeks, once every 4 weeks, etc. In some embodiments, a method for treating an adenocarcinoma or an adenoid cystic carcinoma in a subject comprises administering to the subject an effective dose of an anti-Notch1 antibody according to an intermittent dosing regimen. In some embodiments, a method for treating an adenocarcinoma or an adenoid cystic carcinoma in a subject comprises administering to the subject an effective dose of an anti-Notch1 antibody according to an intermittent dosing regimen, and increasing the therapeutic index of the anti-Notch1 antibody. In some embodiments, the intermittent dosing regimen comprises administering an initial dose of an anti-Notch1 antibody to the subject, and administering subsequent doses of the anti-Notch1 antibody about once every 2 weeks. In some embodiments, the intermittent dosing regimen comprises administering an initial dose of an anti-Notch1 antibody to the subject, and administering subsequent doses of the anti-Notch1 antibody about once every 3 weeks. In some embodiments, the intermittent dosing regimen comprises administering an initial dose of an anti-Notch1 antibody to the subject, and administering subsequent doses of the anti-Notch1 antibody about once every 4 weeks.

In certain embodiments, the method of treating an adenoid cystic carcinoma in a subject comprises: administering to the subject a dose of an anti-Notch1 antibody described herein of about 0.25, 0.5, 1.0, or 2.5 mg/kg once every four weeks. In certain embodiments, the method of treating an adenoid cystic carcinoma in a subject comprises: administering to the subject a dose of an anti-Notch1 antibody described herein of about 2.5, 5.0, or 10.0 mg/kg once every three weeks. In certain embodiments, the method of treating an adenoid cystic carcinoma in a subject comprises: administering to the subject a dose of an anti-Notch1 antibody of about 2.5 mg/kg once every three weeks, wherein the anti-Notch1 antibody comprises a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17), and a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20).

In some embodiments, a dosing regimen may be limited to a specific number of administrations or “cycles”. In some embodiments, the anti-Notch1 antibody is administered for 3, 4, 5, 6, 7, 8, or more cycles. For example, the anti-Notch1 antibody is administered every 3 weeks for 6 cycles, the anti-Notch1 antibody is administered every 4 weeks for 6 cycles, the anti-Notch1 antibody is administered every 3 weeks for 4 cycles, the anti-Notch1 antibody is administered every 4 weeks for 4 cycles, etc. Dosing schedules can be decided upon and subsequently modified by those skilled in the art (e.g., a treating physician).

The choice of a delivery method for doses of the anti-Notch1 antibody may be made according to the ability of the subject to tolerate introduction of the anti-Notch1 antibody into the body. Thus, in any of the aspects and/or embodiments described herein, the administration of the anti-Notch1 antibody may be by intravenous injection. In some embodiments, the administration is by intravenous infusion. In any of the aspects and/or embodiments described herein, the administration of the anti-Notch1 antibody may be by a non-intravenous route.

The present invention further provides pharmaceutical compositions comprising the Notch1-binding agents (e.g., anti-Notch1 antibody) described herein. In certain embodiments, the pharmaceutical compositions further comprise a pharmaceutically acceptable vehicle. In some embodiments, these pharmaceutical compositions find use in inhibiting tumor growth and/or treating cancer in a subject (e.g., a human patient).

In certain embodiments, the pharmaceutical compositions or formulations are prepared for storage and use by combining a purified antibody or agent of the present invention with a pharmaceutically acceptable vehicle (e.g., a carrier or excipient). Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22st Edition, 2012, Pharmaceutical Press, London.)

The pharmaceutical compositions or formulations of the present invention can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous (e.g., injection or infusion), intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid pre-formulation composition containing a homogeneous mixture of a compound of the present invention, or a non-toxic pharmaceutically acceptable salt thereof. The solid pre-formulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The Notch1-binding agents described herein can also be entrapped in microcapsules. Such microcapsules are prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22st Edition, 2012, Pharmaceutical Press, London.

In certain embodiments, a Notch1-binding agent (e.g., an antibody) of the present invention is complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations can be produced. Suitable examples of sustained-release preparations include semi-permeable matrices of solid hydrophobic polymers containing a Notch1-binding agent (e.g., an antibody), where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

In another aspect of the invention, the methods described herein may further comprise administering at least one additional therapeutic agent. An additional therapeutic agent can be administered prior to, concurrently with, and/or subsequently to, administration of the anti-Notch1 antibody. Pharmaceutical compositions comprising an anti-Notch1 antibody and an additional therapeutic agent(s) are also provided. In some embodiments, the at least one additional therapeutic agent comprises 1, 2, 3, or more additional therapeutic agents.

Combination therapy with at least two therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing side effects and/or toxicities. Combination therapy may decrease the likelihood that resistant cancer cells will develop. In some embodiments, combination therapy comprises a therapeutic agent that primarily affects (e.g., inhibits or kills) non-tumorigenic cells and a therapeutic agent that primarily affects (e.g., inhibits or kills) tumorigenic CSCs.

It will be appreciated that the combination of an anti-Notch1 antibody and an additional therapeutic agent may be administered in any order or concurrently. In some embodiments, the anti-Notch1 antibody is administered to subjects that have previously undergone treatment with a second therapeutic agent. In certain other embodiments, the anti-Notch1 antibody and a second therapeutic agent is administered substantially simultaneously or concurrently. For example, a subject may be given an anti-Notch1 antibody while undergoing a course of treatment with a second therapeutic agent (e.g., chemotherapy). In certain embodiments, an anti-Notch1 antibody is administered within 1 year of the treatment with a second therapeutic agent. In certain alternative embodiments, an anti-Notch1 antibody is administered within 10, 8, 6, 4, or 2 months of any treatment with a second therapeutic agent. In certain other embodiments, an anti-Notch1 antibody is administered within 4, 3, 2, or 1 weeks of any treatment with a second therapeutic agent. In some embodiments, an anti-Notch1 antibody is administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

Useful classes of therapeutic agents include, for example, antitubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the additional therapeutic agent is an alkylating agent, an antimetabolite, an antimitotic, a topoisomerase inhibitor, or an angiogenesis inhibitor.

Therapeutic agents that may be administered in combination with the anti-Notch1 antibody include chemotherapeutic agents. Thus, in some embodiments, the method or treatment involves the administration of an anti-Notch1 antibody of the present invention in combination with a chemotherapeutic agent or a mixture of multiple different chemotherapeutic agents. Treatment with an anti-Notch1 antibody can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4th Edition, 2008, M. C. Perry, Editor, Lippincott, Williams & Wilkins, Philadelphia, Pa.

Chemotherapeutic agents useful in the instant invention include, but are not limited to, alkylating agents such as thiotepa and cyclophosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including, for example, tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above.

In certain embodiments, the chemotherapeutic agent is a topoisomerase inhibitor. Topoisomerase inhibitors are chemotherapeutic agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, binblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1.

In some embodiments, an additional therapeutic agent comprises an agent such as a small molecule. For example, treatment can involve the combined administration of an anti-Notch1 antibody of the present invention with a small molecule that acts as an inhibitor against additional tumor-associated proteins including, but not limited to, EGFR, ErbB2, HER2, and/or VEGF. In certain embodiments, the additional therapeutic agent is a small molecule that inhibits a cancer stem cell pathway. In some embodiments, the additional therapeutic agent is a small molecule inhibitor of the Notch pathway. In some embodiments, the additional therapeutic agent is a small molecule inhibitor of the Wnt pathway. In some embodiments, the additional therapeutic agent is a small molecule inhibitor of the BMP pathway. In some embodiments, the additional therapeutic agent is a small molecule that inhibits β-catenin signaling.

In some embodiments, an additional therapeutic agent comprises a biological molecule, such as an antibody. For example, treatment can involve the combined administration of an anti-Notch1 antibody of the present invention with other antibodies against additional tumor-associated proteins including, but not limited to, antibodies that bind EGFR, ErbB2, HER2, and/or VEGF. In certain embodiments, the additional therapeutic agent is an antibody that is an anti-cancer stem cell marker antibody. In some embodiments, the additional therapeutic agent is an antibody that binds an additional component of the Notch pathway. In some embodiments, the additional therapeutic agent is an antibody that binds a component of the Wnt pathway. In certain embodiments, the additional therapeutic agent is an antibody that inhibits a cancer stem cell pathway. In some embodiments, the additional therapeutic agent is an antibody that inhibits the Notch pathway. In some embodiments, the additional therapeutic agent is an antibody that inhibits the Wnt pathway. In some embodiments, the additional therapeutic agent is an antibody that inhibits the BMP pathway. In some embodiments, the additional therapeutic agent is an antibody that inhibits β-catenin signaling. In certain embodiments, the additional therapeutic agent is an antibody that is an angiogenesis inhibitor or modulator (e.g., an anti-VEGF or VEGF receptor antibody). In certain embodiments, the additional therapeutic agent is bevacizumab (AVASTIN), trastuzumab (HERCEPTIN), panitumumab (VECTIBIX), or cetuximab (ERBITUX). Combined administration can include co-administration, either in a single pharmaceutical formulation or using separate formulations, or consecutive administration in either order but generally within a time period such that all active agents can exert their biological activities simultaneously.

Treatment with an anti-Notch1 antibody described herein can include combination treatment with other biologic molecules, such as one or more cytokines (e.g., lymphokines, interleukins, tumor necrosis factors, and/or growth factors) or can be accompanied by surgical removal of tumors, cancer cells, or any other therapy deemed necessary by a treating physician.

In certain embodiments of the methods described herein, the treatment involves the administration of an anti-Notch1 antibody described herein in combination with radiation therapy. Treatment with an anti-Notch1 antibody can occur prior to, concurrently with, or subsequent to administration of radiation therapy. Dosing schedules for such radiation therapy can be determined by the skilled medical practitioner.

In another aspect, the invention provides methods of monitoring a subject receiving treatment with an anti-Notch1 antibody, comprising: determining the level of a biomarker in a sample from the subject receiving treatment, and comparing the level of the biomarker in the sample to a predetermined level of the biomarker. In some embodiments, a decrease in the level of the biomarker in the sample as compared to the predetermined level of the biomarker indicates a positive effect of the treatment. A positive effect of the treatment may include, but is not limited to, a decrease in size of a tumor, a decrease in number of tumors, a decrease in number of metastases, a decrease in pain, a stabilization of tumor size, or stabilization of tumor number, etc. Many cancer types can raise lactate dehydrogenase (LDH) levels, and measuring LDH levels can be helpful in monitoring treatment. Thus, in some embodiments, the subject has an elevated level of LDH as compared to a predetermined level or a normal level. In some embodiments, a method of monitoring a subject receiving an anti-Notch1 antibody for treatment of an adenoid cystic carcinoma, comprises: determining the level of lactate dehydrogenase (LDH) in a sample from the subject after treatment and comparing the level of LDH in the sample to a predetermined level of LDH from the subject prior to treatment. In some embodiments, a method of monitoring a subject receiving an anti-Notch1 antibody for treatment of an adenoid cystic carcinoma, comprises: obtaining a sample from the subject receiving treatment, determining the level of LDH in the sample, and comparing the level of LDH in the sample to a predetermined level of LDH from the subject prior to treatment. In some embodiments, a method of monitoring a subject receiving an anti-Notch1 antibody for treatment of an adenoid cystic carcinoma, comprises: obtaining a sample from the subject receiving treatment, determining the level of LDH in the sample, and comparing the level of LDH in the sample to a predetermined level of LDH from the subject prior to treatment, wherein a decrease in the level of LDH indicates a positive effect of the treatment. In some embodiments, the sample is blood, serum, or plasma.

### III. NOTCH1 BINDING AGENTS

The present invention provides agents that specifically bind human Notch1, compositions comprising those binding agents, and methods for using those binding agent to treat cancer. In certain embodiments, the present invention provides agents that bind Notch1 and methods of using the binding agents to treat adenocarcinomas. In certain embodiments, the Notch1-binding agent inhibits adenocarcinoma growth. In certain embodiments, the Notch1-binding agent is used to treat adenoid cystic carcinoma. In certain embodiments, the Notch1-binding agent inhibits adenoid cystic carcinoma growth. In certain embodiments, the Notch1-binding agent is an antibody that specifically binds human Notch1 (anti-Notch1 antibody). In some embodiments, the anti-Notch1 antibody specifically binds to the extracellular domain of human Notch1. In some embodiments, the anti-Notch1 antibody specifically binds a non-ligand binding membrane proximal region of the extracellular domain of human Notch1. In some embodiments, the anti-Notch1 antibody binds a region of human Notch1 comprising about amino acid 1427 to about amino acid 1732. In some embodiments, the anti-Notch1 antibody binds a region comprising SEQ ID NO:2. In some embodiments, the anti-Notch1 antibody specifically binds a region within SEQ ID NO:2. In some embodiments, the anti-Notch1 antibody specifically binds an epitope within a region comprising SEQ ID NO:2.

In certain embodiments, the antibody that specifically binds human Notch1 comprises one, two, three, four, five and/or six of the CDRs of antibody OMP-52M51 (see Table 1). In some embodiments, the antibody comprises one or more of the CDRs of OMP-52M51, two or more of the CDRs of OMP-52M51, three or more of the CDRs of OMP-52M51, four or more of the CDRs of OMP-52M51, five or more of the CDRs of OMP-52M51, or all six of the CDRs or OMP-52M51. In some embodiments, the antibody comprises CDRs with up to four (i.e., 0, 1, 2, 3, or 4) amino acid substitutions per CDR. In certain embodiments, the heavy chain CDR(s) are contained within a heavy chain variable region. In certain embodiments, the light chain CDR(s) are contained within a light chain variable region.

TABLE 1

| | OMP-52M51 |
|---|---|
| HC CDR1 | RGYWIE |
| | (SEQ ID NO: 15) |
| HC CDR2 | QILPGTGRTNYNEKFKG |
| | (SEQ ID NO: 16) |
| HC CDR3 | FDGNYGYYAMDY |
| | (SEQ ID NO: 17) |
| LC CDR1 | RSSTGAVTTSNYAN |
| | (SEQ ID NO: 18) |
| LC CDR2 | GTNNRAP |
| | (SEQ ID NO: 19) |
| LC CDR3 | ALWYSNHWVFGGGTKL |
| | (SEQ ID NO: 20) |

In certain embodiments, the antibody that binds human Notch1 comprises (a) a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17); and/or (b) a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20). In some embodiments, the antibody that binds human Notch1 comprises a heavy chain variable region comprising: (a) a heavy chain CDR1 comprising RGYWIE (SEQ ID NO:15), or a variant thereof comprising 1, 2, 3, or 4 amino acid substitutions; (b) a heavy chain CDR2 comprising QILPGTGRTNYNEKFKG (SEQ ID NO:16), or a variant thereof comprising 1, 2, 3, or 4 amino acid substitutions; and (c) a heavy chain CDR3 comprising FDGNYGYYAMDY (SEQ ID NO:17), or a variant thereof comprising 1, 2, 3, or 4 amino acid substitutions. In other embodiments, the antibody that binds human Notch1 comprises (or further comprises) a light chain variable region comprising: (a) a light chain CDR1 comprising RSSTGAVTTSNYAN (SEQ ID NO:18), or a variant thereof comprising 1, 2, 3, or 4 amino acid substitutions; (b) a light chain CDR2 comprising GTNNRAP (SEQ ID NO:19), or a variant thereof comprising 1, 2, 3, or 4 amino acid substitutions; and (c) a light chain CDR3 comprising ALWYSNHWVFGGGTKL (SEQ ID NO:20), or a variant thereof comprising 1, 2, 3, or 4 amino acid substitutions. In some embodiments, the amino acid substitutions are conservative amino acid substitutions.

In some embodiments, the antibody that binds human Notch1 comprises a heavy chain variable region having at least about 90% sequence identity to SEQ ID NO:8, and/or a light chain variable region having at least about 90% sequence identity to SEQ ID NO:14. In some embodiments, the antibody that binds human Notch1 comprises a heavy chain variable region having at least about 95%, at least about 97%, or at least about 99% sequence identity to SEQ ID NO:8, and/or a light chain variable region having at least about 95%, at least about 97%, or at least about 99% sequence identity to SEQ ID NO:14. In some embodiments, the antibody that binds human Notch1 comprises a heavy chain variable region having at least about 95% sequence identity to SEQ ID NO:8, and/or a light chain variable region having at least about 95% sequence identity to SEQ ID NO:14. In some embodiments, the antibody that binds human Notch1 comprises a heavy chain variable region comprising SEQ ID NO:8 and/or a light chain variable region comprising SEQ ID NO:14. In some embodiments, the antibody that binds human Notch1 comprises a heavy chain variable region comprising SEQ ID NO:8 and a light chain variable region comprising SEQ ID NO:14. In some embodiments, the antibody is a monoclonal antibody or an antibody fragment.

In some embodiments, the antibody that binds human Notch1 comprises a heavy chain variable region having at least about 90% sequence identity to SEQ ID NO:26, and/or a light chain variable region having at least about 90% sequence identity to SEQ ID NO:32. In some embodiments, the antibody that binds human Notch1 comprises a heavy chain variable region having at least about 95%, at least about 97%, or at least about 99% sequence identity to SEQ ID NO:26, and/or a light chain variable region having at least about 95%, at least about 97%, or at least about 99% sequence identity to SEQ ID NO:32. In some embodiments, the antibody that binds human Notch1 comprises a heavy chain variable region having at least about 95% sequence identity to SEQ ID NO:26, and/or a light chain variable region having at least about 95% sequence identity to SEQ ID NO:32. In some embodiments, the antibody that binds human Notch1 comprises a heavy chain variable region comprising SEQ ID NO:26 and/or a light chain variable region comprising SEQ ID NO:32. In some embodiments, the antibody that binds human Notch1 comprises a heavy chain variable region comprising SEQ ID NO:26 and a light chain variable region comprising SEQ ID NO:32. In some embodiments, the antibody is a monoclonal antibody or antibody fragment.

In some embodiments, the antibody that binds human Notch1 comprises a heavy chain variable region having at least about 90% sequence identity to SEQ ID NO:26, and/or a light chain variable region having at least about 90% sequence identity to SEQ ID NO:38. In some embodiments, the antibody that binds human Notch1 comprises a heavy chain variable region having at least about 95%, at least about 97%, or at least about 99% sequence identity to SEQ ID NO:26, and/or a light chain variable region having at least about 95%, at least about 97%, or at least about 99% sequence identity to SEQ ID NO:38. In some embodiments, the antibody that binds human Notch1 comprises a heavy chain variable region having at least about 95% sequence identity to SEQ ID NO:26, and/or a light chain variable region having at least about 95% sequence identity to SEQ ID NO:38. In some embodiments, the antibody that binds human Notch1 comprises a heavy chain variable region comprising SEQ ID NO:26 and/or a light chain variable region comprising SEQ ID NO:38. In some embodiments, the antibody that binds human Notch1 comprises a heavy chain variable region comprising SEQ ID NO:26 and a light chain variable region comprising SEQ ID NO:38. In some embodiments, the antibody is a monoclonal antibody or antibody fragment.

In some embodiments, the Notch1-binding agent is an antibody, OMP-52M51 (also referred to as 52M51), produced by the hybridoma cell line deposited with American Type Culture Collection (ATCC), 10801 University Boulevard, Manassas, Va., USA, under the conditions of the Budapest Treaty on Aug. 7, 2008 and assigned number PTA-9405. In some embodiments, the antibody is a humanized version of OMP-52M51. In some embodiments, the antibody is a humanized version of OMP-52M51, “OMP-52M51-H4L3”, as encoded by the plasmid deposited with ATCC, 10801 University Boulevard, Manassas, Va., USA, under the conditions of the Budapest Treaty on Oct. 15, 2008 and assigned number PTA-9549. In some embodiments, the antibody is a humanized version of OMP-52M51, “OMP-52M51-H4L4”. In some embodiments, the invention provides an antibody that binds the same epitope as the epitope to which antibody OMP-52M51 binds. In other embodiments, the invention provides an antibody that competes with any of the antibodies as described in the aforementioned embodiments and/or aspects, as well as other aspects/embodiments described elsewhere herein, for specific binding to a non-ligand binding membrane proximal region of the extracellular domain of human Notch1.

The invention provides a variety of polypeptides, including but not limited to, antibodies and fragments of antibodies. In certain embodiments, the polypeptide is isolated. In some embodiments, the polypeptide is substantially pure.

In certain embodiments, the polypeptides of the present invention can be recombinant polypeptides, natural polypeptides, or synthetic polypeptides comprising the sequence of SEQ ID NO:4, SEQ ID NO:6, SEQ ID NO:8, SEQ ID NO:10, SEQ ID NO:12, SEQ ID NO:14, SEQ ID NO:22, SEQ ID NO:23, SEQ ID NO:25 SEQ ID NO:26, SEQ ID NO:28, SEQ ID NO: 29, SEQ ID NO:31, SEQ ID NO:32, SEQ ID NO:34, SEQ ID NO:35, SEQ ID NO:37, or SEQ ID NO:38 (with or without the signal/leader sequences). In some embodiments, the polypeptides comprise the heavy chain and/or the light chain provided in SEQ ID NO:4, SEQ ID NO:10, SEQ ID NO:22, SEQ ID NO:23, SEQ ID NO:28, SEQ ID NO:29, SEQ ID NO:34, and/or SEQ ID NO:35, respectively (with or without the signal/leader signal sequences). In certain embodiments, the polypeptide is an antibody. In certain embodiments, the polypeptide specifically binds human Notch1. In certain embodiments, the polypeptide specifically binds the extracellular domain of human Notch1. In certain embodiments, the polypeptide specifically binds a non-ligand binding membrane proximal region of the extracellular domain of human Notch1. In certain embodiments, the polypeptide comprises a heavy chain variable region sequence comprising SEQ ID NO:8 and a light chain variable region sequence comprising SEQ ID NO:14. In certain embodiments, the polypeptide comprises a heavy chain variable region sequence comprising SEQ ID NO:26 and a light chain variable region sequence comprising SEQ ID NO:32. In certain embodiments, the polypeptide comprises a heavy chain variable region sequence comprising SEQ ID NO:26 and a light chain variable region sequence comprising SEQ ID NO:38. In certain embodiments, the polypeptide is an antibody.

The polypeptides of the present invention may comprise polypeptides of SEQ ID NO:8 as well as polypeptides that have at least 90% sequence identity to SEQ ID NO:8 and at least 95% sequence identity to SEQ ID NO:8, and in still other embodiments, polypeptides that have at least 96%, 97%, 98%, or 99% sequence identity to SEQ ID NO:8. The polypeptides of the present invention may comprise polypeptides of SEQ ID NO:14 as well as polypeptides that have at least 90% sequence identity to SEQ ID NO:14 and at least 95% sequence identity to SEQ ID NO:14, and in still other embodiments, polypeptides which have at least 96%, 97%, 98%, or 99% sequence identity to SEQ ID NO:14.

The polypeptides of the present invention may comprise polypeptides of SEQ ID NO:26 as well as polypeptides that have at least 90% sequence identity to SEQ ID NO:26 and at least 95% sequence identity to SEQ ID NO:26, and in still other embodiments, polypeptides that have at least 96%, 97%, 98%, or 99% sequence identity to SEQ ID NO:26. The polypeptides of the present invention may comprise polypeptides of SEQ ID NO:32 as well as polypeptides that have at least 90% sequence identity to SEQ ID NO:32 and at least 95% sequence identity to SEQ ID NO:32, and in still other embodiments, polypeptides that have at least 96%, 97%, 98%, or 99% sequence identity to SEQ ID NO:32. The polypeptides of the present invention may comprise polypeptides of SEQ ID NO:38 as well as polypeptides that have at least 90% sequence identity to SEQ ID NO:38 and at least 95% sequence identity to SEQ ID NO:38, and in still other embodiments, polypeptides that have at least 96%, 97%, 98%, or 99% sequence identity to SEQ ID NO:38.

In certain embodiments, the Notch1-binding agent (e.g., an antibody) binds Notch1 and modulates Notch1 activity. In some embodiments, the Notch1-binding agent is an antagonist and modulates Notch1 activity.

In certain embodiments, the Notch1-binding agent (e.g., an antibody) is an antagonist of Notch1 and inhibits Notch1 activity. In certain embodiments, the Notch1-binding agent inhibits at least about 10%, at least about 20%, at least about 30%, at least about 50%, at least about 75%, at least about 90%, or about 100% of the activity of the bound human Notch1. In some embodiments, the Notch1-binding agent inhibits activity of a mutated Notch1. In some embodiments, the Notch1-binding agent inhibits activity of a constitutively activated Notch1. In some embodiments, the mutated Notch1 is in an adenocarcinoma. In certain embodiments, the mutated Notch1 is in an adenoid cystic carcinoma.

In certain embodiments, the Notch1-binding agent (e.g., an antibody) inhibits Notch signaling. It is understood that a Notch1-binding agent that inhibits Notch signaling may, in certain embodiments, inhibit signaling by one or more Notchs, but not necessarily inhibit signaling by all Notchs. In certain alternative embodiments, signaling by all human Notchs may be inhibited. In certain embodiments, signaling by one or more Notchs selected from the group consisting of Notch1, Notch2, Notch3 and Notch4 is inhibited. In certain embodiments, the inhibition of Notch signaling by a Notch1-binding agent is a reduction in the level of Notch1 signaling of at least about 10%, at least about 25%, at least about 50%, at least about 75%, at least about 90%, or at least about 95%.

In certain embodiments, the Notch1-binding agent (e.g., an antibody) inhibits Notch activation. It is understood that a Notch1-binding agent that inhibits Notch activation may, in certain embodiments, inhibit activation of one or more Notchs, but not necessarily inhibit activation of all Notchs. In certain alternative embodiments, activation of all human Notchs may be inhibited. In certain embodiments, activation of one or more Notchs selected from the group consisting of Notch1, Notch2, Notch3, and Notch4 is inhibited. In certain embodiments, the inhibition of Notch activation by a Notch1-binding agent is a reduction in the level of Notch1 activation of at least about 10%, at least about 25%, at least about 50%, at least about 75%, at least about 90%, or at least about 95%.

In vivo and in vitro assays for determining whether a Notch1-binding agent (or candidate Notch1-binding agent) inhibits Notch activation are known in the art. In some embodiments, a cell-based, luciferase reporter assay utilizing a TCF/Luc reporter vector containing multiple copies of the TCF-binding domain upstream of a firefly luciferase reporter gene may be used to measure Notch signaling levels in vitro. In other embodiments, a cell-based, luciferase reporter assay utilizing a CBF/Luc reporter vector containing multiple copies of the CBF-binding domain upstream of a firefly luciferase reporter gene may be used. The level of Notch activation induced by a Notch ligand in the presence of a Notch1-binding agent is compared to the level of Notch activation induced by a Notch ligand in the absence of a Notch1-binding agent.

In certain embodiments, the Notch1-binding agents (e.g., antibodies) have one or more of the following effects: inhibit proliferation of cancer cells, inhibit cancer cell growth, prevent or reduce metastasis of cancer cells, reduce the frequency of cancer stem cells in a tumor or cancer, trigger cell death of cancer cells (e.g., by apoptosis), reduce the tumorigenicity of cancer cells by reducing the frequency of cancer stem cells in the cancer cell population, differentiate tumorigenic cells to a non-tumorigenic state, or increase survival time of a patient.

In certain embodiments, the Notch1-binding agents (e.g., antibodies) are capable of inhibiting cancer cell growth. In certain embodiments, the Notch1-binding agents are capable of inhibiting growth of cancer cells in vitro (e.g., contacting cancer cells with an antibody in vitro). In certain embodiments, the Notch1-binding agents are capable of inhibiting cancer growth in vivo (e.g., in a xenograft mouse model and/or in a human having cancer).

In certain embodiments, the Notch1-binding agents (e.g., antibodies) are capable of reducing the tumorigenicity of an adenocarcinoma. In certain embodiments, the Notch1-binding agents (e.g., antibodies) are capable of reducing the tumorigenicity of an adenoid cystic carcinoma. In certain embodiments, the Notch1-binding agent or antibody is capable of reducing the tumorigenicity of a cancer comprising cancer stem cells in an animal model, such as a mouse xenograft model. In some embodiments, the Notch1-binding agent is capable of reducing the tumorigenicity of a cancer by reducing the frequency of cancer stem cells in the cancer. In certain embodiments, the number or frequency of cancer stem cells in a cancer is reduced by at least about two-fold, about three-fold, about five-fold, about ten-fold, about 50-fold, about 100-fold, or about 1000-fold. In certain embodiments, the reduction in the frequency of cancer stem cells is determined by a limiting dilution assay (LDA) using an animal model. Examples and guidance regarding the use of limiting dilution assays to determine a reduction in the number or frequency of cancer stem cells in a tumor can be found, e.g., in International Pub. No. WO 2008/042236 and U.S. Patent Pub. Nos. 2008/0064049 and 2008/0178305.

In certain embodiments, Notch1-binding agents or antibodies mediate cell death of a cell expressing Notch1 via antibody-dependent cellular cytotoxicity (ADCC). ADCC involves cell lysis by effector cells that recognize the Fc portion of an antibody. Many lymphocytes, monocytes, tissue macrophages, granulocytes and eosinophils, for example, have Fc receptors and can mediate cytolysis.

In certain embodiments, the anti-Notch1 antibody binds human Notch1 with a dissociation constant (K_{D}) of about 1 μM or less, about 100 nM or less, about 40 nM or less, about 20 nM or less, about 10 nM or less, about 1 nM or less, about 0.5 nM or less, or about 0.1 nM or less. In certain embodiments, the anti-Notch1 antibody binds human Notch1 with a K_{D} of about 1 nM or less. In certain embodiments, the anti-Notch1 antibody binds human Notch1 with a K_{D} of about 0.8 nM or less. In certain embodiments, the anti-Notch1 antibody binds human Notch1 with a K_{D} of about 0.6 nM or less. In certain embodiments, the anti-Notch1 antibody binds human Notch1 with a K_{D} of about 0.5 nM or less. In certain embodiments, the anti-Notch1 antibody binds human Notch1 with a K_{D} of about 0.4 nM or less. In certain embodiments, the anti-Notch1 antibody binds human Notch1 with a K_{D} of about 0.3 nM or less. In some embodiments, the K_{D} is measured by surface plasmon resonance. In some embodiments, the dissociation constant of the antibody to Notch1 is the dissociation constant determined using a Notch fusion protein comprising a Notch1 extracellular domain (e.g., a Notch1 ECD-Fc fusion protein) immobilized on a Biacore chip.

In certain embodiments, the anti-Notch1 antibody binds human Notch1 with a half maximal effective concentration (EC₅₀) of about 1 μM or less, about 100 nM or less, about 40 nM or less, about 20 nM or less, about 10 nM or less, or about 1 nM or less. In certain embodiments, the anti-Notch1 antibody binds human Notch1 with an EC₅₀ of about 40 nM or less, about 20 nM or less, about 10 nM or less, or about 1 nM or less.

In some embodiments, the anti-Notch1 antibody is a recombinant antibody. In some embodiments, the anti-Notch1 antibody is a chimeric antibody. In certain embodiments, the anti-Notch1 antibody is an IgG antibody. In some embodiments, the anti-Notch1 antibody is an IgG1 antibody. In some embodiments, the anti-Notch1 antibody is an IgG2 antibody. In certain embodiments, the anti-Notch1 antibody is a monoclonal antibody. In certain embodiments, the anti-Notch1 antibody is a humanized antibody. In certain embodiments, the anti-Notch1 antibody is a human antibody. In certain embodiments, the anti-Notch1 antibody is an antibody fragment comprising an antigen-binding site.

In some embodiments, the anti-Notch1 antibodies are polyclonal antibodies. Polyclonal antibodies can be prepared by any known method. In some embodiments, polyclonal antibodies are prepared by immunizing an animal (e.g., a rabbit, rat, mouse, goat, donkey) by multiple subcutaneous or intraperitoneal injections of the relevant antigen (e.g., a purified peptide fragment, full-length recombinant protein, fusion protein, etc.). The antigen can be optionally conjugated to a carrier protein such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from blood, ascites and the like, of the immunized animal. Polyclonal antibodies can be purified from serum or ascites according to standard methods in the art including, but not limited to, affinity chromatography, ion-exchange chromatography, gel electrophoresis, and dialysis.

In some embodiments, the anti-Notch1 antibodies are monoclonal antibodies. In some embodiments, monoclonal antibodies are prepared using hybridoma methods known to one of skill in the art. Using a hybridoma method, a mouse, hamster, or other appropriate host animal, is immunized as described above to elicit lymphocytes to produce antibodies that will specifically bind the immunizing antigen. In some embodiments, lymphocytes are immunized in vitro. In some embodiments, the immunizing antigen (e.g., a Notch protein) is a human protein or a portion thereof. In some embodiments, the immunizing antigen (e.g., a Notch protein) is a mouse protein or a portion thereof. In some embodiments, the immunizing antigen is an extracellular domain of a human Notch protein. In some embodiments, the immunizing antigen is an extracellular domain of a mouse Notch protein. In some embodiments, a mouse is immunized with a human antigen. In some embodiments, a mouse is immunized with a mouse antigen.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed against a target antigen may be identified by a variety of techniques including, but not limited to, immunoprecipitation, immunoblotting, and in vitro binding assays (e.g., flow cytometry, enzyme-linked immunosorbant assay (ELISA), or radioimmunoassay (RIA)). The hybridomas can be propagated either in vitro in tissue culture using standard methods or in vivo as ascites in a host animal. The monoclonal antibodies can be purified from the culture medium or ascites fluid according to standard methods in the art including, but not limited to, affinity chromatography, ion-exchange chromatography, gel electrophoresis, and dialysis.

In some embodiments, monoclonal antibodies can be made using recombinant DNA techniques as known to one skilled in the art. In some embodiments, the polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional techniques. The isolated polynucleotides encoding the heavy chains and light chains are cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins. In certain embodiments, recombinant monoclonal antibodies, or fragments thereof, can be isolated from phage display libraries expressing variable domain regions or CDRs of a desired species.

The polynucleotide(s) encoding a monoclonal antibody can be modified, for example, by using recombinant DNA technology to generate alternative antibodies. In some embodiments, the constant domains of the light and heavy chains of, for example, a mouse monoclonal antibody can be substituted for those regions of, for example, a human antibody to generate a chimeric antibody or for a non-immunoglobulin polypeptide to generate a fusion antibody. In some embodiments, the constant regions are truncated or removed to generate the desired antibody fragment of a monoclonal antibody. In some embodiments, site-directed or high-density mutagenesis of the variable region can be used to optimize specificity, affinity, and/or other biological characteristics of a monoclonal antibody. In some embodiments, site-directed mutagenesis of the CDRs can be used to optimize specificity, affinity, and/or other biological characteristics of a monoclonal antibody.

In some embodiments, the anti-Notch1 antibody is a humanized antibody. Typically, humanized antibodies are human immunoglobulins in which residues from the CDRs are replaced by residues from a CDR of a non-human species (e.g., mouse, rat, rabbit, hamster, etc.) that have the desired specificity, affinity, and/or binding capability using methods known to one skilled in the art. In some embodiments, the Fv framework region residues of a human immunoglobulin are replaced with the corresponding residues in an antibody from a non-human species that has the desired specificity, affinity, and/or binding capability. In some embodiments, a humanized antibody can be further modified by the substitution of additional residues either in the Fv framework region and/or within the replaced non-human residues to refine and optimize antibody specificity, affinity, and/or capability. In general, a humanized antibody will comprise substantially all of at least one, and typically two or three, variable domain regions containing all, or substantially all, of the CDRs that correspond to the non-human immunoglobulin whereas all, or substantially all, of the framework regions are those of a human immunoglobulin sequence. In some embodiments, a humanized antibody can also comprise at least a portion of an immunoglobulin constant region or domain (Fc), typically that of a human immunoglobulin. In certain embodiments, such humanized antibodies are used therapeutically because they may reduce antigenicity and HAMA (human anti-mouse antibody) responses when administered to a human subject. One skilled in the art would be able to obtain a functional humanized antibody with reduced immunogenicity following known techniques.

In certain embodiments, the anti-Notch1 antibody is a human antibody. Human antibodies can be directly prepared using various techniques known in the art. In some embodiments, human antibodies may be generated from immortalized human B lymphocytes immunized in vitro or from lymphocytes isolated from an immunized individual. In either case, cells that produce an antibody directed against a target antigen can be generated and isolated.

In some embodiments, a human antibody can be selected from a phage library, where that phage library expresses human antibodies. Phage display technology can be used to produce human antibodies and antibody fragments in vitro from immunoglobulin variable domain gene repertoires from unimmunized donors. Various techniques for the generation and use of antibody phage libraries are well-known in the art.

Once antibodies are identified, affinity maturation strategies known in the art, including but not limited to, chain shuffling and site-directed mutagenesis, may be employed to generate high affinity human antibodies.

In some embodiments, human antibodies can be made in transgenic mice that contain human immunoglobulin loci. Upon immunization these mice are capable of producing the full repertoire of human antibodies in the absence of endogenous immunoglobulin production.

In certain embodiments, the anti-Notch1 antibody is a bispecific antibody. Bispecific antibodies are capable of specifically recognizing and binding to at least two different epitopes. The different epitopes can either be within the same molecule or on different molecules. In some embodiments, the antibodies can specifically recognize and bind a first antigen target, (e.g., Notch1) as well as a second antigen target, such as an effector molecule on a leukocyte (e.g., CD2, CD3, CD28, or B7) or a Fc receptor (e.g., CD64, CD32, or CD16) so as to focus cellular defense mechanisms to the cell expressing the first antigen target. In some embodiments, the antibodies can be used to direct cytotoxic agents to cells which express a particular target antigen, such as a Notch protein. These antibodies possess an antigen-binding arm and an arm which binds a cytotoxic agent or a radionuclide chelator, such as EOTUBE, DPTA, DOTA, or TETA. In certain embodiments, the antibodies can be used to affect angiogenesis. In certain embodiments, the bispecific antibody specifically binds Notch1, as well as VEGF. In certain embodiments, the bispecific antibody specifically binds Notch1, as well as a Notch ligand (e.g., DLL4, Jagged1 or Jagged2), or at least one other Notch receptor selected from the group consisting of Notch2, Notch3, and Notch4.

Techniques for making bispecific antibodies are known by those skilled in the art, see for example, Millstein et al., 1983, Nature, 305:537-539; Brennan et al., 1985, Science, 229:81; Suresh et al, 1986, Methods in Enzymol., 121:120; Traunecker et al., 1991, EMBO J., 10:3655-3659; Shalaby et al., 1992, J. Exp. Med., 175:217-225; Kostelny et al., 1992, J. Immunol., 148:1547-1553; Gruber et al., 1994, J. Immunol., 152:5368; U.S. Pat. No. 5,731,168, and U.S. Patent Publication No. 2011/0123532. Bispecific antibodies can be intact antibodies or antibody fragments. Antibodies with more than two valencies are also contemplated. For example, trispecific antibodies can be prepared (Tutt et al., 1991, J. Immunol., 147:60). Thus, in certain embodiments the antibodies to Notch1 are multispecific.

In certain embodiments, the anti-Notch1 antibodies described herein may be monospecific. For example, in certain embodiments, each of the one or more antigen-binding sites that an antibody contains is capable of binding (or binds) a homologous epitope on Notch1.

In certain embodiments, the anti-Notch1 antibody is an antibody fragment. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced recombinantly. In some embodiments, antibody fragments include Fv or single chain Fv (scFv) fragments. Fab, Fv, and scFv antibody fragments can be expressed in, and secreted from, E. coli or other host cells, allowing for the production of large amounts of these fragments. In some embodiments, antibody fragments are isolated from antibody phage libraries as discussed herein. For example, methods can be used for the construction of Fab expression libraries to allow rapid and effective identification of monoclonal Fab fragments with the desired specificity for Notch1, or derivatives, fragments, analogs or homologs thereof. In some embodiments, antibody fragments are linear antibody fragments. In certain embodiments, antibody fragments are monospecific or bispecific. In certain embodiments, the anti-Notch1 antibody is a scFv. Various techniques can be used for the production of single-chain antibodies specific to Notch1.

It can further be desirable, especially in the case of antibody fragments, to modify an antibody in order to alter (e.g., increase or decrease) its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Heteroconjugate antibodies are also within the scope of the present invention. Heteroconjugate antibodies are composed of two covalently joined antibodies. Such antibodies have, for example, been proposed to target immune cells to unwanted cells (see, e.g., U.S. Pat. No. 4,676,980). It is also contemplated that the heteroconjugate antibodies can be prepared in vitro using known methods in synthetic protein chemistry, including those involving cross-linking agents. For example, immunotoxins can be constructed using a disulfide exchange reaction or by forming a thioether bond. Examples of suitable reagents for this purpose include iminothiolate and methyl-4-mercaptobutyrimidate.

For the purposes of the present invention, it should be appreciated that modified antibodies, or fragments thereof, can comprise any type of variable region that provides for the association of the antibody with human Notch1. In some embodiments, the region is a variable region that may comprise or be derived from any type of mammal that can be induced to mount a humoral response and generate immunoglobulins against the desired antigen. As such, a variable region of modified antibodies can be, for example, of human, murine, non-human primate (e.g. cynomolgus monkeys, macaques, etc.) or rabbit origin. In some embodiments, both a variable and a constant region of a modified immunoglobulin are human. In other embodiments, variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, variable domains in both the heavy and light chains are altered by at least partial replacement of one or more CDRs and, if necessary, by partial framework region replacement and sequence modification and/or alteration. Although the CDRs may be derived from an antibody of the same class or even subclass as the antibody from which the framework regions are derived, it is envisaged that the CDRs may be derived from an antibody of different class and often from an antibody from a different species. It may not be necessary to replace all of the CDRs with all of the CDRs from the donor variable region to transfer the antigen binding capacity of one variable domain to another. Rather, it may only be necessary to transfer those residues that are required to maintain the activity of the antigen-binding site.

Alterations to the variable region notwithstanding, those skilled in the art will appreciate that the modified antibodies of this invention will comprise antibodies (e.g., full-length antibodies or antigen-binding fragments thereof) in which at least a fraction of one or more of the constant region domains has been deleted or otherwise altered so as to provide desired biochemical characteristics, such as increased tumor localization, increased tumor penetration, reduced serum half-life or increased serum half-life when compared with an antibody of approximately the same immunogenicity comprising a native or unaltered constant region. In some embodiments, the constant region of the modified antibodies comprises a human constant region. Modifications to the constant region include additions, deletions, or substitutions of one or more amino acids in one or more domains. The modified antibodies disclosed herein may comprise alterations or modifications to one or more of the three heavy chain constant domains (CH1, CH2 or CH3) and/or to the light chain constant domain (CL). In some embodiments, one or more domains are partially or entirely deleted from the constant regions of the modified antibodies. In some embodiments, the entire CH2 domain has been removed (ΔCH2 constructs). In some embodiments, the omitted constant region domain is replaced by a short amino acid spacer (e.g., 10 aa residues) that provides some of the molecular flexibility typically imparted by the absent constant region.

In certain embodiments, the modified antibodies are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers can, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fc binding and thereby increase tumor localization and/or tumor penetration. Similarly, it may be desirable to simply delete the part of one or more constant region domains that control a specific effector function (e.g., complement Cl q binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or provide for more cytotoxin or carbohydrate attachment.

It is known in the art that the constant region mediates several effector functions. For example, binding of the Cl component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an antibody can bind to a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells, release of inflammatory mediators, placental transfer and control of immunoglobulin production.

In certain embodiments, the anti-Notch1 antibodies provide for altered effector functions that, in turn, affect the biological profile of the administered antibody. For example, in some embodiments, the deletion or inactivation (through point mutations or other means) of a constant region domain may reduce Fc receptor binding of the circulating modified antibody (e.g., anti-Notch1 antibody) thereby increasing tumor localization and/or penetration. In other embodiments, the constant region modifications increase or reduce the serum half-life of the antibody. In some embodiments, the constant region is modified to eliminate disulfide linkages or oligosaccharide moieties allowing for enhanced tumor localization and/or penetration.

In certain embodiments, an anti-Notch1 antibody does not have one or more effector functions. In some embodiments, the antibody has no antibody-dependent cellular cytoxicity (ADCC) activity and/or no complement-dependent cytoxicity (CDC) activity. In certain embodiments, the antibody does not bind to an Fc receptor and/or complement factors. In certain embodiments, the antibody has no effector function.

The present invention further embraces variants and equivalents which are substantially homologous to the chimeric, humanized, and human antibodies, or antibody fragments thereof, set forth herein. These can contain, for example, conservative substitution mutations, i.e. the substitution of one or more amino acids by similar amino acids.

The anti-Notch1 antibodies of the present invention can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blot analysis, radioimmunoassay, ELISA, “sandwich” immunoassay, immunoprecipitation assay, precipitation reaction, gel diffusion precipitin reaction, immunodiffusion assay, agglutination assay, complement-fixation assay, immunoradiometric assay, fluorescent immunoassay, and protein A immunoassay. Such assays are routine and well known in the art (see, e.g., Ausubel et al., Editors, 1994-present, Current Protocols in Molecular Biology, John Wiley & Sons, Inc., New York, N.Y.).

For example, the specific binding of an anti-Notch1 antibody to human Notch1 may be determined using ELISA. An ELISA assay comprises preparing an antigen, coating wells of a 96 well microtiter plate with the antigen, adding to the wells the antibody conjugated to a detectable compound such as an enzymatic substrate (e.g., horseradish peroxidase or alkaline phosphatase), incubating for a period of time and detecting the presence of the binding agent or antibody. In some embodiments, the antibody is not conjugated to a detectable compound, but instead a second conjugated antibody that recognizes the antibody is added to the well. In some embodiments, instead of coating the well with the antigen, the antibody can be coated on the well, antigen is added to the coated well and then a second antibody conjugated to a detectable compound is added. One of skill in the art would be knowledgeable as to the parameters that can be modified and/or optimized to increase the signal detected, as well as other variations of ELISAs that can be used (see, e.g., Ausubel et al., Editors, 1994-present, Current Protocols in Molecular Biology, John Wiley & Sons, Inc., New York, N.Y.).

In another example, the specific binding of an anti-Notch1 antibody to human Notch1 may be determined using FACS. A FACS screening assay may comprise generating a cDNA construct that expresses an antigen as a fusion protein transfecting the construct into cells, expressing the antigen on the surface of the cells, mixing the anti-Notch1 antibody with the transfected cells, and incubating for a period of time. The cells bound by the antibody may be identified by using a secondary antibody conjugated to a detectable compound (e.g., PE-conjugated anti-Fc antibody) and a flow cytometer. One of skill in the art would be knowledgeable as to the parameters that can be modified to optimize the signal detected as well as other variations of FACS that may enhance screening (e.g., screening for blocking antibodies).

The binding affinity of an anti-Notch1 antibody and the on-off rate of an antibody-antigen interaction can be determined by competitive binding assays. In some embodiments, a competitive binding assay is a radioimmunoassay comprising the incubation of labeled antigen (e.g., ³H or ¹²⁵I), or fragment or variant thereof, with the antibody of interest in the presence of increasing amounts of unlabeled antigen, followed by the detection of the antibody bound to the labeled antigen. The affinity of the antibody for the antigen and the on-off rates can be determined from the data by Scatchard plot analysis. In some embodiments, Biacore kinetic analysis is used to determine the binding affinities and on-off rates of antibodies or antibodies that bind Notch1. Biacore kinetic analysis comprises analyzing the binding and dissociation of antibodies from antigens (e.g., Notch proteins) that have been immobilized on the surface of a Biacore chip. In some embodiments, Biacore kinetic analyses are used to determine binding of different antibodies in qualitative epitope competition binding assays.

Thus, the present invention provides methods for generating an antibody that binds the extracellular domain of human Notch1. In some embodiments, the present invention provides methods for generating an antibody that binds the extracellular domain of human Notch1. In some embodiments, the present invention provides methods for generating an antibody that binds a non-ligand binding membrane proximal region of the extracellular domain of human Notch1. In some embodiments, the method for generating an antibody that binds Notch1 comprises using hybridoma techniques. In some embodiments, the method comprises using an extracellular domain of mouse Notch1 or human Notch1 as an immunizing antigen. In some embodiments, the method of generating an antibody that binds Notch1 comprises screening a human phage library. The present invention further provides methods of identifying an antibody that binds human Notch1. In some embodiments, the antibody is identified by screening for binding to Notch1 with flow cytometry (FACS). In some embodiments, the antibody is screened for binding to human Notch1. In some embodiments, the antibody is screened for binding to mouse Notch1. In some embodiments, the antibody is identified by screening for inhibition or blocking of Notch activation.

In certain embodiments, the antibodies described herein are isolated. In certain embodiments, the antibodies described herein are substantially pure.

Non-limiting examples of anti-Notch1 antibodies have been described, for example, in U.S. Pat. No. 8,435,513.

In some embodiments of the present invention, the anti-Notch1 antibodies are polypeptides. The polypeptides can be recombinant polypeptides, natural polypeptides, or synthetic polypeptides that bind the extracellular domain of Notch1. It will be recognized by those of skill in the art that some amino acid sequences of a polypeptide can be varied without significant effect on the structure or function of the protein. Thus, the polypeptides further include variations of the polypeptides which show substantial binding activity to an epitope of the human Notch1 protein. In some embodiments, amino acid sequence variations of polypeptides include deletions, insertions, inversions, repeats, and/or type substitutions.

The polypeptides and variants thereof, can be further modified to contain additional chemical moieties not normally part of the polypeptide. The derivatized moieties can improve the solubility, the biological half-life, or the absorption of the polypeptide. The moieties can also reduce or eliminate any undesirable side effects of the polypeptides and variants. An overview for such chemical moieties can be found in Remington: The Science and Practice of Pharmacy, 22^{st} Edition, 2012, Pharmaceutical Press, London.

The isolated polypeptides described herein can be produced by any suitable method known in the art. Such methods range from direct protein synthesis methods to constructing a DNA sequence encoding isolated polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional variants thereof.

In some embodiments, a DNA sequence encoding a polypeptide of interest may be constructed by chemical synthesis using an oligonucleotide synthesizer. Oligonucleotides can be designed based on the amino acid sequence of the desired polypeptide and by selecting those codons that are favored in the host cell in which the recombinant polypeptide of interest will be produced. Standard methods can be applied to synthesize a polynucleotide sequence encoding a polypeptide of interest. For example, a complete amino acid sequence can be used to construct a back-translated gene. Further, a DNA oligomer containing a nucleotide sequence coding for the particular polypeptide can be synthesized. For example, several small oligonucleotides coding for portions of the desired polypeptide can be synthesized and then ligated. The individual oligonucleotides typically contain 5′ and/or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the polypeptide in a desired host. Proper assembly can be confirmed by nucleotide sequencing, restriction mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding Notch1 antibodies or fragments thereof. For example, recombinant expression vectors can be replicable DNA constructs which have synthetic or cDNA-derived DNA fragments encoding a polypeptide chain of an anti-Notch1 antibody, or fragment thereof, operatively linked to suitable transcriptional or translational regulatory elements derived from mammalian, microbial, viral, or insect genes. A transcriptional unit generally comprises an assembly of (1) a regulatory element or elements having a role in gene expression, for example, transcriptional promoters and/or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can also be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

The choice of an expression vector and control elements depends upon the choice of host. A wide variety of expression host/vector combinations can be employed. Useful expression vectors for eukaryotic hosts include, for example, vectors comprising expression control sequences from SV40, bovine papilloma virus, adenovirus and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Suitable host cells for expression of an anti-Notch1 antibody (or a Notch protein to use as an antigen) include prokaryotes, yeast, insect, or higher eukaryotic cells under the control of appropriate promoters. Prokaryotes include gram-negative or gram-positive organisms, for example, E. coli or Bacilli. Higher eukaryotic cells include established cell lines of mammalian origin as described below. Cell-free translation systems can also be employed.

Various mammalian or insect cell culture systems are used to express recombinant protein. Expression of recombinant proteins in mammalian cells may be preferred because such proteins are generally correctly folded, appropriately modified, and biologically functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived) cell lines, and HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art (see, e.g., Luckow and Summers, 1988, Bio/Technology, 6:47).

The proteins (e.g., antibodies) produced by a transformed host can be purified according to any suitable method. Such methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence and glutathione-S-transferase can be attached to the protein to allow easy purification by passage over an appropriate affinity column. Isolated proteins can be physically characterized using such techniques as proteolysis, high performance liquid chromatography (HPLC), nuclear magnetic resonance (NMR), and x-ray crystallography.

For example, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin is employed, for example, a matrix or substrate having pendant diethylaminoethyl (DEAE) groups. The matrices can be acrylamide, agarose, dextran, cellulose, or other types commonly employed in protein purification. In some embodiments, a cation exchange step is employed. Suitable cation exchangers include various insoluble matrices comprising sulfopropyl or carboxymethyl groups. In some embodiments, a hydroxyapatite media is employed, including but not limited to, ceramic hydroxyapatite (CHT). In some embodiments, one or more reversed-phase HPLC steps employing hydrophobic RP-HPLC media, (e.g., silica gel having pendant methyl or other aliphatic groups), is employed to further purify a protein. Some or all of the foregoing purification steps, in various combinations, can be employed to provide a homogeneous recombinant protein.

In some embodiments, recombinant protein produced in bacterial culture is isolated, for example, by initial extraction from cell pellets, followed by one or more concentration, salting-out, aqueous ion exchange, or size exclusion chromatography steps. In certain embodiments, HPLC is employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Application Pub. Nos. 2008/0312425 and 2009/0187005 and U.S. Pat. No. 7,691,980.

A variety of methods for identifying and producing non-antibody polypeptides that bind with high affinity to a protein target are known in the art. See, e.g., Skerra, 2007, Curr. Opin. Biotechnol., 18:295-304; Hosse et al., 2006, Protein Science, 15:14-27; Gill et al., 2006, Curr. Opin. Biotechnol., 17:653-658; Nygren, 2008, FEBS J., 275:2668-76; and Skerra, 2008, FEBS J., 275:2677-83. In certain embodiments, phage display technology may be used to produce and/or identify a Notch1-binding polypeptide. In certain embodiments, the Notch1-binding polypeptide comprises a protein scaffold of a type selected from the group consisting of protein A, protein G, a lipocalin, a fibronectin domain, an ankyrin consensus repeat domain, and thioredoxin.

In certain embodiments, the anti-Notch1 antibodies can be used in any one of a number of conjugated (e.g., an immunoconjugate or radioconjugate) or non-conjugated forms. In certain embodiments, the antibodies are used in non-conjugated form to harness the subject's natural defense mechanisms including CDC and/or ADCC to eliminate malignant or cancerous cells.

In certain embodiments, the anti-Notch1 antibody is conjugated to a cytotoxic agent. In some embodiments, the cytotoxic agent is a chemotherapeutic agent including, but not limited to, methotrexate, adriamicin, doxorubicin, melphalan, mitomycin C, chlorambucil, daunorubicin or other intercalating agents. In some embodiments, the cytotoxic agent is a enzymatically active toxin of bacterial, fungal, plant, or animal origin, or fragments thereof, including but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, restrictocin, phenomycin, enomycin, and the tricothecenes. In certain embodiments, the cytotoxic agent is a radioactive isotope to produce a radioconjugate or a radioconjugated antibody. A variety of radionuclides are available for the production of radioconjugated antibodies including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷¹Lu, ¹⁸⁶Re, ¹⁸⁸Re and ²¹²Bi. Conjugates of an antibody and one or more small molecule toxins, such as a calicheamicin, maytansinoids, a trichothene, and CC1065, and the derivatives of these toxins that have toxin activity, can also be used. Conjugates of an antibody and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

Heteroconjugate antibodies are also within the scope of the present invention. Heteroconjugate antibodies are composed of two covalently joined antibodies. Such antibodies have, for example, been proposed to target immune cells to unwanted cells (U.S. Pat. No. 4,676,980). It is contemplated that the antibodies can be prepared in vitro using known methods in synthetic protein chemistry, including those involving crosslinking agents.

### IV. POLYNUCLEOTIDES

In certain embodiments, the invention encompasses polynucleotides comprising polynucleotides that encode a polypeptide that specifically binds the extracellular domain of human Notch1 or a fragment of such a polypeptide. The term “polynucleotides that encode a polypeptide” encompasses a polynucleotide which includes only coding sequences for the polypeptide as well as a polynucleotide which includes additional coding and/or non-coding sequences. For example, the invention provides a polynucleotide comprising a nucleic acid sequence that encodes an antibody to human Notch1 or encodes a fragment of such an antibody. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In certain embodiments, the polynucleotide comprises a polynucleotide encoding a polypeptide comprising a sequence selected from the group consisting of SEQ ID NO:3, SEQ ID NO:5, SEQ ID NO:7, SEQ ID NO:9, SEQ ID NO:11, SEQ ID NO:13, SEQ ID NO:21, SEQ ID NO:24, SEQ ID NO:27, SEQ ID NO:30, SEQ ID NO:33, and SEQ ID NO:36. In some embodiments, the polynucleotide comprises a polynucleotide sequence selected from the group consisting of SEQ ID NO:24 and SEQ ID NO:30. In some embodiments, a plasmid comprises a polynucleotide comprising SEQ ID NO:24. In some embodiments, a plasmid comprises a polynucleotide comprising SEQ ID NO:30.

In certain embodiments, the polynucleotide comprises a polynucleotide having a nucleotide sequence at least 80% identical, at least 85% identical, at least 90% identical, at least 95% identical, and in some embodiments, at least 96%, 97%, 98% or 99% identical to a polynucleotide comprising a sequence selected from the group consisting of SEQ ID NO:3, SEQ ID NO:5, SEQ ID NO:7, SEQ ID NO:9, SEQ ID NO:11, SEQ ID NO:13, SEQ ID NO:21, SEQ ID NO:24, SEQ ID NO:27, SEQ ID NO:30, SEQ ID NO:33, and SEQ ID NO:36. Also provided is a polynucleotide that comprises a polynucleotide that hybridizes to SEQ ID NO:3, SEQ ID NO:5, SEQ ID NO:7, SEQ ID NO:9, SEQ ID NO:11, SEQ ID NO:13, SEQ ID NO:21, SEQ ID NO:24, SEQ ID NO:27, SEQ ID NO:30, SEQ ID NO:33, or SEQ ID NO:36. Also provided is a polynucleotide that comprises a polynucleotide that hybridizes to a complement of SEQ ID NO:3, SEQ ID NO:5, SEQ ID NO:7, SEQ ID NO:9, SEQ ID NO:11, SEQ ID NO:13, SEQ ID NO:21, SEQ ID NO:24, SEQ ID NO:27, SEQ ID NO:30, SEQ ID NO:33, and SEQ ID NO:36. In certain embodiments, the hybridization is under conditions of high stringency.

The binding agents of the present invention can be encoded by one or more polynucleotides. For example, in some embodiments, a heavy chain polypeptide is encoded by one polynucleotide and a light chain polypeptide is encoded by a second polynucleotide. In some embodiments, a heavy chain polypeptide and a light chain polypeptide are encoded by one polynucleotide.

In certain embodiments, the polynucleotides comprise the coding sequence for the mature polypeptide fused in the same reading frame to a polynucleotide which aids, for example, in expression and secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to produce the mature form of the polypeptide. The polynucleotides can also encode for a proprotein which is the mature protein plus additional 5′ amino acid residues. A mature protein having a prosequence is a proprotein and is an inactive form of the protein. Once the prosequence is cleaved an active mature protein remains.

In certain embodiments, the polynucleotides comprise the coding sequence for the mature polypeptide fused in the same reading frame to a marker sequence that allows for, for example, purification and/or identification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. In some embodiments, the marker sequence is a FLAG-tag, a peptide of sequence DYKDDDDK (SEQ ID NO:39) which can be used in conjunction with other affinity tags.

The present invention further relates to variants of the hereinabove described polynucleotides encoding, for example, fragments, analogs, and/or derivatives.

In certain embodiments, the present invention provides isolated polynucleotides comprising polynucleotides having a nucleotide sequence at least 80% identical, at least 85% identical, at least 90% identical, at least 95% identical, and in some embodiments, at least 96%, 97%, 98%, or 99% identical to a polynucleotide encoding a polypeptide comprising an antibody, or fragment thereof, described herein.

As used herein, the phrase a polynucleotide having a nucleotide sequence at least, for example, 95% “identical” to a reference nucleotide sequence is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, the polynucleotide variants contain alterations which produce silent substitutions, additions, or deletions, but do not alter the properties or activities of the encoded polypeptide. In some embodiments, the polynucleotide variants contain alterations which do not produce any changes in the amino acid sequence. In some embodiments, polynucleotide variants contain “silent” substitutions due to the degeneracy of the genetic code. Polynucleotide variants can be produced for a variety of reasons, for example, to optimize codon expression for a particular host (e.g., change codons in the human mRNA to those preferred by a bacterial host such as E. coli).

In certain embodiments, the polynucleotides are isolated. In certain embodiments, the polynucleotides are substantially pure.

Vectors and cells comprising the polynucleotides described herein are also provided. In some embodiments, an expression vector comprises a polynucleotide. In some embodiments, a host cell comprises an expression vector comprising the polynucleotide. In some embodiments, a host cell comprises a polynucleotide.

### V. KITS

The present invention provides kits that comprise the antibodies or other agents described herein and that can be used to perform the methods described herein. In some embodiments, a kit comprises an anti-Notch1 antibody in one or more containers. In some embodiments, a kit contains all of the components necessary and/or sufficient to perform a detection assay, for example, detection of a Notch1 mutation, including all controls, directions for performing assays, and any necessary software for analysis and presentation of results. In some embodiments, a kit contains all of the components necessary and/or sufficient to perform a detection assay, for example, detection of Notch1 ICD in a tissue sample, including all controls, directions for performing assays, and any necessary software for analysis and presentation of results. One skilled in the art will readily recognize that the disclosed antibodies or agents of the present invention can be readily incorporated into one of the established kit formats which are well known in the art.

Further provided are kits comprising an anti-Notch1 antibody as well as at least one additional therapeutic agent. In certain embodiments, the additional therapeutic agent is a chemotherapeutic agent. In certain embodiments, the additional therapeutic agent is an angiogenesis inhibitor. In certain embodiments, the additional therapeutic agent is an additional antibody.

Embodiments of the present disclosure can be further defined by reference to the following non-limiting examples, which describe the use of an anti-Notch1 antibody for treatment of cancer. It will be apparent to those skilled in the art that many modifications, both to materials and methods, may be practiced without departing from the scope of the present disclosure.

### EXAMPLES

### Example 1

### Phase 1a Study

An open-label Phase 1a dose escalation study of anti-Notch1 antibody OMP-52M51 in subjects with certain relapsed or refractory solid tumors is on-going. The study includes a dose escalation phase and an expansion phase. The study endpoints include the determination of the safety profile, pharmacokinetics (PK), immunogenicity, pharmacodynamics (PD), and preliminary efficacy.

Prior to enrollment, subjects undergo screening to determine study eligibility. At study entry all subjects undergo assessment for the presence of Notch1 pathway activation using archived tumor tissue or if archival tumor tissue is not available either fresh core or punch needle biopsy tissue.

In the initial phase of the study, dose escalation is performed to determine the maximum tolerated dose of OMP-52M51. The drug is administered intravenously once every 4 weeks (Q4W) at dose levels of 0.25, 0.5, 1.0, and 2.5 and once every three weeks (Q3W) at dose levels of 2.5, 5, and 10 mg/kg. If drug-related toxicity is encountered, drug is administered intravenously once every three weeks at dose levels of 2.0, 1.5, and 1.0 mg/kg. No dose escalation or dose reduction is allowed within a dose cohort.

Starting at the initial dose level of 0.25 mg/kg, an accelerated titration approach is taken such that in each cohort, a minimum of one subject per dosing cohort is enrolled and dose escalation up to the next (higher) dose level is allowed provided that no grade 2 study drug-related toxicities or dose-limiting toxicities (DLTs) are observed. Additional subjects are enrolled in the cohort provided that they are identified and receive the first dose within 2 weeks of the first subject. Prior to enrollment of the first subject at each subsequent dose level, all subject(s) in the previous cohort must be observed for a minimum of 28 days (21 days if using a Q3W dosing schedule).

If two subjects experience a grade 2 or greater study drug-related toxicity or any subject experiences a DLT, then accelerated titration will be terminated and a minimum of 3 subjects will be enrolled in the current dosing cohort and any subsequent cohorts. If a DLT is observed then at least 6 subjects will be enrolled in the dosing cohort, unless a second DLT occurs prior to enrollment of all 6 subjects. No two subjects will initiate dosing on the same day with a dose that has not been previously tested. However, additional subjects may be enrolled after 24 hours has elapsed. If two subjects experience a DLT, no further subjects will be dosed at that level and 3 additional subjects will be added to either the preceding dose cohort (unless 6 subjects have already been treated at that dose level) or an intermediate dose level. Subjects will be assessed for DLTs from Days 0-28 for Q4W dosing and Days 0-21 for Q3W dosing.

A summary of the study as of Feb. 12, 2014 is found in Table 2A. A summary of the study as of Feb. 24, 2015 is found in Table 2B.

TABLE 2A

| | Dose mg/kg | | | | |
|---|---|---|---|---|---|
| | Q4W | | | | Q3W |
| | 0.25 | 0.5 | 1 | 2.5 | 2.5 |
| No. subjects | 1 | 3 | 3 | 6 | 7 |
| treated | | | | | |
| No. subjects | 1 | 3 | 2 | 6 | 5 |
| evaluable for | | | | | |
| DLT | | | | | |
| No. of | 0 | 0 | 0 | 0 | 4 |
| subjects on | | | | | |
| treatment | | | | | |
| No. of DLTs | 0 | 0 | 0 | 1 | 1 |
| Diarrhea | 1 | 2 | 2 | 4 | 5 |

TABLE 2B

| | Dose mg/kg | | | | | | | |
|---|---|---|---|---|---|---|---|---|
| | Q4W | | | | Q3W | | | |
| | 0.25 | 0.5 | 1 | 2.5 | 2.5 | 2.0 | 1.0 | 1.5 |
| No. subjects | 1 | 3 | 3 | 6 | 7 | 4 | 3 | 6 |
| treated | | | | | | | | |
| No. subjects | 1 | 3 | 2 | 6 | 6 | 4 | 3 | 6 |
| evaluable for | | | | | | | | |
| DLT | | | | | | | | |
| No. of | 0 | 0 | 0 | 0 | 0 | 0 | 0 | 3 |
| subjects on | | | | | | | | |
| treatment | | | | | | | | |
| No. of DLTs | 0 | 0 | 0 | 1 | 1 | 1 | 0 | 0 |
| Diarrhea | 1 | 2 | 2 | 5 | 6 | 4 | 2 | 2 |

In the dose escalation phase of the study, 7 patients had Notch1 ICD high tumors, 19 had Notch1 ICD low tumors, and Notch1 ICD tumor status was not determined for 7 patients due to lack of tumor material.

Further, the dose expansion cohort phase of the study has begun to enroll patients. This cohort enrolls only patients with Notch1 ICD high tumors.

### Example 2

### Subject with Adenoid Cystic Carcinoma

One subject enrolled in the 2.5 mg/kg Q3W cohort, is a 28-year-old male with recurrent adenoid cystic carcinoma with metastases in the liver, lungs, and bone. The subject's tumor has a Notch1 mutation in exon 34 of the Notch1 gene that is predicted to cause a frameshift mutation with early termination in translation. The standardized nomenclature for this mutation is NM_017617.3(NOTCH1):c.7398_7401del p.52467f5*. This mutation is in the PEST domain of Notch1 and is believed to be an activating mutation. From March 2013 to December 2013 the subject had undergone four prior lines of treatment. These treatments included cisplatin/adriamycin/cytoxan, carboplatin/vinorelbine, ISIS 481464, and cetuximab and the subject was noted to have progression of disease after each of these treatments.

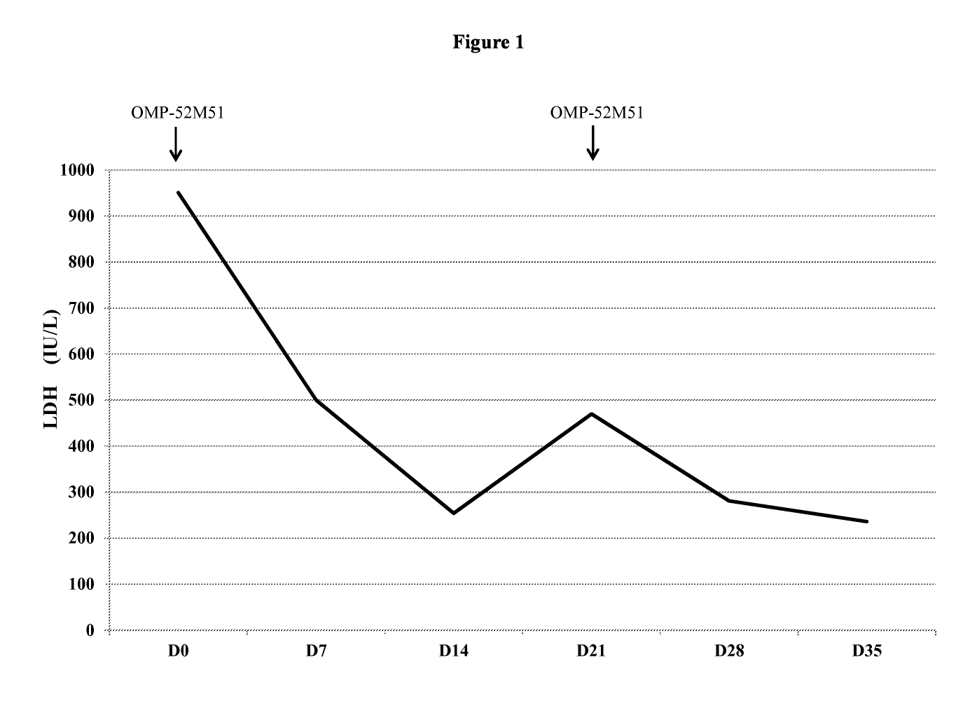

Many cancer types can raise lactate dehydrogenase (LDH) levels, and measuring LDH levels can be helpful in monitoring treatment when they are elevated. Since the subject's LDH level was elevated, this marker was used to monitor treatment with OMP-52M51. At Day 0, the subject's LDH level was 1125 IU/L. On Day 7 after the first dose of OMP-52M51 the subject's LDH level had decreased to 500 IU/L and on Day 14 the DLH level had further decreased to 254 IU/L. On Day 21 the subject's LDH level had risen to ˜475 IU/L, however after a second dose of OMP-52M51, the LDH level decreased again to ˜280 IU/L on Day 28, with an additional small decrease on Day 35 (see FIG. 1).

The subject had a significant amount of pain from bone metastases. Prior to treatment with OMP-52M51, the subject was taking 60 mg/day of oxycontin. After the first dose of OMP-52M51, from Days 2-20, the subject experienced a notable decrease in bone pain and was able to decrease his oxycontin intake to 0-8 mg/day. On Day 21, the subject was experiencing increased pain, however after a second dose of OMP-52M51, the subject reported a reduction in pain again at Days 23 and 28.

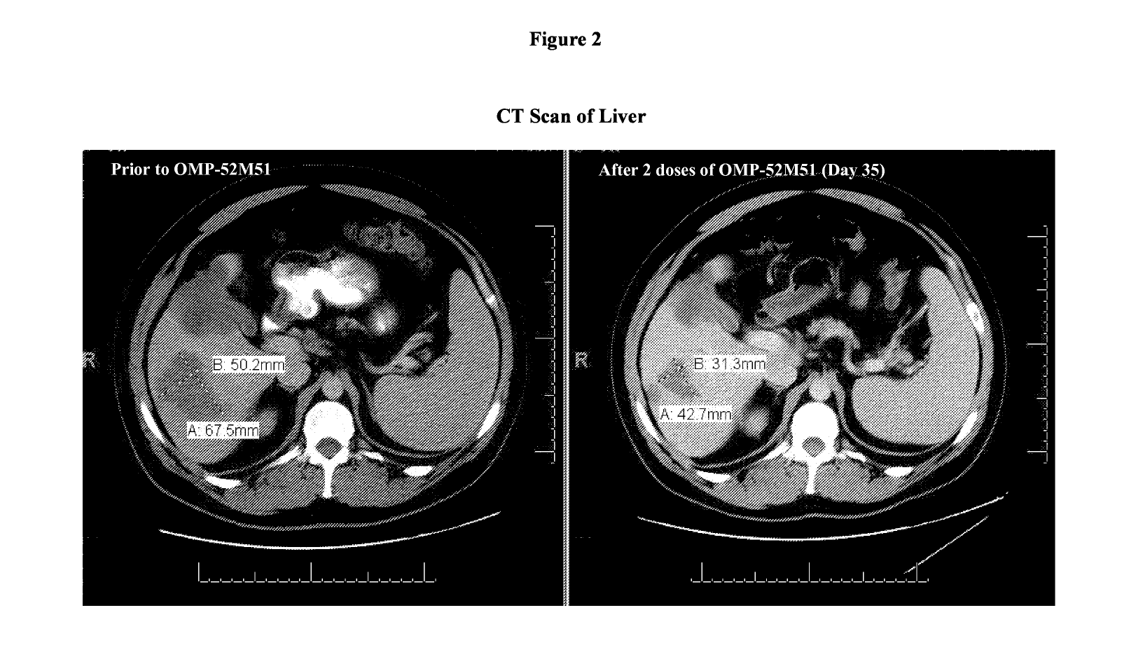

CT scans were obtained of the subject's chest, abdomen and pelvis area after two doses of OMP-52M51 and compared to CT scans done approximately 5 weeks earlier. The nodules in the lungs remained stable and there appeared to be no new metastatic disease. CT scans of the liver showed a significant decrease in size of several liver metastatic masses (FIG. 2). In addition, a reduction in the number of liver metastases and sclerosing bone metastases was reported.

Thus, treatment with the anti-Notch1 antibody OMP-52M51 appears to have an efficacious affect on an adenoid cystic carcinoma containing an activating Notch1 mutation. These early results are surprising and unexpected given the fact that the subject's tumor was shown to be refractory to several different treatments, including chemotherapy, antisense therapy, and a monoclonal antibody targeted to a cellular kinase.

### Example 3

### Notch1 Signaling Assay

As described above in Example 2, a Notch1 mutation, c.7398_7401del,p.S2467fs, was identified in the tumor of the ACC patient. To determine the effect of this mutation on Notch1 signaling, a plasmid that expressed the full-length mutant Notch1.S2467fs protein was constructed by using a synthesized DNA fragment with the desired 4-bp deletion (c.7398_7401del). Controls included, an empty vector, Notch1 wild-type (Notch1.WT), Notch1 with mutation in EGF9 domain (Notch1.F357delF), and Notch1 with activating mutation (Notch1.G2427fs) which had been previously identified in a breast cancer patient-derived xenograft tumor (OMP-B40).

Human PC3 cells were transfected with the test plasmids and a firefly luciferase reporter vector (8×CBF-luciferase reporter) that is responsive to Notch signaling. The cells were also transfected with a Renilla luciferase reporter (Promega, Madison Wis.) as an internal control for transfection efficiency. Transfected PC3 cells were incubated overnight at 37 C. Luciferase activity was determined using the Dual-Glo Luciferase Assay System (Promega, Madison Wis.) and with firefly luciferase activity normalized to Renilla luciferase activity.

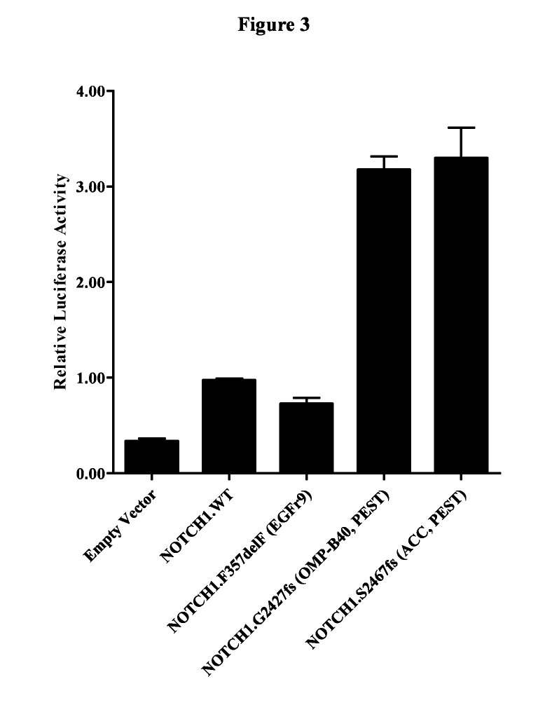

The luciferase activity for each of the Notch1 proteins is relative to the activity of wild-type Notch1 (NOTCH1-WT) which is set at 1.0. As shown in FIG. 3, Notch1.S2467fs demonstrated 3.3-fold higher activity than wild-type Notch1 (NOTCH1.WT) in the absence of ligands. This data indicates that this frameshift mutation (Notch1.S2467fs) in the PEST domain of Notch1 is constitutively active. The degree of constitutive activation was similar to that observed for a different frameshift mutation (Notch1.G2427fs) previously identified in a breast patient-derived xenograft tumor (OMP-B40).

### Example 4

### Notch1 ICD Expression Assessed by IHC

A Notch1 ICD immunohistochemistry (IHC) assay was developed and optimized using the rabbit monoclonal antibody D3B8 (#4147, Cell signaling Technology, Danvers, Mass.). This antibody detects the Notch1 intracellular domain (ICD) only when the polypeptide is released by cleavage between Gly1753 and Val1754. The antibody does not recognize full-length Notch1 or Notch1 fragments cleaved at other positions. 4 μm-thick FFPE sections were cut and mounted on coated glass slides. Tissues were stained on a Ventana BenchMark ULTRA instrument using Ventana reagents (Ventana Medical Systems, Inc. Tucson Ariz.). Sections were treated with extended Cell Conditioning 1 followed by antibody incubation for 30 minutes at 37° C., dispensed from a userdefined prep-kit with antibody at 9 μg/ml. Antibody was detected using ultraView and Amplification Kits with diaminobenzidine (DAB) and counterstaining with hematoxylin.

The slides were analyzed using an Aperio instrument (Leica Biosystems). The staining intensity of each tumor nucleus (0: no expression, 1: weak expression, 2: moderate expression, 3: strong expression) was measured and nuclei of each staining level were counted and a percentage for each type was calculated. The data was combined into a weighted H-score for each tissue section: H-score=[3×(% 3+nuclei)]+[2×(% 2+nuclei)]+[1×(% 1+nuclei)]. Positive and negative controls included human tissue sections purchased from Folio Biosciences (Columbus Ohio) as well as patient-derived xenograft (PDX) samples from the OncoMed tumor bank with known expression levels for Notch1 ICD.

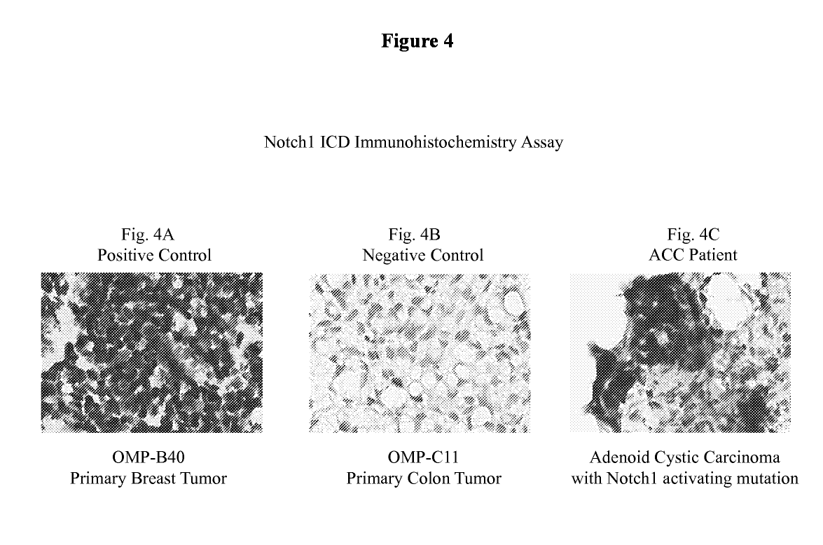

Using this IHC assay, tumor samples from the patient described in Example 2 were examined. As shown in FIG. 4, the patient's tumor had a high level of Notch1 ICD, in agreement with the observation that the patient's tumor had an activating Notch1 mutation. In addition, another patient treated with OMP-52M51 who had stable disease for up to ten months was shown to have a tumor expressing high levels of Notch1 ICD. In contrast, a patient whose disease progressed during treatment with OMP-52M51 was shown to have a tumor with low or undetectable levels of Notch1 ICD. These results provide a very strong basis for a Notch1 ICD IHC-based predictive assay for identifying tumors and patients likely to respond to treatment with the anti-Notch1 therapeutic antibody OMP-52M51.

It is understood that the examples and embodiments described herein are for illustrative purposes only and that various modifications or changes in light thereof will be suggested to persons skilled in the art and are to be included within the spirit and purview of this application.

All publications, patents, and patent applications cited herein are hereby incorporated by reference in their entirety for all purposes to the same extent as if each individual publication, patent or patent application were specifically and individually indicated to be so incorporated by reference.

Following are the sequences disclosed in the application:

Human Notch1 polynucleotide encoding amino acids 1427-1732
(SEQ ID NO: 1)
CACATCCTGGACTACAGCTTCGGGGGTGGGGCCGGGCGCGACATCCCCCCGCCGCTGATC
GAGGAGGCGTGCGAGCTGCCCGAGTGCCAGGAGGACGCGGGCAACAAGGTCTGCAGCCTG
CAGTGCAACAACCACGCGTGCGGCTGGGACGGCGGTGACTGCTCCCTCAACTTCAATGAC
CCCTGGAAGAACTGCACGCAGTCTCTGCAGTGCTGGAAGTACTTCAGTGACGGCCACTGT
GACAGCCAGTGCAACTCAGCCGGCTGCCTCTTCGACGGCTTTGACTGCCAGCGTGCGGAA
GGCCAGTGCAACCCCCTGTACGACCAGTACTGCAAGGACCACTTCAGCGACGGGCACTGC
GACCAGGGCTGCAACAGCGCGGAGTGCGAGTGGGACGGGCTGGACTGTGCGGAGCATGTA
CCCGAGAGGCTGGCGGCCGGCACGCTGGTGGTGGTGGTGCTGATGCCGCCGGAGCAGCTG
CGCAACAGCTCCTTCCACTTCCTGCGGGAGCTCAGCCGCGTGCTGCACACCAACGTGGTC
TTCAAGCGTGACGCACACGGCCAGCAGATGATCTTCCCCTACTACGGCCGCGAGGAGGAG
CTGCGCAAGCACCCCATCAAGCGTGCCGCCGAGGGCTGGGCCGCACCTGACGCCCTGCTG
GGCCAGGTGAAGGCCTCGCTGCTCCCTGGTGGCAGCGAGGGTGGGCGGCGGCGGAGGGAG
CTGGACCCCATGGACGTCCGCGGCTCCATCGTCTACCTGGAGATTGACAACCGGCAGTGT
GTGCAGGCCTCCTCGCAGTGCTTCCAGAGTGCCACCGACGTGGCCGCATTCCTGGGAGCG
CTCGCCTCGCTGGGCAGCCTCAACATCCCCTACAAGATCGAGGCCGTGCAGAGTGAGACC
GTGGAGCCGCCCCCGCCG
Human Notch1 amino acids 1427-1732
(SEQ ID NO: 2)
HILDYSFGGGAGRDIPPPLIEEACELPECQEDAGNKVCSLQCNNHACGWDGGDCSLNFND
PWKNCTQSLQCWKYFSDGHCDSQCNSAGCLFDGFDCQRAEGQCNPLYDQYCKDHFSDGHC
DQGCNSAECEWDGLDCAEHVPERLAAGTLVVVVLMPPEQLRNSSFHFLRELSRVLHTNVV
FKRDAHGQQMIFPYYGREEELRKHPIKRAAEGWAAPDALLGQVKASLLPGGSEGGRRRRE
LDPMDVRGSIVYLEIDNRQCVQASSQCFQSATDVAAFLGALASLGSLNIPYKIEAVQSET
VEPPPP
Mouse antibody 52M51 sequences
52M51 Heavy chain polynucleotide sequence
(SEQ ID NO: 3)
ATGGAATGGACCTGGGTCTTTCTCTTCCTCCTGTCAGTAACTGCAGGTGTCCACTCCCAG
GTTCAGCTGCAGCAGTCTGGAGCTGAGCTGATGAAGCCTGGGGCCTCAGTGAAGATATCC
TGCAAGGCTGCTGGCTACACAATGAGAGGCTACTGGATAGAGTGGATAAAGCAGAGGCCT
GGACATGGCCTTGAGTGGATTGGACAGATTTTACCTGGAACTGGGAGAACTAACTACAAT
GAGAAGTTCAAGGGCAAGGCCACATTCACTGCAGATACATCCTCCAACACAGCCAACATG
CAACTCAGCAGCCTGACATCTGAGGACTCTGCCGTCTATTACTGTGCAAGATTTGATGGT
AACTACGGTTACTATGCTATGGACTACTGGGGTCAAGGATCCTCAGTCACCGTCTCCTCA
GCCAAAACGACACCCCCATCTGTCTATCCACTGGCCCCTGGATCTGCTGCCCAAACTAAC
TCCATGGTGACCCTGGGATGCCTGGTCAAGGGCTATTTCCCTGAGCCAGTGACAGTGACC
TGGAACTCTGGATCCCTGTCCAGCGGTGTGCACACCTTCCCAGCTGTCCTGCAGTCTGAC
CTCTACACTCTGAGCAGCTCAGTGACTGTCCCCTCCAGCCCTCGGCCCAGCGAGACCGTC
ACCTGCAACGTTGCCCACCCGGCCAGCAGCACCAAGGTGGACAAGAAAATTGTGCCCAGG
GATTGTGGTTGTAAGCCTTGCATATGTACAGTCCCAGAAGTATCATCTGTCTTCATCTTC
CCCCCAAAGCCCAAGGATGTCCTCACCATTACTCTGACTCCTAAGGTCACGTGTGTTGTG
GTAGACATCAGCAAGGATGATCCCGAGGTCCAGTTCAGCTGGTTTGTAGATGATGTGGAG
GTGCACACAGCTCAGACGCAACCCCGGGAGGAGCAGTTCAACAGCACTTTCCGCTCAGTC
AGTGAACTTCCCATCATGCACCAGGACTGGCTCAATGGCAAGGAGTTCAAATGCAGGGTC
AACAGTGCAGCTTTCCCTGCCCCCATCGAGAAAACCATATCCAAAACCAAAGGCAGACCG
AAGGCTCCACAGGTGTACACCATTCCACCTCCCAAGGAGCAGATGGCCAAGGATAAAGTC
AGTCTGACCTGCATGATAACAGACTTCTTCCCTGAAGACATAACAGTGGAGTGGCAGTGG
AATGGGCAGCCAGCGGAGAACTACAAGAACACTCAGCCCATCATGAACACGAATGGCTCT
TACTTCGTCTACAGCAAGCTCAATGTGCAGAAGAGCAACTGGGAGGCAGGAAATACTTTC
ACCTGCTCTGTGTTACATGAGGGCCTGCACAACCACCATACTGAGAAGAGCCTCTCCCAC
TCTCCTGGTAAATGA
52M51 Heavy chain amino acid sequence-predicted signal sequence
is underlined
(SEQ ID NO: 4)
MEWTWVFLFLLSVTAGVHSQVQLQQSGAELMKPGASVKISCKAAGYTMRGYWIEWIKQRP
GHGLEWIGQILPGTGRTNYNEKFKGKATFTADTSSNTANMQLSSLTSEDSAVYYCARFDG
NYGYYAMDYWGQGSSVTVSSAKTTPPSVYPLAPGSAAQTNSMVTLGCLVKGYFPEPVTVT
WNSGSLSSGVHTFPAVLQSDLYTLSSSVTVPSSPRPSETVTCNVAHPASSTKVDKKIVPR
DCGCKPCICTVPEVSSVFIFPPKPKDVLTITLTPKVTCVVVDISKDDPEVQFSWFVDDVE
VHTAQTQPREEQFNSTFRSVSELPIMHQDWLNGKEFKCRVNSAAFPAPIEKTISKTKGRP
KAPQVYTIPPPKEQMAKDKVSLTCMITDFFPEDITVEWQWNGQPAENYKNTQPIMNTNGS
YFVYSKLNVQKSNWEAGNTFTCSVLHEGLHNHHTEKSLSHSPGK
52M51 Heavy chain variable region polynucleotide sequence
(SEQ ID NO: 5)
ATGGAATGGACCTGGGTCTTTCTCTTCCTCCTGTCAGTAACTGCAGGTGTCCACTCCCAG
GTTCAGCTGCAGCAGTCTGGAGCTGAGCTGATGAAGCCTGGGGCCTCAGTGAAGATATCC
TGCAAGGCTGCTGGCTACACAATGAGAGGCTACTGGATAGAGTGGATAAAGCAGAGGCCT
GGACATGGCCTTGAGTGGATTGGACAGATTTTACCTGGAACTGGGAGAACTAACTACAAT
GAGAAGTTCAAGGGCAAGGCCACATTCACTGCAGATACATCCTCCAACACAGCCAACATG
CAACTCAGCAGCCTGACATCTGAGGACTCTGCCGTCTATTACTGTGCAAGATTTGATGGT
AACTACGGTTACTATGCTATGGACTACTGGGGTCAAGGATCCTCAGTCACCGTCTCCTCA
52M51 Heavy chain variable region amino acid sequence-predicted
signal sequence is underlined
(SEQ ID NO: 6)
MEWTWVFLFLLSVTAGVHSQVQLQQSGAELMKPGASVKISCKAAGYTMRGYWIEWIKQRP
GHGLEWIGQILPGTGRTNYNEKFKGKATFTADTSSNTANMQLSSLTSEDSAVYYCARFDG
NYGYYAMDYWGQGSSVTVSSAKTTPPSVYPLAPGSAAQTNSMVTLGCLVKGYFPEPVTVT
52M51 Heavy chain variable region polynucleotide sequence
without predicted signal sequence
(SEQ ID NO: 7)
CAGGTTCAGCTGCAGCAGTCTGGAGCTGAGCTGATGAAGCCTGGGGCCTCAGTGAAGATA
TCCTGCAAGGCTGCTGGCTACACAATGAGAGGCTACTGGATAGAGTGGATAAAGCAGAGG
CCTGGACATGGCCTTGAGTGGATTGGACAGATTTTACCTGGAACTGGGAGAACTAACTAC
AATGAGAAGTTCAAGGGCAAGGCCACATTCACTGCAGATACATCCTCCAACACAGCCAAC
ATGCAACTCAGCAGCCTGACATCTGAGGACTCTGCCGTCTATTACTGTGCAAGATTTGAT
GGTAACTACGGTTACTATGCTATGGACTACTGGGGTCAAGGATCCTCAGTCACCGTCTCC
TCA
52M51 Heavy chain variable region amino acid sequence without
predicted signal sequence
(SEQ ID NO: 8)
QVQLQQSGAELMKPGASVKISCKAAGYTMRGYWIEWIKQRPGHGLEWIGQILPGTGRTNY
NEKFKGKATFTADTSSNTANMQLSSLTSEDSAVYYCARFDGNYGYYAMDYWGQGSSVTVS
SA
52M51 Light chain polynucleotide sequence
(SEQ ID NO: 9)
ATGGCCTGGATTTCACTTATACTCTCTCTCCTGGCTCTCAGCTCAGGGGCCATTTCCCAG
GCTGTTGTGACTCAGGAATCTGCACTCACCACATCACCTGGTGAAACAGTCACACTCACT
TGTCGCTCAAGTACTGGGGCTGTTACAACTAGTAACTACGCCAACTGGGTCCAAGAAAAA
CCTGATCATTTATTCACTGGTCTAATAGGTGGTACCAACAACCGAGCTCCAGGTGTTCCT
GCCAGATTCTCAGGCTCCCTGATTGGAGACAAGGCTGCCCTCACCATCACAGGGGCACAG
ACTGAGGATGAGGCAATATATTTCTGTGCTCTATGGTACAGCAACCACTGGGTGTTCGGT
GGAGGAACCAAACTGACTGTCCTAGGCCAGCCCAAGTCTTCGCCATCAGTCACCCTGTTT
CCACCTTCCTCTGAAGAGCTCGAGACTAACAAGGCCACACTGGTGTGTACGATCACTGAT
TTCTACCCAGGTGTGGTGACAGTGGACTGGAAGGTAGATGGTACCCCTGTCACTCAGGGT
ATGGAGACAACCCAGCCTTCCAAACAGAGCAACAACAAGTACATGGCTAGCAGCTACCTG
ACCCTGACAGCAAGAGCATGGGAAAGGCATAGCAGTTACAGCTGCCAGGTCACTCATGAA
GGTCACACTGTGGAGAAGAGTTTGTCCCGTGCTGACTGTTCCTAG
52M51 Light chain amino acid sequence-predicted signal
sequence is underlined
(SEQ ID NO: 10)
MAWISLILSLLALSSGAISQAVVTQESALTTSPGETVTLTCRSSTGAVTTSNYANWVQEK
PDHLFTGLIGGTNNRAPGVPARFSGSLIGDKAALTITGAQTEDEAIYFCALWYSNHWVFG
GGTKLTVLGQPKSSPSVTLFPPSSEELETNKATLVCTITDFYPGVVTVDWKVDGTPVTQG
METTQPSKQSNNKYMASSYLTLTARAWERHSSYSCQVTHEGHTVEKSLSRADCS
52M51 Light chain variable region polynucleotide sequence
(SEQ ID NO: 11)
ATGGCCTGGATTTCACTTATACTCTCTCTCCTGGCTCTCAGCTCAGGGGCCATTTCCCAG
GCTGTTGTGACTCAGGAATCTGCACTCACCACATCACCTGGTGAAACAGTCACACTCACT
TGTCGCTCAAGTACTGGGGCTGTTACAACTAGTAACTACGCCAACTGGGTCCAAGAAAAA
CCTGATCATTTATTCACTGGTCTAATAGGTGGTACCAACAACCGAGCTCCAGGTGTTCCT
GCCAGATTCTCAGGCTCCCTGATTGGAGACAAGGCTGCCCTCACCATCACAGGGGCACAG
ACTGAGGATGAGGCAATATATTTCTGTGCTCTATGGTACAGCAACCACTGGGTGTTCGGT
GGAGGAACCAAACTGACTGTCCTAGGC
52M51 Light chain variable region amino acid sequence-predicted
signal sequence is underlined
(SEQ ID NO: 12)
MAWISLILSLLALSSGAISQAVVTQESALTTSPGETVTLTCRSSTGAVTTSNYANWVQEK
PDHLFTGLIGGTNNRAPGVPARFSGSLIGDKAALTITGAQTEDEAIYFCALWYSNHWVFG
GGTKLTVLGQPKSSPSVTLFPPSSEELETNKATLVCTITDFYPGVVTVDWKVDGTPVTQG
52M51 Light chain variable region polynucleotide sequence
without predicted signal sequence
(SEQ ID NO: 13)
CAGGCTGTTGTGACTCAGGAATCTGCACTCACCACATCACCTGGTGAAACAGTCACACTC
ACTTGTCGCTCAAGTACTGGGGCTGTTACAACTAGTAACTACGCCAACTGGGTCCAAGAA
AAACCTGATCATTTATTCACTGGTCTAATAGGTGGTACCAACAACCGAGCTCCAGGTGTT
CCTGCCAGATTCTCAGGCTCCCTGATTGGAGACAAGGCTGCCCTCACCATCACAGGGGCA
CAGACTGAGGATGAGGCAATATATTTCTGTGCTCTATGGTACAGCAACCACTGGGTGTTC
GGTGGAGGAACCAAACTGACTGTCCTAGGC
52M51 Light chain variable region amino acid sequence without
predicted signal sequence
(SEQ ID NO: 14)
QAVVTQESALTTSPGETVTLTCRSSTGAVTTSNYANWVQEKPDHLFTGLIGGTNNRAPGV
PARFSGSLIGDKAALTITGAQTEDEAIYFCALWYSNHWVFGGGTKLTVLG
52M51 Heavy chain CDR1
(SEQ ID NO: 15)
RGYWIE
52M51 Heavy chain CDR2
(SEQ ID NO: 16)
QILPGTGRTNYNEKFKG
52M51 Heavy chain CDR3
(SEQ ID NO: 17)
FDGNYGYYAMDY
52M51 Light chain CDR1
(SEQ ID NO: 18)
RSSTGAVTTSNYAN
52M51 Light chain CDR2
(SEQ ID NO: 19)
GTNNRAP
52M51 Light chain CDR3
(SEQ ID NO: 20)
ALWYSNHWVFGGGTKL
Humanized 52M51 sequences
52M51-H4 Heavy chain polynucleotide sequence
(SEQ ID NO: 21)
ATGGATTGGACATGGAGGGTGTTCTGCCTCCTCGCTGTGGCTCCTGGAGTCCTGAGCCAG
GTCCAGCTCGTCCAGAGCGGGGCTGAAGTCAAGAAGCCTGGCGCTAGCGTCAAAATCAGC
TGTAAGGTCAGCGGATACACACTGAGGGGATACTGGATCGAGTGGGTGAGGCAGGCTCCA
GGAAAGGGCCTGGAATGGATCGGCCAGATCCTGCCTGGAACCGGAAGGACAAATTACAAT
GAGAAGTTTAAGGGAAGGGTCACAATGACAGCAGACACAAGCACAGACACAGCTTATATG
GAACTCAGCTCCCTCAGATCCGAGGACACCGCTGTCTACTATTGTGCCAGGTTCGATGGA
AATTACGGATACTATGCCATGGATTACTGGGGACAGGGGACAACGGTCACCGTGAGCTCA
GCCAGCACAAAGGGCCCTAGCGTCTTCCCTCTGGCTCCCTGCAGCAGGAGCACCAGCGAG
AGCACAGCCGCCCTGGGCTGCCTGGTCAAGGACTACTTCCCCGAACCGGTGACGGTGTCG
TGGAACTCAGGCGCTCTGACCAGCGGCGTGCACACCTTCCCAGCTGTCCTACAGTCCTCA
GGACTCTACTCCCTCAGCAGCGTGGTGACCGTGCCCTCCAGCAACTTCGGCACCCAGACC
TACACCTGCAACGTAGATCACAAGCCCAGCAACACCAAGGTGGACAAGACAGTTGAGCGC
AAATGTTGTGTCGAGTGCCCACCGTGCCCAGCACCACCTGTGGCAGGACCGTCAGTCTTC
CTCTTCCCCCCAAAACCCAAGGACACCCTCATGATCTCCCGGACCCCTGAGGTCACGTGC
GTGGTGGTGGACGTGAGCCACGAAGACCCCGAGGTCCAGTTCAACTGGTACGTGGACGGC
GTGGAGGTGCATAATGCCAAGACAAAGCCACGGGAGGAGCAGTTCAACAGCACGTTCCGT
GTGGTCAGCGTCCTCACCGTTGTGCACCAGGACTGGCTGAACGGCAAGGAGTACAAGTGC
AAGGTCTCCAACAAAGGCCTCCCAGCCCCCATCGAGAAAACCATCTCCAAAACCAAAGGG
CAGCCCCGAGAACCACAGGTGTACACCCTGCCCCCATCCCGGGAGGAGATGACCAAGAAC
CAGGTCAGCCTGACCTGCCTGGTCAAAGGCTTCTACCCCAGCGACATCGCCGTGGAGTGG
GAGAGCAATGGGCAGCCGGAGAACAACTACAAGACCACACCTCCCATGCTGGACTCCGAC
GGCTCCTTCTTCCTCTACAGCAAGCTCACCGTGGACAAGAGCAGGTGGCAGCAGGGGAAC
GTCTTCTCATGCTCCGTGATGCATGAGGCTCTGCACAACCACTACACGCAGAAGAGCCTC
TCCCTGTCTCCGGGTAAATGA
52M5 1-H4 Heavy chain amino acid sequence-predicted signal
sequence underlined
(SEQ ID NO: 22)
MDWTWRVFCLLAVAPGVLSQVQLVQSGAEVKKPGASVKISCKVSGYTLRGYWIEWVRQAP
GKGLEWIGQILPGTGRTNYNEKFKGRVTMTADTSTDTAYMELSSLRSEDTAVYYCARFDG
NYGYYAMDYWGQGTTVTVSSASTKGPSVFPLAPCSRSTSESTAALGCLVKDYFPEPVTVS
WNSGALTSGVHTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDPEVQFNWYVDG
VEVHNAKTKPREEQFNSTFRVVSVLTVVHQDWLNGKEYKCKVSNKGLPAPIEKTISKTKG
QPREPQVYTLPPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK
52M51-H4 Heavy chain amino acid sequence without predicted
signal sequence
(SEQ ID NO: 23)
QVQLVQSGAEVKKPGASVKISCKVSGYTLRGYWIEWVRQAPGKGLEWIGQILPGTGRTNY
NEKFKGRVTMTADTSTDTAYMELSSLRSEDTAVYYCARFDGNYGYYAMDYWGQGTTVTVS
SASTKGPSVFPLAPCSRSTSESTAALGCLVKDYFPEPVTVSWNSGALTSGVHTFPAVLQS
SGLYSLSSVVTVPSSNFGTQTYTCNVDHKPSNTKVDKTVERKCCVECPPCPAPPVAGPSV
FLFPPKPKDTLMISRTPEVTCVVVDVSHEDPEVQFNWYVDGVEVHNAKTKPREEQFNSTF
RVVSVLTVVHQDWLNGKEYKCKVSNKGLPAPIEKTISKTKGQPREPQVYTLPPSREEMTK
NQVSLTCLVKGFYPSDIAVEWESNGQPENNYKTTPPMLDSDGSFFLYSKLTVDKSRWQQG
NVFSCSVMHEALHNHYTQKSLSLSPGK
52M51-H4 Heavy chain variable region polynucleotide sequence
(SEQ ID NO: 24)
ATGGATTGGACATGGAGGGTGTTCTGCCTCCTCGCTGTGGCTCCTGGAGTCCTGAGCCAG
GTCCAGCTCGTCCAGAGCGGGGCTGAAGTCAAGAAGCCTGGCGCTAGCGTCAAAATCAGC
TGTAAGGTCAGCGGATACACACTGAGGGGATACTGGATCGAGTGGGTGAGGCAGGCTCCA
GGAAAGGGCCTGGAATGGATCGGCCAGATCCTGCCTGGAACCGGAAGGACAAATTACAAT
GAGAAGTTTAAGGGAAGGGTCACAATGACAGCAGACACAAGCACAGACACAGCTTATATG
GAACTCAGCTCCCTCAGATCCGAGGACACCGCTGTCTACTATTGTGCCAGGTTCGATGGA
AATTACGGATACTATGCCATGGATTACTGGGGACAGGGGACAACGGTCACCGTGAGCTCA
GCC
52M51-H4 Heavy chain variable region amino acid sequence-
predicted signal sequence underlined
(SEQ ID NO: 25)
MDWTWRVFCLLAVAPGVLSQVQLVQSGAEVKKPGASVKISCKVSGYTLRGYWIEWVRQAP
GKGLEWIGQILPGTGRTNYNEKFKGRVTMTADTSTDTAYMELSSLRSEDTAVYYCARFDG
NYGYYAMDYWGQGTTVTVSSA
52M51-H4 Heavy chain variable region amino acid sequence without
predicted signal sequence
(SEQ ID NO: 26)
QVQLVQSGAEVKKPGASVKISCKVSGYTLRGYWIEWVRQAPGKGLEWIGQILPGTGRTNY
NEKFKGRVTMTADTSTDTAYMELSSLRSEDTAVYYCARFDGNYGYYAMDYWGQGTTVTVS
SA
52M51-L3 Light chain polynucleotide sequence
(SEQ ID NO: 27)
ATGAGCGTCCCTACAATGGCTTGGATGATGCTCCTGCTGGGACTCCTGGCTTATGGAAGC
GGAGTGGATAGCCAGGCCGTCGTCACACAGGAACCTAGCCTCACCGTTAGCCCTGGAGGA
ACAGTCACACTGACCTGTAGGAGCTCCACAGGAGCTGTGACAACAAGCAATTACGCTAAC
TGGTTCCAGCAGAAGCCCGGTCAAGCCCCTAGAACCCTCATCGGCGGCACCAATAACAGA
GCTCCCGGAGTCCCCGCCAGGTTCTCCGGCTCCCTCCTGGGTGGCAAGGCTGCTCTGACA
CTCAGCGGTGCCCAGCCAGAGGATGAAGCGGAGTACTACTGTGCACTGTGGTACAGCAAC
CATTGGGTTTTCGGAGGCGGAACAAAGTTAACCGTCCTCGGGCAGCCTAAGGCTGCTCCT
AGCGTCACACTGTTCCCCCCATCTAGCGAGGAGCTGCAGGCTAACAAGGCAACCCTCGTC
TGCCTGGTTAGCGACTTCTACCCTGGCGCTGTCACAGTGGCCTGGAAAGCTGACGGCTCC
CCTGTGAAAGTTGGCGTCGAAACCACAAAGCCTTCTAAGCAGAGCAATAATAAATATGCC
GCAAGCTCCTACCTCTCCCTGACTCCTGAGCAGTGGAAAAGCCATAGGAGCTACTCCTGC
CGGGTCACACACGAAGGAAGCACAGTGGAAAAGACAGTCGCCCCTGCTGAGTGTAGCTGA
52M51-L3 Light chain amino acid sequence-predicted signal
sequence underlined
(SEQ ID NO: 28)
MSVPTMAWMMLLLGLLAYGSGVDSQAVVTQEPSLTVSPGGTVTLTCRSSTGAVTTSNYAN
WFQQKPGQAPRTLIGGTNNRAPGVPARFSGSLLGGKAALTLSGAQPEDEAEYYCALWYSN
HWVFGGGTKLTVLGQPKAAPSVTLFPPSSEELQANKATLVCLVSDFYPGAVTVAWKADGS
PVKVGVETTKPSKQSNNKYAASSYLSLTPEQWKSHRSYSCRVTHEGSTVEKTVAPAECS
52M51-L3 Light chain amino acid sequence without predicted
signal sequence
(SEQ ID NO: 29)
SGVDSQAVVTQEPSLTVSPGGTVTLTCRSSTGAVTTSNYANWFQQKPGQAPRTLIGGTNN
RAPGVPARFSGSLLGGKAALTLSGAQPEDEAEYYCALWYSNHWVFGGGTKLTVLGQPKAA
PSVTLFPPSSEELQANKATLVCLVSDFYPGAVTVAWKADGSPVKVGVETTKPSKQSNNKY
AASSYLSLTPEQWKSHRSYSCRVTHEGSTVEKTVAPAECS
52M51-L3 Light chain variable region polynucleotide sequence
(SEQ ID NO: 30)
ATGAGCGTCCCTACAATGGCTTGGATGATGCTCCTGCTGGGACTCCTGGCTTATGGAAGC
GGAGTGGATAGCCAGGCCGTCGTCACACAGGAACCTAGCCTCACCGTTAGCCCTGGAGGA
ACAGTCACACTGACCTGTAGGAGCTCCACAGGAGCTGTGACAACAAGCAATTACGCTAAC
TGGTTCCAGCAGAAGCCCGGTCAAGCCCCTAGAACCCTCATCGGCGGCACCAATAACAGA
GCTCCCGGAGTCCCCGCCAGGTTCTCCGGCTCCCTCCTGGGTGGCAAGGCTGCTCTGACA
CTCAGCGGTGCCCAGCCAGAGGATGAAGCGGAGTACTACTGTGCACTGTGGTACAGCAAC
CATTGGGTTTTCGGAGGCGGAACAAAGTTAACCGTCCTCGGG
52M51-L3 Light chain variable region amino acid sequence-
predicted signal sequence underlined
(SEQ ID NO: 31)
MSVPTMAWMMLLLGLLAYGSGVDSQAVVTQEPSLTVSPGGTVTLTCRSSTGAVTTSNYAN
WFQQKPGQAPRTLIGGTNNRAPGVPARFSGSLLGGKAALTLSGAQPEDEAEYYCALWYSN
HWVFGGGTKLTVLG
52M51-L3 Light chain variable region amino acid sequence without
predicted signal sequence
(SEQ ID NO: 32)
SGVDSQAVVTQEPSLTVSPGGTVTLTCRSSTGAVTTSNYANWFQQKPGQAPRTLIGGTNN
RAPGVPARFSGSLLGGKAALTLSGAQPEDEAEYYCALWYSNHWVFGGGTKLTVLG
52M51-L4 Light chain polynucleotide sequence
(SEQ ID NO: 33)
ATGAGCGTCCCTACAATGGCTTGGATGATGCTCCTGCTGGGACTCCTGGCTTATGGAAGC
GGAGTGGATAGCCAGACCGTCGTCACACAGGAACCTAGCTTTTCCGTTAGCCCTGGAGGA
ACAGTCACACTGACCTGTAGGAGCTCCACAGGAGCTGTGACAACAAGCAATTACGCTAAC
TGGTATCAGCAGACTCCCGGTCAAGCCCCTAGAACCCTCATCGGCGGCACCAATAACAGA
GCTCCCGGAGTCCCCGACAGGTTCTCCGGCTCCATCCTGGGAAATAAAGCTGCTCTGACA
ATCACAGGTGCCCAGGCTGACGATGAAAGCGACTACTACTGTGCACTGTGGTACAGCAAC
CATTGGGTTTTCGGAGGCGGAACAAAGTTAACCGTCCTCGGGCAGCCTAAGGCTGCTCCT
AGCGTCACACTGTTCCCCCCATCTAGCGAGGAGCTGCAGGCTAACAAGGCAACCCTCGTC
TGCCTGGTTAGCGACTTCTACCCTGGCGCTGTCACAGTGGCCTGGAAAGCTGACGGCTCC
CCTGTGAAAGTTGGCGTCGAAACCACAAAGCCTTCTAAGCAGAGCAATAATAAATATGCC
GCAAGCTCCTACCTCTCCCTGACTCCTGAGCAGTGGAAAAGCCATAGGAGCTACTCCTGC
CGGGTCACACACGAAGGAAGCACAGTGGAAAAGACAGTCGCCCCTGCTGAGTGTAGCTGA
52M51-L4 Light chain amino acid sequence-predicted signal
sequence underlined
(SEQ ID NO: 34)
MSVPTMAWMMLLLGLLAYGSGVDSQTVVTQEPSFSVSPGGTVTLTCRSSTGAVTTSNYAN
WYQQTPGQAPRTLIGGTNNRAPGVPDRFSGSILGNKAALTITGAQADDESDYYCALWYSN
HWVFGGGTKLTVLGQPKAAPSVTLFPPSSEELQANKATLVCLVSDFYPGAVTVAWKADGS
PVKVGVETTKPSKQSNNKYAASSYLSLTPEQWKSHRSYSCRVTHEGSTVEKTVAPAECS
52M51-L4 Light chain amino acid sequence without predicted
signal sequence
(SEQ ID NO: 35)
SGVDSQTVVTQEPSFSVSPGGTVTLTCRSSTGAVTTSNYANWYQQTPGQAPRTLIGGTNN
RAPGVPDRFSGSILGNKAALTITGAQADDESDYYCALWYSNHWVFGGGTKLTVLGQPKAA
PSVTLFPPSSEELQANKATLVCLVSDFYPGAVTVAWKADGSPVKVGVETTKPSKQSNNKY
AASSYLSLTPEQWKSHRSYSCRVTHEGSTVEKTVAPAECS
52M51-L4 Light chain variable region polynucleotide sequence
(SEQ ID NO: 36)
ATGAGCGTCCCTACAATGGCTTGGATGATGCTCCTGCTGGGACTCCTGGCTTATGGAAGC
GGAGTGGATAGCCAGACCGTCGTCACACAGGAACCTAGCTTTTCCGTTAGCCCTGGAGGA
ACAGTCACACTGACCTGTAGGAGCTCCACAGGAGCTGTGACAACAAGCAATTACGCTAAC
TGGTATCAGCAGACTCCCGGTCAAGCCCCTAGAACCCTCATCGGCGGCACCAATAACAGA
GCTCCCGGAGTCCCCGACAGGTTCTCCGGCTCCATCCTGGGAAATAAAGCTGCTCTGACA
ATCACAGGTGCCCAGGCTGACGATGAAAGCGACTACTACTGTGCACTGTGGTACAGCAAC
CATTGGGTTTTCGGAGGCGGAACAAAGTTAACCGTCCTCGGG
52M51-L4 Light chain variable region amino acid sequence-
predicted signal sequence is underlined
(SEQ ID NO: 37)
MSVPTMAWMMLLLGLLAYGSGVDSQTVVTQEPSFSVSPGGTVTLTCRSSTGAVTTSNYAN
WYQQTPGQAPRTLIGGTNNRAPGVPDRFSGSILGNKAALTITGAQADDESDYYCALWYSN
HWVFGGGTKLTVLG
52M51-L4 Light chain variable region amino acid sequence without
predicted signal sequence
(SEQ ID NO: 38)
SGVDSQTVVTQEPSFSVSPGGTVTLTCRSSTGAVTTSNYANWYQQTPGQAPRTLIGGTNN
RAPGVPDRFSGSILGNKAALTITGAQADDESDYYCALWYSNHWVFGGGTKLTVLG
FLAG-tag
(SEQ ID NO: 39)
DYKDDDDK
Human p53
(SEQ ID NO: 40)
MEEPQSDPSVEPPLSQETFSDLWKLLPENNVLSPLPSQAMDDLMLSPDDIEQWFTEDPGP
DEAPRMPEAAPPVAPAPAAPTPAAPAPAPSWPLSSSVPSQKTYQGSYGFRLGFLHSGTAK
SVTCTYSPALNKMFCQLAKTCPVQLWVDSTPPPGTRVRAMAIYKQSQHMTEVVRRCPHHE
RCSDSDGLAPPQHLIRVEGNLRVEYLDDRNTFRHSVVVPYEPPEVGSDCTTIHYNYMCNS
SCMGGMNRRPILTIITLEDSSGNLLGRNSFEVRVCACPGRDRRTEEENLRKKGEPHHELP
PGSTKRALPNNTSSSPQPKKKPLDGEYFTLQIRGRERFEMFRELNEALELKDAQAGKEPG
GSRAHSSHLKSKKGQSTSRHKKLMFKTEGPDSD
Human Notch1
(SEQ ID NO: 41)
MPPLLAPLLCLALLPALAARGPRCSQPGETCLNGGKCEAANGTEACVCGGAFVGPRCQDP
NPCLSTPCKNAGTCHVVDRRGVADYACSCALGFSGPLCLTPLDNACLTNPCRNGGTCDLL
TLTEYKCRCPPGWSGKSCQQADPCASNPCANGGQCLPFEASYICHCPPSFHGPTCRQDVN
ECGQKPGLCRHGGTCHNEVGSYRCVCRATHTGPNCERPYVPCSPSPCQNGGTCRPTGDVT
HECACLPGFTGQNCEENIDDCPGNNCKNGGACVDGVNTYNCRCPPEWTGQYCTEDVDECQ
LMPNACQNGGTCHNTHGGYNCVCVNGWTGEDCSENIDDCASAACFHGATCHDRVASFYCE
CPHGRTGLLCHLNDACISNPCNEGSNCDTNPVNGKAICTCPSGYTGPACSQDVDECSLGA
NPCEHAGKCINTLGSFECQCLQGYTGPRCEIDVNECVSNPCQNDATCLDQIGEFQCICMP
GYEGVHCEVNTDECASSPCLHNGRCLDKINEFQCECPTGFTGHLCQYDVDECASTPCKNG
AKCLDGPNTYTCVCTEGYTGTHCEVDIDECDPDPCHYGSCKDGVATFTCLCRPGYTGHHC
ETNINECSSQPCRHGGTCQDRDNAYLCFCLKGTTGPNCEINLDDCASSPCDSGTCLDKID
GYECACEPGYTGSMCNINIDECAGNPCHNGGTCEDGINGFTCRCPEGYHDPTCLSEVNEC
NSNPCVHGACRDSLNGYKCDCDPGWSGTNCDINNNECESNPCVNGGTCKDMTSGYVCTCR
EGFSGPNCQTNINECASNPCLNQGTCIDDVAGYKCNCLLPYTGATCEVVLAPCAPSPCRN
GGECRQSEDYESFSCVCPTGWQGQTCEVDINECVLSPCRHGASCQNTHGGYRCHCQAGYS
GRNCETDIDDCRPNPCHNGGSCTDGINTAFCDCLPGFRGTFCEEDINECASDPCRNGANC
TDCVDSYTCTCPAGFSGIHCENNTPDCTESSCFNGGTCVDGINSFTCLCPPGFTGSYCQH
DVNECDSQPCLHGGTCQDGCGSYRCTCPQGYTGPNCQNLVHWCDSSPCKNGGKCWQTHTQ
YRCECPSGWTGLYCDVPSVSCEVAAQRQGVDVARLCQHGGLCVDAGNTHHCRCQAGYTGS
YCEDLVDECSPSPCQNGATCTDYLGGYSCKCVAGYHGVNCSEEIDECLSHPCQNGGTCLD
LPNTYKCSCPRGTQGVHCEINVDDCNPPVDPVSRSPKCFNNGTCVDQVGGYSCTCPPGFV
GERCEGDVNECLSNPCDARGTQNCVQRVNDFHCECRAGHTGRRCESVINGCKGKPCKNGG
TCAVASNTARGFICKCPAGFEGATCENDARTCGSLRCLNGGTCISGPRSPTCLCLGPFTG
PECQFPASSPCLGGNPCYNQGTCEPTSESPFYRCLCPAKFNGLLCHILDYSFGGGAGRDI
PPPLIEEACELPECQEDAGNKVCSLQCNNHACGWDGGDCSLNFNDPWKNCTQSLQCWKYF
SDGHCDSQCNSAGCLFDGFDCQRAEGQCNPLYDQYCKDHFSDGHCDQGCNSAECEWDGLD
CAEHVPERLAAGTLVVVVLMPPEQLRNSSFHFLRELSRVLHTNVVFKRDAHGQQMIFPYY
GREEELRKHPIKRAAEGWAAPDALLGQVKASLLPGGSEGGRRRRELDPMDVRGSIVYLEI
DNRQCVQASSQCFQSATDVAAFLGALASLGSLNIPYKIEAVQSETVEPPPPAQLHFMYVA
AAAFVLLFFVGCGVLLSRKRRRQHGQLWFPEGFKVSEASKKKRREPLGEDSVGLKPLKNA
SDGALMDDNQNEWGDEDLETKKFRFEEPVVLPDLDDQTDHRQWTQQHLDAADLRMSAMAP
TPPQGEVDADCMDVNVRGPDGFTPLMIASCSGGGLETGNSEEEEDAPAVISDFIYQGASL
HNQTDRTGETALHLAARYSRSDAAKRLLEASADANIQDNMGRTPLHAAVSADAQGVFQIL
IRNRATDLDARMHDGTTPLILAARLAVEGMLEDLINSHADVNAVDDLGKSALHWAAAVNN
VDAAVVLLKNGANKDMQNNREETPLFLAAREGSYETAKVLLDHFANRDITDHMDRLPRDI
AQERMHHDIVRLLDEYNLVRSPQLHGAPLGGTPTLSPPLCSPNGYLGSLKPGVQGKKVRK
PSSKGLACGSKEAKDLKARRKKSQDGKGCLLDSSGMLSPVDSLESPHGYLSDVASPPLLP
SPFQQSPSVPLNHLPGMPDTHLGIGHLNVAAKPEMAALGGGGRLAFETGPPRLSHLPVAS
GTSTVLGSSSGGALNFTVGGSTSLNGQCEWLSRLQSGMVPNQYNPLRGSVAPGPLSTQAP
SLQHGMVGPLHSSLAASALSQMMSYQGLPSTRLATQPHLVQTQQVQPQNLQMQQQNLQPA
NIQQQQSLQPPPPPPQPHLGVSSAASGHLGRSFLSGEPSQADVQPLGPSSLAVHTILPQE
SPALPTSLPSSLVPPVTAAQFLTPPSQHSYSSPVDNTPSHQLQVPEHPFLTPSPESPDQW
SSSSPHSNVSDWSEGVSSPPTSMQSQIARIPEAFK

What is claimed is:

1. A method of treating an adenoid cystic carcinoma in a subject comprising administering to the subject a therapeutically effective amount of a Notch1-binding agent.

2. A method of decreasing pain in a subject with adenoid cystic carcinoma comprising administering to the subject a therapeutically effective amount of a Notch1-binding agent.

3. The method of claim 2, wherein the pain is bone pain.

4. A method of inhibiting growth of an adenoid cystic carcinoma comprising contacting the carcinoma with an effective amount of a Notch1-binding agent.

5. A method of decreasing the size of an adenoid cystic carcinoma comprising contacting the carcinoma with an effective amount of a Notch1-binding agent.

6. The method of any one of claims 1 to 5, wherein the adenoid cystic carcinoma has an increased level or elevated level of Notch1 intracellular domain (ICD) as compared to a predetermined level of Notch1 ICD.

7. The method of any one of claims 1 to 6, wherein the adenoid cystic carcinoma comprises a Notch1 mutation.

8. The method of claim 7, wherein the Notch1 mutation is a missense, nonsense, or frameshift mutation.

9. The method of claim 7 or claim 8, wherein the Notch1 mutation is in the HD domain.

10. The method of claim 7 or claim 8, wherein the Notch1 mutation is in the TAD domain.

11. The method of claim 7 or claim 8, wherein the Notch1 mutation is in the PEST domain.

12. The method of any one of claims 7 to 11, wherein the Notch1 mutation is an activating mutation.

13. The method of any one of claims 7 to 12, wherein the Notch1 mutation increases Notch1 signaling.

14. The method of any one of claims 7 to 12, wherein the Notch1 mutation increases the level of Notch1 ICD.

15. A method of treating an adenoid cystic carcinoma in a subject comprising:
(a) determining if the adenoid cystic carcinoma has an increased level or elevated level of Notch1 ICD as compared to a predetermined level of Notch1 ICD, and
(b) administering to the subject a therapeutically effective amount of a Notch1-binding agent.

16. A method of treating an adenoid cystic carcinoma in a subject comprising:
(a) determining if the adenoid cystic carcinoma comprises a Notch1 mutation, and
(b) administering to the subject a therapeutically effective amount of a Notch1-binding agent.

17. A method of selecting a subject with an adenoid cystic carcinoma for treatment with a Notch1-binding agent, comprising:
(a) determining the level of Notch1 ICD in the adenoid cystic carcinoma; and
(b) selecting the subject for treatment with the Notch1-binding agent if the adenoid cystic carcinoma has an increased level or an elevated level of Notch1 ICD as compared to a predetermined level of Notch1 ICD.

18. A method of selecting a subject with an adenoid cystic carcinoma for treatment with a Notch1-binding agent, comprising:
(a) obtaining a sample from the subject;
(b) determining the level of Notch1 ICD in the sample; and
(c) selecting the subject for treatment with the Notch1-binding agent if the sample has an increased level or an elevated level of Notch1 ICD as compared to a predetermined level of Notch1 ICD.

19. A method of selecting a subject with an adenoid cystic carcinoma for treatment with a Notch1-binding agent, comprising:
(a) determining if the adenoid cystic carcinoma has a Notch1 mutation; and
(b) selecting the subject for treatment with the Notch1-binding agent if the adenoid cystic carcinoma has a Notch1 mutation.

20. A method of selecting a subject with adenoid cystic carcinoma for treatment with a Notch1-binding agent, comprising:
(a) obtaining a sample from the subject;
(b) determining if the sample has a Notch1 mutation; and
(c) selecting the subject for treatment with the Notch1-binding agent if the sample has a Notch1 mutation.

21. The method of any one of claims 17 to 20, comprising administering a therapeutically effective amount of a Notch1-binding agent to the subject.

22. The method of any one of claims 1 to 21, wherein the Notch1-binding agent is an antibody that specifically binds human Notch1.

23. The method of claim 22, wherein the antibody specifically binds the extracellular domain of human Notch1.

24. The method of claim 22 or claim 23, wherein the antibody specifically binds a non-ligand binding membrane proximal region of the extracellular domain of human Notch1.

25. The method of claim 24, wherein the non-ligand binding membrane proximal region of the Notch1 receptor comprises SEQ ID NO:2.

26. The method of any one of claims 22 to 25, wherein the antibody comprises:
a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17), and a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20).

27. The method of any one of claims 22 to 25, wherein the antibody is a recombinant antibody, an IgG1 antibody, an IgG2 antibody, a monoclonal antibody, a chimeric antibody, a humanized antibody, a human antibody, a bispecific antibody, or an antibody fragment comprising an antigen-binding site.

28. The method of any one of claims 22 to 27, wherein the antibody comprises:
(a) a heavy chain variable region having at least 90% sequence identity to SEQ ID NO:8 or SEQ ID NO:26; and/or
(b) a light chain variable region having at least 90% sequence identity to SEQ ID NO:14, SEQ ID NO:32, or SEQ ID NO:38.

29. The method of any one of claims 22 to 27, wherein the antibody comprises:
(a) a heavy chain variable region of SEQ ID NO:8 and a light chain variable region of SEQ ID NO:14;
(b) a heavy chain variable region of SEQ ID NO:26 and a light chain variable region of SEQ ID NO:32; or
(c) a heavy chain variable region of SEQ ID NO:26 and a light chain variable region of SEQ ID NO:38.

30. The method of any one of claims 22 to 25, wherein the antibody comprises the same heavy chain variable region and light chain variable region as an antibody encoded by the plasmid on deposit as ATCC Patent Deposit Designation PTA-9549.

31. The method of any one of claims 28 to 30, wherein the antibody is an IgG1 antibody, an IgG2 antibody, a monoclonal antibody, a recombinant antibody, a chimeric antibody, or an antibody fragment comprising an antigen-binding site.

32. The method of any one of claims 22 to 26, wherein the antibody is an antibody encoded by the plasmid on deposit as ATCC Patent Deposit Designation PTA-9549.

33. The method of any one of claims 22 to 26, wherein the antibody is a humanized version of the antibody produced by the hybridoma on deposit as ATCC Patent Deposit Designation PTA-9405.

34. The method of any one of claims 22 to 26, wherein the antibody is OMP-52M51-H4L3.

35. The method of any one of claims 1 to 34, wherein the adenoid cystic carcinoma is recurrent.

36. The method of any one of claims 1 to 34, wherein the adenoid cystic carcinoma has metastasized from an original site.

37. The method of any one of claims 1 to 3 or 6 to 36, wherein the subject has an elevated level of lactate dehydrogenase (LDH) as compared to a predetermined level or a normal level.

38. The method of claim 4 or claim 5, wherein the adenoid cystic carcinoma has an elevated level of LDH as compared to a predetermined level or a normal level.

39. The method of any one of claims 1 to 38, which is monitored by measuring the level of LDH in a sample.

40. The method of any one of claims 1 to 3, 6 to 16, or 21 to 39, which further comprises administering to the subject at least one additional therapeutic agent.

41. The method of claim 40, wherein the additional therapeutic agent is a chemotherapeutic agent.

42. The method of claim 40, wherein the additional therapeutic agent is an additional therapeutic antibody.

43. The method of any one of claims 1 to 3, 6 to 16, or 21 to 42, wherein the subject receives radiation treatment.

44. The method of claim 43, wherein the subject receives radiation treatment prior to treatment with the Notch1-binding agent.

45. The method of any one of claims 16, 19, or 20 to 44, wherein the Notch1 mutation is determined by a PCR-based assay, microarray analysis, or nucleic acid sequencing.

46. The method of any one of claims 15, 17, 18, or 21 to 44, wherein the level of Notch1 ICD is determined by an immunohistochemistry assay.

47. The method of claims 18 or 20 to 34, wherein the sample is a fresh tumor sample, a frozen tumor sample, or a formalin-fixed paraffin-embedded sample.

48. The method of any one of claims 1 to 3, 6 to 16, or 21 to 44, wherein the subject is human.

49. A method of selecting a subject with an adenoid cystic carcinoma for treatment with an antibody that specifically binds human Notch1, comprising:
(a) determining the level of Notch1 ICD in the adenoid cystic carcinoma;
(b) selecting the subject for treatment with the antibody if the adenoid cystic carcinoma has an increased level or elevated level of Notch1 ICD as compared to a predetermined level of Notch1 ICD; and
(c) administering a therapeutically effective amount of the antibody to the subject;
wherein the antibody comprises: a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17), and a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20).

50. A method of treating an adenoid cystic carcinoma in a subject, comprising:
(a) determining if the adenoid cystic carcinoma has an increased level or elevated level of Notch1 ICD as compared to a predetermined level of Notch1 ICD; and
(b) administering a therapeutically effective amount of an antibody that specifically binds human Notch1 to the subject;
wherein the antibody comprises: a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17), and a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20).

51. A method of selecting a subject with an adenoid cystic carcinoma for treatment with an antibody that specifically binds human Notch1, comprising:
(a) determining if the adenoid cystic carcinoma has a Notch1 mutation;
(b) selecting the subject for treatment with the antibody if the adenoid cystic carcinoma has a Notch1 mutation; and
(c) administering a therapeutically effective amount of the antibody to the subject;
wherein the antibody comprises: a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17), and a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20).

52. A method of treating an adenoid cystic carcinoma in a subject, comprising:
(a) determining if the adenoid cystic carcinoma has a Notch1 mutation; and
(b) administering a therapeutically effective amount of an antibody that specifically binds human Notch1 to the subject;
wherein the antibody comprises a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17), and a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20).

53. A method of treating an adenoid cystic carcinoma in a subject, comprising administering a therapeutically effective amount of an antibody that specifically binds human Notch1 to the subject;
wherein the antibody comprises a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17), and a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20); and
wherein the subject has been selected based on the adenoid cystic carcinoma having an increased level or elevated level of Notch1 ICD as compared to a predetermined level of Notch1 ICD.

54. A method of treating an adenoid cystic carcinoma in a subject, comprising administering a therapeutically effective amount of an antibody that specifically binds human Notch1 to the subject;
wherein the antibody comprises a heavy chain CDR1 comprising RGYWIE (SEQ ID NO:15), a heavy chain CDR2 comprising QILPGTGRTNYNEKFKG (SEQ ID NO:16), and a heavy chain CDR3 comprising FDGNYGYYAMDY (SEQ ID NO:17), and a light chain CDR1 comprising RSSTGAVTTSNYAN (SEQ ID NO:18), a light chain CDR2 comprising GTNNRAP (SEQ ID NO:19), and a light chain CDR3 comprising ALWYSNHWVFGGGTKL (SEQ ID NO:20); and
wherein the subject has been selected based on the adenoid cystic carcinoma having a Notch1 mutation.

55. The method of any one of claims 49 to 52, comprising obtaining a sample from the subject.

56. The method of claim 55, wherein the sample is a fresh tumor sample, a frozen tumor sample, or a formalin-fixed paraffin-embedded sample.

57. The method of any one of claims 49 to 56, wherein the subject is human.

58. The method of any one of claims 49, 50, 53, or 55 to 57, wherein the level of Notch1 ICD is determined by an immunohistochemistry (IHC) assay.

59. The method of claim 58, wherein the IHC assay comprises antibody D3B8.

60. The method of any one of claims 51, 52, or 54 to 57, wherein the Notch1 mutation is determined by a PCR-based assay, microarray analysis, or nucleic acid sequencing.

61. The method of any one of claims 49 to 60, wherein the antibody is a recombinant antibody, an IgG1 antibody, an IgG2 antibody, a monoclonal antibody, a chimeric antibody, a humanized antibody, a human antibody, a bispecific antibody, or an antibody fragment comprising an antigen-binding site.

62. The method of any one of claims 49 to 60, wherein the antibody comprises:
(a) a heavy chain variable region having at least 90% sequence identity to SEQ ID NO:8 or SEQ ID NO:26; and/or
(b) a light chain variable region having at least 90% sequence identity to SEQ ID NO:14, SEQ ID NO:32, or SEQ ID NO:38.

63. The method of any one of claims 49 to 60, wherein the antibody comprises:
(a) a heavy chain variable region of SEQ ID NO:8 and a light chain variable region of SEQ ID NO:14;
(b) a heavy chain variable region of SEQ ID NO:26 and a light chain variable region of SEQ ID NO:32; or
(c) a heavy chain variable region of SEQ ID NO:26 and a light chain variable region of SEQ ID NO:38.

64. The method of any one of claims 49 to 60, wherein the antibody comprises the same heavy chain variable region and light chain variable region as an antibody encoded by the plasmid on deposit as ATCC Patent Deposit Designation PTA-9549.

65. The method of any one of claims 62 to 64, wherein the antibody is an IgG1 antibody, an IgG2 antibody, a monoclonal antibody, a recombinant antibody, a chimeric antibody, or an antibody fragment comprising an antigen-binding site.

66. The method of any one of claims 49 to 60, wherein the antibody is an antibody encoded by the plasmid on deposit as ATCC Patent Deposit Designation PTA-9549.

67. The method of any one of claims 49 to 60, wherein the antibody is a humanized version of the antibody produced by the hybridoma on deposit as ATCC Patent Deposit Designation PTA-9405.

68. The method of any one of claims 49 to 60, wherein the antibody is OMP-52M51.
